(19) United States
(12) Patent Application Publication
(10) Pub. No.: US 2006/0154917 A1
(43) Pub. Date: Jul. 13, 2006

(54) Substituted (heterocycloalkyl)methyl azole derivatives as c5a receptor modulators

(75) Inventors: Suoming Zhang, Madison, CT (US); He Zhao, Madison, CT (US); Yang Gao, Madison, CT (US); Andrew Thurkauf, Ridgefield, CT (US); George D Maynard, Clinton, CT (US); Bertrand L Chenard, Waterford, CT (US); Robert Ohliger, Deep River, CT (US); John M Peterson, Durham, CT (US)

Correspondence Address: EDWARDS & ANGELL, LLP, P.O. BOX 55874, BOSTON, MA, 02205, US

(73) Assignee: Neurogen Corporation, Branford, CT (US)

(21) Appl. No.: 10/563,401

(22) PCT Filed: Jun. 30, 2004
(86) PCT No.: PCT/US04/21191

§ 371 Date: Jan. 3, 2006

Related U.S. Application Data
(60) Provisional application No. 60/484,684, filed on Jul. 3, 2003.

(51) Int. Cl. A61K 31/55; A61K 31/496; A61K 31/454; C07D 417/02; C07D 413/02

(52) U.S. Cl. 514/217.1; 514/254.02; 514/326; 540/603; 544/368; 546/208

(57) ABSTRACT

(Heterocycloalkyl)methyl azole derivatives of Formula (I) are provided: Formula I wherein A is oxygen, sulfur, or NR; J and K and each L are independently oxygen, sulfur, NH, or CH₂; and the remaining variables are defined herein. Such compounds are modulators of C5a receptors, and preferably bind to C5a receptors with high affinity and exhibit neutral antagonist or inverse agonist activity at C5a receptors. Also provided herein are pharmaceutical compositions comprising such compounds, as well as methods for using such compounds in treating a variety of inflammatory and immune system disorders.

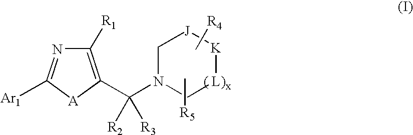

### FIELD OF THE INVENTION

This invention relates generally to substituted (heterocycloalkyl)methyl azole derivatives such as (heterocycloalkyl)methyl imidazole derivatives, (heterocycloalkyl)methyl oxazole derivatives, and (heterocycloalkyl)methyl thiazole derivatives, and to pharmaceutical compositions comprising such compounds. The present invention further relates to the use of such modulators in treating a variety of inflammatory and immune system disorders and as probes for the localization of C5a receptors.

### BACKGROUND OF THE INVENTION

C5a, a 74 amino acid peptide, is generated in the complement cascade by the cleavage of the complement protein C5 by the complement C5 convertase enzyme. C5a has both anaphylatoxic (e.g., bronchoconstricting and vascular spasmogenic) and chemotactic effects. Therefore, it is active in engendering both the vascular and cellular phases of inflammatory responses. Because it is a plasma protein and, therefore, generally almost instantly available at a site of an inciting stimulus, it is a key mediator in terms of initiating the complex series of events that results in augmentation and amplification of an initial inflammatory stimulus. The anaphylatoxic and chemotactic effects of the C5a peptide are believed to be mediated through its interaction with the C5a receptor (CD88 antigen), a 52 kD membrane bound G-protein coupled receptor (GPCR). C5a is a potent chemoattractant for polymorphonuclear leukocytes, bringing neutrophils, basophils, eosinophils and monocytes to sites of inflammation and/or cellular injury. C5a is one of the most potent chemotactic agents known for a wide variety of inflammatory cell types. C5a also “primes” or prepares neutrophils for various antibacterial functions (e.g., phagocytosis). Additionally, C5a stimulates the release of inflammatory mediators (e.g., histamines, TNF-α, IL-1, IL-6, IL-8, prostaglandins, and leukotrienes) and the release of lysosomal enzymes and other cytotoxic components from granulocytes. Among its other actions, C5a also promotes the production of activated oxygen radicals and the contraction of smooth muscle.

Considerable experimental evidence implicates increased levels of C5a in a number of autoimmune diseases and inflammatory and related disorders. Agents that block the binding of C5a to its receptor other agents, including inverse agonists, which modulate signal transduction associated with C5a-receptor interactions, can inhibit the pathogenic events, including chemotaxis, associated with anaphylatoxin activity contributing to such inflammatory and autoimmune conditions. The present invention provides such agents, and has further related advantages.

### SUMMARY OF THE INVENTION

The present invention provides (heterocycloalkyl)methyl-azole compounds of Formula I, as well as pharmaceutically acceptable salts of such compounds.

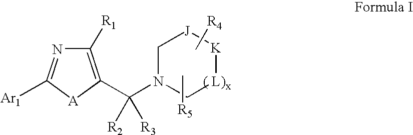

Within Formula I:
A is oxygen, sulfur or NR;
R is C₁-C₇alkyl, C₂-C₇alkenyl, C₂-C₇alkynyl, C₁-C₆haloalkyl, (C₃-C₁₀carbocycle)C₁-C₄alkyl or (4- to 7-membered heterocycloalkyl)C₁-C₄alkyl, each of which is optionally substituted, and preferably each of which is substituted with from 0 to 3 substituents independently chosen from hydroxy, halogen, amino, cyano, oxo, C₁-C₄alkyl, C₁-C₄alkoxy and C₁-C₂alkoxycarbonyl;
x is 0, 1 or 2;
J, K and each occurrence of L are chosen from oxygen, sulfur, NH and CH₂; such that no more than one of J, K and L is chosen from oxygen, sulfur and NH;
R₁ is chosen from:
   i) hydrogen, hydroxy, halogen, amino, cyano, nitro, —CHO, —CONH₂, C₁-C₆haloalkyl and C₁-C₆haloalkoxy;
   ii) C₁-C₆alkyl, C₂-C₇alkenyl, C₂-C₇alkynyl, C₂-C₆alkanoyl, C₁-C₆alkoxy, (C₃-C₇cycloalkyl)C₀-C₄alkyl, (4- to 10-membered heterocycloalkyl)C₀-C₄alkyl, mono- and di-(C₁-C₆alkyl)aminoC₀-C₆alkyl, mono- and di-(C₁-C₆alkyl)carboxamide, C₁-C₆alkoxycarbonyl, —SOₙ(C₁-C₆alkyl), —NHSOₙC₁-C₆alkyl, —(C₀-C₆alkyl)SOₙ(C₁-C₆alkyl), —SOₙN(C₁-C₆alkyl)(C₁-C₆alkyl), and —SOₙ-phenyl (wherein each n is independently 0, 1 or 2), each of which is optionally substituted, and preferably each of which is optionally substituted with from 0 to 3 substituents independently chosen from hydroxy, halogen, amino, cyano, oxo, C₁-C₄alkyl, C₁-C₄alkoxy and C₁-C₂alkoxycarbonyl; and
   iii) naphthyl, phenyl and 5- to 10-membered heteroaryl, each of which is optionally substituted and is preferably substituted with from 0 to 3 substituents independently chosen from R₁₁;
R₂ and R₃ are independently hydrogen or C₁-C₆alkyl;
R₄ represents 1 substituent chosen from:
   i) C₁-C₆alkyl, C₂-C₆alkenyl, C₂-C₆alkynyl, C₁-C₆alkoxycarbonyl, (C₃-C₇cycloalkyl)C₀-C₄alkyl and hexahydro-1,3-benzodioxolyl, each of which is optionally substituted;
   ii) optionally substituted aryl having 1 ring or 2 fused or pendant rings;
   iii) optionally substituted (4- to 10-membered heterocycloalkyl)C₀-C₄alkyl;
   iv) optionally substituted phenyl fused to a 5- to 7-membered saturated or partially unsaturated ring that (a) has 0, 1 or 2 ring atoms independently chosen from N, O and S, with remaining ring atoms being carbon, and (b) is substituted with from 0 to 3 substituents independently chosen from halogen, C₁-C₈alkyl, C₁-C₈alkoxy, C₁-C₈haloalkyl, C₁-C₈haloalkoxy;
   v) optionally substituted (5- to 10-membered heteroaryl)C₀-C₄alkyl, having 1 ring or 2 fused or pendant rings, from 5 to 7 members in each ring, and in at least one ring from 1 to 3 heteroatoms independently selected from N, O, and S, wherein R₄ is not pyrimidyl; and
   vi) groups that are taken together with an R₅ moiety to form a fused phenyl or pyridyl ring, each of which is optionally substituted;
   wherein each of i), ii), iii), iv), v) and vi) is preferably substituted with from 0 to 3 substituents independently chosen from R₁₁;
R₅ represents from 0 to 3 substituents independently chosen from hydroxy, halogen, amino, cyano, nitro, —CHO, —CONH₂, C₁-C₂haloalkyl, C₁-C₂haloalkoxy, C₁-C₆alkyl, C₁-C₆alkoxy, C₃-C₇cycloalkylC₀-C₄alkyl, mono- and di-(C₁-C₆alkyl)aminoC₀-C₆alkyl, optionally substituted phenyl, and groups that are taken together with R₄ to form a fused, optionally substituted phenyl or pyridyl ring;
Ar₁ represents
   i) optionally substituted aryl, preferably phenyl or naphthyl, each of which is substituted with from 0 to 3 substituents independently chosen from amino, cyano, C₁-C₄alkyl, C₁-C₄alkoxy, C₁-C₂haloalkyl, C₁-C₂haloalkoxy, —COOH, —CONH₂, mono- and di-(C₁-C₄alkyl)amino, C₂-C₄alkanoyl, C₁-C₄sulfonate, C₁-C₄alkylsulfonyl, C₁-C₄alkylsulfinyl, C₁-C₄alkylthio, C₃-C₆alkanone, C₂-C₄alkyl ether, C₂-C₄alkanoyloxy, C₁-C₄alkoxycarbonyl and C₁-C₆alkylcarboxamide;
   ii) optionally substituted phenyl fused to a 5- to 7-membered saturated or partially unsaturated ring that (a) has 0, 1 or 2 ring atoms independently chosen from N, O and S, with remaining ring atoms being carbon, and (b) is substituted with from 0 to 3 substituents independently chosen from halogen, C₁-C₄alkyl, C₁-C₄alkoxy, C₁-C₂haloalkyl and C₁-C₂haloalkoxy; or
   iii) optionally substituted heteroaryl, having 1 ring or 2 fused or pendant rings, from 5 to 7 members in each ring, and in at least one ring from 1 to 3 heteroatoms independently selected from N, O, and S;
   wherein each of ii) and iii) is preferably substituted with from 0 to 3 substituents independently chosen from R₁₁; and
R₁₁ is independently chosen at each occurrence from hydroxy, halogen, amino, cyano, C₁-C₆alkyl, C₁-C₆alkoxy, C₁-C₆haloalkyl, C₁-C₆haloalkoxy, —COOH, —CONH₂, —SO₂NH₂, mono- and di-(C₁-C₆alkyl)amino, C₂-C₆alkanoyl, C₁-C₆sulfonate, C₁-C₆alkylsulfonyl, C₁-C₆alkylsulfinyl, C₁-C₆alkylthio, C₃-C₆alkanone, C₂-C₆alkyl ether, C₂-C₆alkanoyloxy, C₁-C₆alkoxycarbonyl and C₁-C₆alkylcarboxamide.

In certain aspects, such compounds are C5a receptor modulators that alter, and preferably inhibit, C5a receptor activation and/or C5a receptor-mediated signal transduction. Such C5a receptor modulators are preferably high affinity C5a receptor ligands and act as antagonists (e.g., inverse agonists) of complement C5a receptors, such as human C5a receptors. Within certain aspects, compounds as described herein exhibit an IC₅₀ of 500 nM or less, or 25 nM or less, in a standard in vitro C5a receptor-mediated chemotaxis or calcium mobilization assay.

Within further aspects, compounds as described herein exhibit less than 5% agonist activity in a GTP binding assay.

The present invention further provides, within other aspects, pharmaceutical compositions comprising at least one compound as described herein, in combination with a physiologically acceptable carrier or excipient.

Within further aspects, methods are provided for inhibiting signal-transducing activity of a cellular C5a receptor, comprising contacting a cell expressing a C5a receptor with at least one compound as described herein, and thereby reducing signal transduction by the C5a receptor.

Methods are further provided for inhibiting binding of C5a to C5a receptor in vitro, the method comprising contacting C5a receptor with at least one compound as described herein, under conditions and in an amount sufficient to detectably inhibit C5a binding to C5a receptor.

The present invention further provides methods for inhibiting binding of C5a to C5a receptor in a human patient, comprising contacting cells expressing C5a receptor with at least one compound as described herein, in an amount sufficient to detectably inhibit C5a binding to cells expressing a cloned C5a receptor in vitro.

Within other aspects, methods are provided for treating a patient suffering from rheumatoid arthritis, psoriasis, cardiovascular disease, reperfusion injury, or bronchial asthma comprising administering to the patient a C5a receptor modulatory amount of a compound as described herein.

Methods are further provided for treating a patient suffering from stroke, myocardial infarction, atherosclerosis, ischemic heart disease, or ischemia-reperfusion injury comprising administering to the patient a C5a receptor modulatory amount of a compound as described herein.

The present invention further provides methods for inhibiting C5a receptor-mediated cellular chemotaxis, comprising contacting mammalian white blood cells with a C5a receptor modulatory amount of a compound as described herein.

Within other aspects, the present invention provides methods for localizing C5a receptors in a tissue sample, comprising: (a) contacting the tissue sample containing C5a receptors with a detectably labeled compound as described herein under conditions that permit binding of the compound to C5a receptors; and (b) detecting the bound compound.

The present invention also provides packaged pharmaceutical preparations, comprising: (a) a pharmaceutical composition as described herein in a container; and (b) instructions for using the composition to treat a patient suffering from one or more conditions responsive to C5a receptor modulation, such as rheumatoid arthritis, psoriasis, cardiovascular disease, reperfusion injury, bronchial asthma, stroke, myocardial infarction, atherosclerosis, ischemic heart disease, or ischemia-reperfusion injury.

In yet another aspects, the present invention provides methods for preparing the compounds disclosed herein, including the intermediates.

These and other aspects of the present invention will become apparent upon reference to the following detailed description

### DETAILED DESCRIPTION OF THE INVENTION

As noted above, the present invention provides substituted (heterocycloalkyl)methyl azole derivatives of Formula I, and more particularly provides (heterocycloalkyl)methylimidazole, (heterocycloalkyl)methyloxazole, and (heterocycloalkyl)methylthiazole derivatives of Formula I. In certain aspects, such compounds modulate C5a receptor activation and/or C5a receptor-mediated signal transduction. Such compounds may be used in vitro or in vivo to modulate (preferably inhibit) C5a receptor activity in a variety of contexts.

Chemical Description and Terminology

Compounds provided herein are generally described using standard nomenclature. For compounds having asymmetric centers, it should be understood that (unless otherwise specified) all of the optical isomers and mixtures thereof are encompassed. Compounds with two or more asymmetric elements can also be present as mixtures of diastereomers. In addition, compounds with carbon-carbon double bonds may occur in Z- and E-forms, with all isomeric forms of the compounds being included in the present invention unless otherwise specified. Where a compound exists in various tautomeric forms, a recited compound is not limited to any one specific tautomer, but rather is intended to encompass all tautomeric forms. Recited compounds are further intended to encompass compounds in which one or more atoms are replaced with an isotope (i.e., an atom having the same atomic number but a different mass number). By way of general example, and without limitation, isotopes of hydrogen include tritium and deuterium and isotopes of carbon include ¹¹C, ¹³C and ¹⁴C.

Certain compounds are described herein using a general formula that includes variables (e.g., R, R₁-R₆, Ar₁). Unless otherwise specified, each variable within such a formula is defined independently of any other variable, and any variable that occurs more than one time in a formula is defined independently at each occurrence. Thus, for example, if a group is shown to be substituted with 0-2R*, the group may be unsubstituted or substituted with up to two R* groups and R* at each occurrence is selected independently from the definition of R*. Also, combinations of substituents and/or variables are permissible only if such combinations result in stable compounds.

The term “(heterocycloalkyl)methylazole derivatives” as used herein, encompasses all compounds that satisfy Formula I, as well as pharmaceutically acceptable salts thereof. In other words, this term encompasses compounds in which A is N, as well as related compounds in which A is O or S. Such compounds may, but need not, further satisfy one or more additional Formulas provided herein.

A “pharmaceutically acceptable salt” of a compound recited herein is an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is 0-4, and the like. Similarly, pharmaceutically acceptable cations include, but are not limited to sodium, potassium, calcium, aluminum, lithium and ammonium. Those of ordinary skill in the art will recognize further pharmaceutically acceptable salts for the compounds provided herein, including those listed by Remington's Pharmaceutical Sciences, 17th ed., Mack Publishing Company, Easton, Pa., p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent, or in a mixture of the two; generally, the use of nonaqueous media, such as ether, ethyl acetate, ethanol, isopropanol or acetonitrile, is preferred.

It will be apparent that each compound of Formula I may, but need not, be formulated as a hydrate, solvate or non-covalent complex. In addition, the various crystal forms and polymorphs are within the scope of the present invention. Also provided herein are prodrugs of the compounds of Formula I. A “prodrug” is a compound that may not fully satisfy the structural requirements of the compounds provided herein, but is modified in vivo, following administration to a patient, to produce a compound of Formula I, or other formula provided herein. For example, a prodrug may be an acylated derivative of a compound as provided herein. Prodrugs include compounds wherein hydroxy, amine or sulfhydryl groups are bonded to any group that, when administered to a mammalian subject, cleaves to form a free hydroxy, amino, or sulfhydryl group, respectively. Examples of prodrugs include, but are not limited to, acetate, formate and benzoate derivatives of alcohol and amine functional groups within the compounds provided herein. Prodrugs of the compounds provided herein may be prepared by modifying functional groups present in the compounds in such a way that the modifications are cleaved to the parent compounds.

A “C5a receptor modulatory amount” is an amount that, upon administration, results in a concentration of C5a receptor modulator at a C5a receptor that is sufficient to inhibit chemotaxis of white blood cells in an in vitro assay and/or alter C5a receptor activity or activation as measured by an in vitro calcium mobilization assay. In a chemotaxis assay (see Example 10), the level of C5a-induced chemotaxis observed in a control assay (i.e., one to which a compound as provided herein has not been added) is significantly higher (measured as p≦0.05 using a conventional parametric statistical analysis method such as a student's T-test) than the level observed in an assay to which a compound as described herein has been added. Within such an assay, the C5a is generally from the same species as the cells used in the assay. In a calcium mobilization assay (see Example 17), a concentration of compound that alters C5a receptor activity or activation may inhibit C5a-induced calcium mobilization or may itself increase or decrease C5a receptor-mediated calcium mobilization in the absence of C5a.

A “therapeutically effective amount” is an amount of a compound as provided herein that, upon administration, results in a discernible benefit in a patient. Such benefit may be confirmed using standard clinical procedures.

A “substituent,” as used herein, refers to a molecular moiety that is covalently bonded to an atom within a molecule of interest. For example, a “ring substituent” may be a moiety such as a halogen, alkyl group, haloalkyl group or other substituent described herein that is covalently bonded to an atom (preferably a carbon or nitrogen atom) that is a ring member. The term “substituted,” as used herein, means that any one or more hydrogens on the designated atom is replaced with a selection from the indicated substituents, provided that the designated atom's normal valence is not exceeded, and that the substitution results in a stable compound (i.e., a compound that can be isolated, characterized and tested for biological activity). When a substituent is oxo (i.e., =0), then 2 hydrogens on the atom are replaced. When aromatic moieties are substituted by an oxo group, the aromatic ring is replaced by the corresponding partially unsaturated ring. For example a pyridyl group substituted by oxo is a pyridone.

The phrase “optionally substituted” indicates that a group may either be unsubstituted or substituted at one or more of any of the available positions, typically 1, 2, 3, 4, or 5 positions, by one or more suitable substituents such as those disclosed herein. Optional substitution may also be indicated by the phrase “substituted with from 0 to X substituents,” in which X is the maximum number of substituents.

Suitable substituents include, for example, halogen, cyano, amino, hydroxy, nitro; azido, carboxamido, —COOH, SO₂NH₂, alkyl (e.g., C₁-C₈alkyl), alkenyl (e.g., C₂-C₈alkenyl), alkynyl (e.g., C₂-C₈alkynyl), alkoxy (e.g., C₁-C₈alkoxy), alkyl ether (e.g., C₂-C₈alkyl ether), alkylthio (e.g., C₁-C₈alkylthio), haloalkyl (e.g., C₁-C₈haloalkyl), hydroxyalkyl (e.g., C₁-C₈hydroxyalkyl), aminoalkyl (e.g., C₁-C₈aminoalkyl), haloalkoxy (e.g., C₁-C₈haloalkoxy), alkanoyl (e.g., C₂-C₈alkanoyl), alkanone (e.g., C₃-C₈alkanone), alkanoyloxy (e.g., C₂-C₈alkanoyloxy), alkoxycarbonyl (e.g., C₁-C₈alkoxycarbonyl), mono- and di-(C₁-C₈alkyl)amino, mono- and di-(C₁-C₈alkyl)aminoC₁-C₈alkyl, mono- and di-(C₁-C₈alkyl)carboxamido, mono- and di-(C₁-C₈alkyl)sulfonamido, alkylsulfinyl (e.g., C₁-C₈alkylsulfinyl), alkylsulfonyl (e.g., C₁-C₈alkylsulfonyl), aryl (e.g., phenyl), arylalkyl (e.g., (C₆-C₁₈aryl)C₁-C₈alkyl, such as benzyl and phenethyl), aryloxy (e.g., C₆-C₁₈aryloxy such as phenoxy), arylalkoxy (e.g., (C₆-C₁₈aryl)C₁-C₈alkoxy) and/or 3- to 8-membered heterocyclic groups such as coumarinyl, quinolinyl, pyridyl, pyrazinyl, pyrimidyl, furyl, pyrrolyl, thienyl, thiazolyl, oxazolyl, imidazolyl, indolyl, benzofuranyl, benzothiazolyl, tetrahydrofuranyl, tetrahydropyranyl, piperidinyl, morpholino or pyrrolidinyl. Certain groups within the formulas provided herein are optionally substituted with from 1 to 3, 1 to 4 or 1 to 5 independently selected substituents.

A dash (“-”) that is not between two letters or symbols is used to indicate a point of attachment for a substituent. For example, —CONH₂ is attached through the carbon atom.

As used herein, “alkyl” is intended to include both branched and straight-chain saturated aliphatic hydrocarbon groups. Alkyl groups include groups having from 1 to 8 carbon atoms (C₁-C₈alkyl), from 1 to 6 carbon atoms (C₁-C₆alkyl) and from 1 to 4 carbon atoms (C₁-C₄alkyl), such as methyl, ethyl, n-propyl, isopropyl, n-butyl, sec-butyl, tert-butyl, pentyl, 2-pentyl, isopentyl, neopentyl, hexyl, 2-hexyl, 3-hexyl, and 3-methylpentyl. In certain embodiments, preferred alkyl groups are methyl, ethyl, propyl, butyl, and 3-pentyl. “C₀-C₄alkyl” refers to a single covalent bond or a C₁-C₄alkyl group. “Aminoalkyl” is an alkyl group as defined herein substituted with one or more —NH₂ substituents. “Hydroxyalkyl” is a hydroxy group as defined herein substituted with one or more —OH substituents.

“Alkenyl” refers to a straight or branched hydrocarbon chain comprising one or more unsaturated carbon-carbon bonds, such as ethenyl and propenyl. Alkenyl groups include C₂-C₈alkenyl, C₂-C₆alkenyl and C₂-C₄alkenyl groups (which have from 2 to 8, 2 to 6 or 2 to 4 carbon atoms, respectively), such as ethenyl, allyl or isopropenyl.

“Alkynyl” refers to straight or branched hydrocarbon chains comprising one or more triple carbon-carbon bonds. Alkynyl groups include C₂-C₈alkynyl, C₂-C₆alkynyl and C₂-C₄alkynyl groups, which have from 2 to 8, 2 to 6 or 2 to 4 carbon atoms, respectively. Alkynyl groups include for example groups such as ethynyl and propynyl.

By “alkoxy,” as used herein, is meant an alkyl, alkenyl or alkynyl group as described above attached via an oxygen bridge. Alkoxy groups include C₁-C₆alkoxy and C₁-C₄alkoxy groups, which have from 1 to 6 or 1 to 4 carbon atoms, respectively. Methoxy, ethoxy, propoxy, isopropoxy, n-butoxy, sec-butoxy, tert-butoxy, n-pentoxy, 2-pentoxy, 3-pentoxy, isopentoxy, neopentoxy, hexoxy, 2-hexoxy, 3-hexoxy, and 3-methylpentoxy are specific alkoxy groups. Similarly “alkylthio” refers to an alkyl, alkenyl or alkynyl group as described above attached via a sulfur bridge.

The term “alkanoyl” refers to an alkyl group as defined above attached through a carbonyl bridge. Alkanoyl groups include C₂-C₈alkanoyl, C₂-C₆alkanoyl and C₂-C₄alkanoyl groups, which have from 2 to 8, 2 to 6 or 2 to 4 carbon atoms, respectively.

An “alkanone” is an alkyl group as defined above with the indicated number of carbon atoms substituted at least one position with an oxo group. “C₃-C₈alkanone,” “C₃-C₆alkanone” and “C₃-C₄alkanone” refer to an alkanone having from 3 to 8, 6 or 4 carbon atoms, respectively. By way of example, a C₃ alkanone group has the structure —CH₂—(C═O)—CH₃.

Similarly, “alkyl ether” refers to a linear or branched ether substituent linked via a carbon-carbon bond. Alkyl ether groups include C₂-C₈alkyl ether, C₂-C₆alkyl ether and C₂-C₄alkyl ether groups, which have 2 to 8, 6 or 4 carbon atoms, respectively. By way of example, a C₂ alkyl ether group has the structure —CH₂—O—CH₃.

The term “alkoxycarbonyl” refers to an alkoxy group linked via a carbonyl (i.e., a group having the general structure —C(═O)—O-alkyl). Alkoxycarbonyl groups include C₁-C₆, C₁-C₄ and C₁-C₂alkoxycarbonyl groups, in which the alkyl portion has from 1 to 6, 4 or 2 carbon atoms, respectively.

“Alkanoyloxy,” as used herein, refers to an alkanoyl group linked via an oxygen bridge (e.g., a group having the general structure —O—C(═O)-alkyl). Alkanoyloxy groups include C₂-C₈, C₂-C₆ and C₂-C₄alkanoyloxy groups, which have from 2 to 8, 6 or 4 carbon atoms, respectively.

“Alkylamino” refers to a secondary or tertiary amine having the general structure —NH-allyl or —N(alkyl)(alkyl), wherein each alkyl may be the same or different. Such groups include, for example, mono- and di-(C₁-C₈alkyl)amino groups, in which each alkyl may be the same or different and may contain from 1 to 8 carbon atoms, as well as mono- and di-(C₁-C₆alkyl)amino groups and mono- and di-(C₁-C₄alkyl)amino groups.

“Alkylaminoalkyl” refers to an alkylamino group linked via an alkyl group (i.e., a group having the general structure -alkyl-NH-alkyl or -alkyl-N(alkyl)(alkyl)) in which each alkyl is selected independently. Such groups include, for example, mono- and di-(C₁-C₈alkyl)aminoC₁-C₈alkyl, mono- and di-(C₁-C₆alkyl)aminoC₁-C₆alkyl and mono- and di-(C₁-C₄alkyl)aminoC₁-C₄alkyl, in which each alkyl may be the same or different. “Mono- or di-(C₁-C₆alkyl)aminoC₀-C₆alkyl” refers to a mono- or di-(C₁-C₆alkyl)amino group linked via a single covalent bond or a C₁-C₆alkyl group. The following are representative alkylaminoalkyl groups:

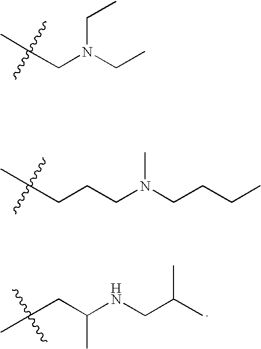

“Alkylsulfinyl,” as used herein, refers to an alkyl group attached via a sulfinyl linkage. Alkylsulfinyl groups include C₁-C₈alkylsulfinyl, C₁-C₆alkylsulfinyl, and C₁-C₄alkylsulfinyl, which have from 1 to 8, 1 to 6, and 1 to 4 carbon atoms, respectively.

By “alkylsulfonyl,” as used herein, is meant an alkyl group attached via a sulfonyl linkage. Alkylsulfonyl groups include C₁-C₈alkylsulfonyl, C₁-C₆alkylsulfonyl, and C₁-C₄alkylsulfonyl, which have from 1 to 8, 1 to 6, and 1 to 4 carbon atoms, respectively.

The term “alkylsulfonate” is used herein to refer to an alkyl group attached via a sulfonate linkage. Such groups include, for example —SO₂—O—(C₁-C₄alkyl).

The term “oxo,” as used herein, refers to a keto (C═O) group. An oxo group that is a substituent of a nonaromatic carbon atom results in a conversion of —CH₂— to —C(═O)—.

The term “aminocarbonyl” or “carboxamide” refers to an amide group (i.e., —(C═O)NH₂). “Mono- or di-(C₁-C₆alkyl)carboxamide” refers to an amide group in which one or both of the hydrogen atoms is replaced with an independently chosen C₁-C₆alkyl. Such groups may also be indicated by “—C(═O)NHalkyl” or “—C(═O)N(alkyl)(alkyl).”

The term “halogen” indicates fluorine, chlorine, bromine, or iodine.

A “haloalkyl” is a branched or straight-chain alkyl group, substituted with 1 or more halogen atoms (e.g., “haloC₁-C₈alkyl” groups have from 1 to 8 carbon atoms; “haloC₁-C₆alkyl” groups have from 1 to 6 carbon atoms). Examples of haloalkyl groups include, but are not limited to, mono-, di- or tri-fluoromethyl; mono-, di- or tri-chloromethyl; mono-, di-, tri-, tetra- or penta-fluoroethyl; and mono-, di-, tri-, tetra- or penta-chloroethyl. Typical haloalkyl groups are trifluoromethyl and difluoromethyl. Within certain compounds provided herein, not more than 5 or 3 haloalkyl groups are present. The term “haloalkoxy” refers to a haloalkyl group as defined above attached via an oxygen bridge. “HaloC₁-C₆alkoxy” groups have 1 to 6 carbon atoms.

A “carbocycle” is any saturated, partially saturated, or aromatic group having 1 or 2 fused, pendant or spiro rings, with 3 to 8 atoms in each ring, and with all ring members being carbon. The term “carbocycle” encompasses aromatic groups such as phenyl and naphthyl, as well as groups that comprise both aromatic and nonaromatic rings (e.g., tetrahydronaphthyl), and groups with saturated and partially saturated rings (such as cyclohexyl and cyclohexenyl). When substitutions are indicated, carbocycles may be substituted on any ring atom where such substitution results in a stable compound. The term “C₃-C₁₀carbocycle” refers to such groups having from 3 to 10 ring members. A “(C₃-C₁₀carbocycle)C₁-C₄alkyl” group is a C₃-C₁₀carbocycle that is linked via a C₁-C₄alkyl group. A “(C₃-C₁₀carbocycle)C₀-C₄alkyl” group is a C₃-C₁₀carbocycle that is linked via a single covalent bond or a C₁-C₄alkyl group.

Certain carbocycles are “cycloalkyl” (i.e., a saturated or partially saturated carbocycle). Such groups typically contain from 3 to about 8 ring carbon atoms; in certain embodiments, such groups have from 3 to 7 ring carbon atoms. Examples of cycloalkyl groups include cyclopropyl, cyclobutyl, cyclopentyl and cyclohexyl, as well as such groups modified by the presence of one or more double or triple bonds (e.g., cyclohexenyl) and bridged or caged saturated ring groups such as norbornane or adamantane. If substituted, any ring carbon atom may be bonded to any indicated substituent.

In the term “(cycloalkyl)alkyl”, “cycloalkyl” and “alkyl” are as defined above, and the point of attachment is on the alkyl group. This term encompasses, but is not limited to, cyclopropylmethyl, cyclohexylmethyl and cyclohexylethyl. “(C₃-C₇cycloalkyl)C₀-C₄alkyl” refers to 3- to 7-membered cycloalkyl rings that are linked via a direct bond or a C₁-C₄alkyl.

Other carbocycles are “aryl” (i.e., carbocycles that comprise at least one aromatic ring). In addition to the aromatic ring(s), additional non-aromatic ring(s) may be present in an aryl group. Representative aryl groups include phenyl, naphthyl (e.g., 1-naphthyl and 2-naphthyl), biphenyl, tetrahydronaphthyl and indanyl.

The term “arylalkyl” refers to an aryl group that is linked via an alkyl group. Certain arylalkyl groups are arylC₀-C₂alkyl, in which an aryl group is linked via a direct bond or a methylene or ethylene moiety. Such groups include, for example, groups in which phenyl or naphthyl is linked via a bond or C₁-C₂alkyl, such as benzyl, 1-phenyl-ethyl and 2-phenyl-ethyl.

The term “aryloxy” refers to an aryl group linked via a an oxygen (i.e., a group having the general structure —O-aryl). Phenoxy is a representative aryloxy group.

The term “azole” refers to a five membered heteroaryl group having a nitrogen ring atom and between 0 and 2 additional ring heteroatoms selected from N, O or S. Imidazole, oxazole, and thiazole are representative azole groups.

A “heteroatom” is an atom other than carbon, such as oxygen, sulfur or nitrogen.

The term “heterocycle” or “heterocyclic group” is used to indicate saturated, partially unsaturated, or aromatic groups having 1 or 2 fused, pendent or spiro rings, with 3 to 8 atoms in each ring, and in at least one ring from 1 to 4 heteroatoms independently selected from N, O and S, with remaining atoms being carbon. Certain heterocycles are 3- to 10-membered monocyclic or bicyclic groups; others are 4- to 6-membered monocyclic groups. The heterocyclic ring may be attached at any heteroatom or carbon atom that results in a stable structure, and may be substituted on carbon and/or nitrogen atom(s) if the resulting compound is stable. Any nitrogen and/or sulfur heteroatoms may optionally be oxidized, and any nitrogen may optionally be quaternized.

Variations on the term “(heterocycle)alkyl” refer to a heterocycle that is linked via a direct bond or alkyl group. Such groups include, for example, (4- to 10-membered heterocycle)C₀-C₄alkyl groups, in which the heterocycle contains from 4 to 10 ring members and is linked via a single covalent bond or C₁-C₄alkyl. Unless otherwise specified, the heterocycle portion of such groups may be saturated, partially saturated or aromatic. “(4- to 7-membered heterocycloalkyl)C₀-C₄alkyl” refers to a heterocycloalkyl group of from 4 to 7 ring members that is linked via a C₁-C₄alkyl.

Certain heterocycles are “heteroaryl” (i.e., groups that comprise at least one aromatic ring having from 1 to 4 heteroatoms). When the total number of S and 0 atoms in a heteroaryl group exceeds 1, then these heteroatoms are not adjacent to one another; preferably the total number of S and 0 atoms in a heteroaryl is not more than 1, 2 or 3, more preferably 1 or 2 and most preferably not more than 1. Examples of heteroaryl groups include pyridyl, furanyl, indolyl, pyrimidinyl, pyridizinyl, pyrazinyl, imidazolyl, oxazolyl, thienyl, thiazolyl, triazolyl, isoxazolyl, quinolinyl, pyrrolyl, pyrazolyl, benzodioxinyl and 5,6,7,8-tetrahydroisoquinolinyl. A (5- to 10-membered heteroaryl)C₀-C₄alkyl group is a heteroaryl group having fom 5 to 10 ring members and linked via a single covalent bond or a C₁-C₄alkyl group.

Other heterocycles are referred to herein as “heterocycloalkyl” (i.e., saturated or partially saturated heterocycles). Heterocycloalkyl groups have 1 or 2 rings, each with from 3 to about 8 ring atoms, and more typically from 5 to 7 ring atoms. Examples of heterocycloalkyl groups include morpholinyl, piperazinyl, piperidinyl and pyrrolidinyl.

Additional examples of heterocyclic groups include, but are not limited to, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl; 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

“A C5a receptor” is a G-protein coupled receptor that specifically binds C5a peptide. Certain preferred C5a receptors are human, such as the protein product of the sequence that produces the human C5a receptor PCR product described by Gerard and Gerard (1991) Nature 349:614-17. The human C5a receptor may also be that described by Boulay (1991) Biochemistry 30(12):2993-99 (nucleotide sequence encoding the receptor is available at GENBANK Accession No. M62505). Non-primate C5a receptors include the rat C5a receptor (encoded by the nucleotide sequence having GENBANK Accession No. X65862, Y09613 or AB003042), canine C5a receptor (encoded by the nucleotide sequence having GENBANK Accession No. X65860), and guinea pig C5a receptor (encoded by the nucleotide sequence having GENBANK Accession No. U86103).

A “C5a receptor modulator” (also referred to herein as a “modulator”) is any compound that modulates C5a receptor activation and/or activity (i.e., C5a receptor-mediated signal transduction, as measured using a C5a receptor-mediated chemotaxis, radioligand binding assay, or calcium mobilization assay as provided herein). In certain embodiments, such a modulator may be exhibit an affinity constant for binding to a C5a receptor of less than 1 micromolar in a standard C5a receptor radioligand binding assay; and/or an EC₅₀ of less than 1 micromolar in a standard C5a receptor-mediated chemotaxis assay or calcium mobilization assay. In other embodiments the a C5a receptor modulator may exhibit an affinity constant or EC₅₀ of less than 500 nM, 200 nM, 100 nM, 50 nM, 25 nM, 10 nM or 5 nM in such an assay. A modulator may be a C5a receptor agonist or antagonist, although, for certain purposes described herein, a modulator preferably inhibits C5a activation resulting from binding of C5a (i.e., the modulator is an antagonist). In addition, or alternatively, a modulator may act as an inverse agonist of C5a receptor. In certain embodiments, modulators provided herein modulate activation and/or activity of a primate C5a receptor, such as human C5a receptor, which may be a cloned, recombinantly expressed receptor or a naturally expressed receptor. For treating non-human animals of any particular species, a compound exhibiting high affinity for the C5a receptor of that particular species is preferred.

Certain C5a receptor modulators exhibit high activity in a standard in vitro C5a receptor mediated chemotaxis assay, as specified in Example 10, herein. Such compounds exhibit an EC₅₀ of 4 μM or less in such a standard C5a mediated chemotaxis assay, preferably an EC₅₀ of 1 μM or less in such an assay, more preferably an EC₅₀ of 0.1 μM or less in such an assay, and even more preferably and EC₅₀ of 10 nM or less in such an assay.

An “inverse agonist” of a C5a receptor is a compound that reduces the activity of the C5a receptor below its basal activity level in the absence of added C5a. Inverse agonists may also inhibit the activity of C5a at the C5a receptor, and/or may inhibit binding of C5a to the C5a receptor. The ability of a compound to inhibit the binding of C5a to the C5a receptor may be measured by a binding assay, such as the radioligand binding assay given in Example 15. The basal activity of the C5a receptor may be determined from a GTP binding assay, such as the assay of Example 16. The reduction of C5a receptor activity may also be determined from a GTP binding assay or a calcium mobilization assay such as the assay of Example 17.

A “neutral antagonist of the C5a receptor is a compound which inhibits the activity of C5a at the C5a receptor, but does not significantly change the basal activity of the C5a receptor. Neutral antagonists of the C5a receptor may inhibit the binding of C5a to the C5a receptor.

A “partial agonist” of the C5a receptor elevates the activity of the C5a receptor above the basal activity level of the receptor in the absence of C5a, but does not elevate the activity of the C5a receptor to the level brought about by saturating levels of the natural agonist, C5a. Partial agonist compounds may inhibit the binding of C5a to the C5a receptor. Partial agonists of the C5a receptor usually elevate the activity of the C5a receptor, producing a level of elevation ranging from 5% to 90% of the activity level brought about by receptor-saturating concentrations of the natural agonist, C5a.

A “patient” is any individual treated with a C5a modulator as provided herein. Patients include humans, as well as other animals such as companion animals (e.g., dogs and cats) and livestock. Patients may be experiencing one or more symptoms of a condition responsive to C5a receptor modulation, or may be free of such symptom(s) (i.e., treatment may be prophylactic).

(Heterocycloalkyl)Methyl Azole Derivatives

As noted above, the present invention provides (heterocycloalkyl)methyl azole derivatives of Formula I and more particularly provides (heterocycloalkyl)methyl imidazole, (heterocycloalkyl)methyl oxazole, and (heterocycloalkyl)methyl thiazole derivatives of Formula I. In certain aspects, such compounds are C5a receptor modulators and may be used to alter C5a receptor activity in a variety of contexts, including in the treatment of patients suffering from diseases or disorders responsive to C5a receptor modulation, such as autoimmune disorders and inflammatory conditions. C5a receptor modulators may also be used within a variety of in vitro assays (e.g., assays for receptor activity), as probes for detection and localization of C5a receptor and as standards in assays of ligand binding and C5a receptor-mediated signal transduction.

In general, C5a receptor modulators provided herein detectably alter, preferably decrease, C5a receptor activation and/or signal transduction activity at submicromolar concentrations. Such an alteration in C5a receptor activity may be measured using a standard in vitro C5a receptor-mediated chemotaxis assay (Example 10), a C5a receptor-mediated calcium mobilization assay (Example 17) and/or a radioligand binding assay (Example 15). The present invention is based, in part, on the discovery that small molecules of Formula I act as antagonists and/or inverse agonists of C5a receptors.

In addition to the compounds and pharmaceutically acceptable salts of Formula I described above, the present invention also provides compounds and pharmaceutically acceptable salts of Formula IA. Such compounds satisfy Formula I, but the variables carry the definitions set forth below:
A is oxygen, sulfur or NR;
R is C₁-C₇alkyl, C₂-C₇alkenyl, C₂-C₇alkynyl, C₁-C₆haloalkyl, (C₃-C₁₀carbocycle)C₁-C₄alkyl or (4- to 7-membered heterocycloalkyl)C₁-C₄alkyl, each of which is substituted with from 0 to 3 substituents independently chosen from hydroxy, halogen, amino, cyano, oxo, C₁-C₄alkyl, C₁-C₄alkoxy and C₁-C₂alkoxycarbonyl;
x is 0, 1 or 2;
J, K and each occurrence of L are chosen from oxygen, sulfur, NH and CH₂; such that no more than one of J, K and L is chosen from oxygen, sulfur and NH;
R₁ is chosen from:
   i) hydrogen, hydroxy, halogen, amino, cyano, nitro, —CHO, —CONH₂, C₁-C₆haloalkyl and C₁-C₆haloalkoxy;
   ii) C₁-C₆alkyl, C₂-C₇alkenyl, C₂-C₇alkynyl, C₂-C₆alkanoyl, C₁-C₆alkoxy, (C₃-C₇cycloalkyl)C₀-C₄alkyl, (4- to 10-membered heterocycloalkyl)C₀-C₄alkyl, mono- and di-(C₁-C₆alkyl)aminoC₀-C₆alkyl, mono- and di-(C₁-C₆alkyl)carboxamide, C₁-C₆alkoxycarbonyl, —SOₙ(C₁-C₆alkyl), —NHSOₙC₁-C₆alkyl, —(C₀-C₆alkyl)SOₙ(C₁-C₆alkyl), —SOₙN(C₁-C₆alkyl)(C₁-C₆alkyl), and —SOₙ-phenyl, wherein each n is independently 0, 1 or 2, and each of which is substituted with from 0 to 3 substituents independently chosen from hydroxy, halogen, amino, cyano, oxo, C₁-C₄alkyl, C₁-C₄alkoxy and C₁-C₂alkoxycarbonyl; and
   iii) naphthyl, phenyl and 5- and 6-membered heteroaryl, each of which is substituted with from 0 to 3 substituents independently chosen from R₁₁;
R₂ and R₃ are independently hydrogen or C₁-C₆alkyl;
R₄ represents 1 substituent chosen from:
   i) C₁-C₆alkyl, C₂-C₆alkenyl, C₂-C₆alkynyl, C₁-C₆alkoxycarbonyl, (C₃-C₇cycloalkyl)C₀-C₄alkyl and hexahydro-1,3-benzodioxolyl;
   ii) aryl having 1 ring or 2 fused or pendant rings;
   iii) (4- to 10-membered heterocycloalkyl)C₀-C₄alkyl;
   iv) phenyl fused to a 5- to 7-membered saturated or partially unsaturated ring that (a) has 0, 1 or 2 ring atoms independently chosen from N, O and S, with remaining ring atoms being carbon, and (b) is substituted with from 0 to 3 substituents independently chosen from halogen, C₁-C₈alkyl, C₁-C₈alkoxy, C₁-C₈haloalkyl, C₁-C₈haloalkoxy;
   v) (5- to 10-membered heteroaryl)C₀-C₄alkyl, having 1 ring or 2 fused or pendant rings, from 5 to 7 members in each ring, and in at least one ring from 1 to 3 heteroatoms independently selected from N, O, and S, wherein R₄ is not pyrimidyl; and
   vi) groups that are taken together with an R₅ moiety to form a fused phenyl or pyridyl ring;
   wherein each of i), ii), iii), iv), v) and vi) is substituted with from 0 to 3 substituents independently chosen from R₁₁;
R₅ represents from 0 to 3 substituents independently chosen from hydroxy, halogen, amino, cyano, nitro, —CHO, —CONH₂, C₁-C₂haloalkyl, C₁-C₂haloalkoxy, C₁-C₆alkyl, C₁-C₆alkoxy, C₃-C₇cycloalkylC₀-C₄alkyl, mono- and di-(C₁-C₆alkyl)aminoC₀-C₆alkyl, optionally substituted phenyl, and groups that are taken together with R₄ to form a fused, optionally substituted phenyl or pyridyl ring; and
Ar₁ represents
   i) phenyl or naphthyl, each of which is substituted with from 0 to 3 substituents independently chosen from amino, cyano, C₁-C₄alkyl, C₁-C₄alkoxy, C₁-C₂haloalkyl, C₁-C₂haloalkoxy, —COOH, —CONH₂, mono- and di-(C₁-C₄alkyl)amino, C₂-C₄alkanoyl, C₁-C₄sulfonate, C₁-C₄alkylsulfonyl, C₁-C₄alkylsulfinyl, C₁-C₄alkylthio, C₃-C₆alkanone, C₂-C₄alkyl ether, C₂-C₄alkanoyloxy, C₁-C₄alkoxycarbonyl and C₁-C₆alkylcarboxamide;
   ii) phenyl fused to a 5- to 7-membered saturated or partially unsaturated ring that (a) has 0, 1 or 2 ring atoms independently chosen from N, O and S, with remaining ring atoms being carbon, and (b) is substituted with from 0 to 3 substituents independently chosen from halogen, C₁-C₄alkyl, C₁-C₄alkoxy, C₁-C₂haloalkyl and C₁-C₂haloalkoxy; or
   iii) heteroaryl, having 1 ring or 2 fused or pendant rings, from 5 to 7 members in each ring, and in at least one ring from 1 to 3 heteroatoms independently selected from N, O, and S;
   wherein each of ii) and iii) is substituted with from 0 to 3 substituents independently chosen from R₁₁; and
R₁₁ is independently chosen at each occurrence from hydroxy, halogen, amino, cyano, C₁-C₆alkyl, C₁-C₆alkoxy, C₁-C₆haloalkyl, C₁-C₆haloalkoxy, —COOH, —CONH₂, —SO₂NH₂, mono- and di-(C₁-C₆alkyl)amino, C₂-C₆alkanoyl, C₁-C₆sulfonate, C₁-C₆alkylsulfonyl, C₁-C₆alkylsulfinyl, C₁-C₆alkylthio, C₃-C₆alkanone, C₂-C₆alkyl ether, C₂-C₆alkanoyloxy, C₁-C₆alkoxycarbonyl and C₁-C₆alkylcarboxamide.

Certain compounds of Formula IA further satisfy Formula IB, in which the variables are as described for Formula IA, except as set forth below:
R is chosen from C₁-C₇alkyl, C₂-C₇alkenyl, C₂-C₇alkynyl, (C₃-C₇cycloalkyl)C₁-C₄alkyl and (4- to 7-membered heterocycloalkyl)C₁-C₄alkyl, each of which is substituted with from 0 to 3 substituents independently chosen from hydroxy, halogen, amino, cyano, oxo, C₁-C₄alkyl, C₁-C₄alkoxy and C₁-C₂alkoxycarbonyl;
R₁ is chosen from:
   i) hydrogen, hydroxy, halogen, amino, cyano, nitro, —CHO, —CONH₂, C₁-C₆haloalkyl and C₁-C₆haloalkoxy;
   ii) C₁-C₆alkyl, C₁-C₆alkenyl, C₁-C₆alkynyl, C₁-C₆alkoxy, (C₃-C₇cycloalkyl)C₀-C₂alkyl, (4- to 10-membered heterocycloalkyl)C₀-C₂alkyl, and mono- and di-(C₁-C₆alkyl)carboxamide, each of which is substituted with from 0 to 3 substituents independently chosen from hydroxy, halogen, amino, cyano, oxo, C₁-C₄alkyl and C₁-C₄alkoxy, and
   iii) naphthyl, phenyl, pyridyl, thiazolyl, pyrimidinyl and thienyl, each of which is substituted with from 0 to 3 substituents independently chosen from hydroxy, halogen, amino, cyano, —COOH, —CONH₂, —SO₂NH₂, C₁-C₆haloalkyl, C₁-C₆haloalkoxy, C₁-C₆alkyl, C₁-C₆alkoxy, C₂-C₆alkanoyl, C₁-C₆alkylsulfonyl, C₁-C₆alkylsulfinyl, C₁-C₆alkylthio, C₃-C₆alkanone, C₂-C₆alkylether, C₂-C₆alkanoyloxy, C₁-C₆alkoxycarbonyl and C₁-C₆alkylcarboxamide;
R₄:
   i) represents C₁-C₆alkyl, C₂-C₇alkenyl, C₂-C₇alkynyl, C₁-C₆alkoxycarbonyl, (C₃-C₇cycloalkyl)C₀-C₄alkyl, hexahydro-1,3-benzodioxolyl, phenyl, naphthyl or (4- to 7-membered heterocycloalkyl)C₀-C₄alkyl, each of which is substituted with from 0 to 3 substituents independently chosen from hydroxy, halogen, amino, cyano, C₁-C₄alkyl, C₁-C₄alkoxy, C₁-C₂haloalkyl, C₁-C₂haloalkoxy, —COOH, —CONH₂, mono- and di-(C₁-C₄alkyl)amino, C₂-C₄alkanoyl, C₁-C₄sulfonate, C₁-C₄alkylsulfonyl, C₁-C₄alkylsulfinyl, C₁-C₄alkylthio, C₃-C₆alkanone, C₂-C₄alkyl ether, C₂-C₄alkanoyloxy, C₁-C₄alkoxycarbonyl, and C₁-C₆alkylcarboxamide; or
   ii) is phenyl fused to a 5- to 7-membered saturated or partially unsaturated ring that (a) has 0, 1 or 2 ring atoms independently chosen from N, O and S, with remaining ring atoms being carbon, and (b) is substituted with from 0 to 3 substituents independently chosen from halogen, C₁-C₄alkyl, C₁-C₄alkoxy, C₁-C₂haloalkyl and C₁-C₂haloalkoxy; or
   iii) is taken together with an R₅ moiety to form a fused phenyl or pyridyl ring that is substituted with from 0 to 3 substituents independently chosen from hydroxy, halogen, amino, cyano, C₁-C₄alkyl, C₁-C₄alkoxy, C₁-C₂haloalkyl, C₁-C₂haloalkoxy, —COOH, —CONH₂, and mono- and di-(C₁-C₄alkyl)amino;
R₅ represents from 0 to 3 substituents independently chosen from hydroxy, halogen, amino, cyano, C₁-C₄alkyl, C₁-C₄alkoxy, C₁-C₂haloalkyl, C₁-C₂haloalkoxy, —COOH, —CONH₂, mono- and di-(C₁-C₄alkyl)amino, and groups that are taken together with R₄ to form a fused, optionally substituted phenyl or pyridyl ring; and
Ar₁ represents phenyl, naphthyl, pyridyl, pyrimidinyl, pyridizinyl, pyrazinyl, pyrazolyl, imidazolyl, thiazolyl, isothiazolyl, pyrrolyl, oxazolyl, furanyl, indazolyl or thienyl, each of which is substituted with from 0 to 3 substituents independently chosen from amino, cyano, C₁-C₄alkyl, C₁-C₄alkoxy, C₁-C₂haloalkyl, C₁-C₂haloalkoxy, —COOH, —CONH₂, mono- and di-(C₁-C₄alkyl)amino, C₂-C₄alkanoyl, C₁-C₄sulfonate, C₁-C₄alkylsulfonyl, C₁-C₄alkylsulfinyl, C₁-C₄alkylthio, C₃-C₆alkanone, C₂-C₄alkyl ether, C₂-C₄alkanoyloxy, C₁-C₄alkoxycarbonyl and C₁-C₆alkylcarboxamide.

In certain embodiments, the present invention provides compounds and pharmaceutically acceptable salts of one or more of Formulas I, IA and IB in which A is oxygen; in other embodiments, A is sulfur; and in still further embodiments A is NR.

The present invention further provides compounds of Formulas II-XII shown below, in which the variables carry the definitions set forth above for Formula I, IA or IB:

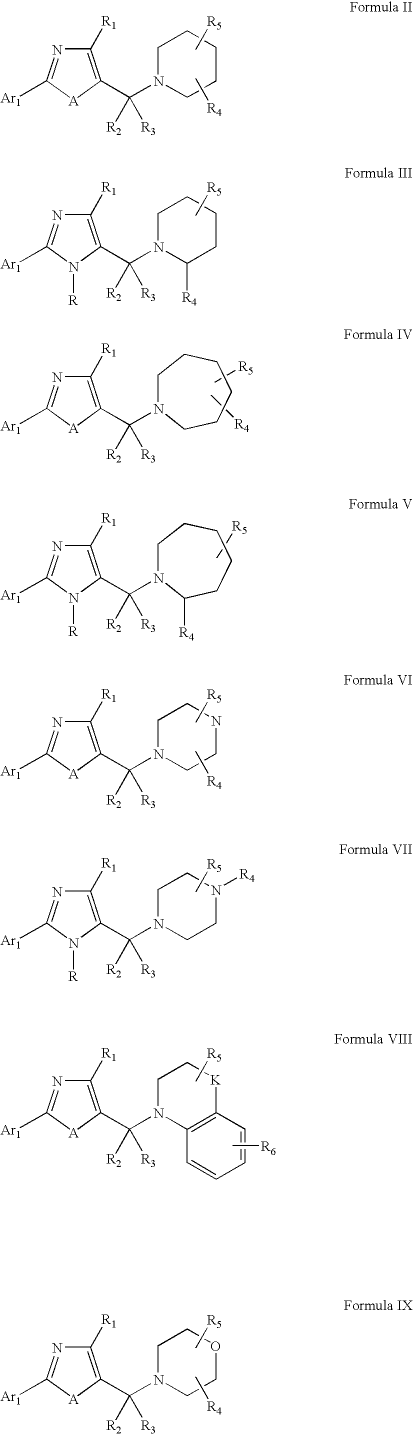

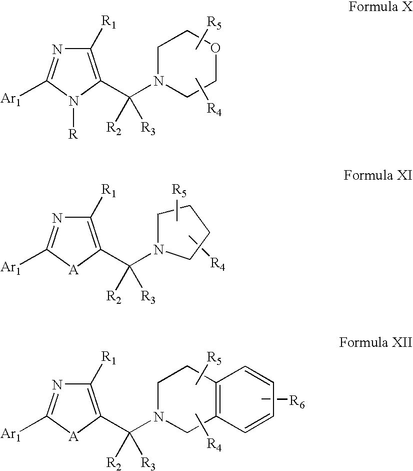

In Formula VIII, K is CH₂ or NR (e.g., NH). In certain embodiments of Formula VIII, A is NR and K is CH₂.

R₆, in Formula VIII and Formula XII, represents from 0 to 3 substituents independently chosen from hydroxy, halogen, amino, cyano, C₁C₄alkyl, C₁-C₄alkoxy, C₁-C₂haloalkyl, C₁-C₂haloalkoxy, —COOH, —CONH₂, and mono- and di-(C₁-C₄)alkylamino.

In certain embodiments of Formulas I, IA, IB and II-XII, one or more of the variables have one of the following definitions. It will be apparent the specific definitions provided below for each variable may be combined in any manner to produce an embodiment of the present invention. In addition, the definitions below are exemplary only, and do not limit the definitions for each variable recited above.

In certain embodiments, the variable R is:
(a) C₁-C₇alkyl, C₂-C₇alkenyl, (C₃-C₇cycloalkyl)C₁-C₄alkyl or (1,3-dioxylan-2-yl)C₁-C₄alkyl, each of which is substituted with from 0 to 3 substituents independently chosen from hydroxy, halogen, amino, cyano, oxo, C₁-C₄alkyl and C₁-C₄alkoxy; or
(b) C₁-C₅alkyl, C₂-C₄alkenyl or (1,3-dioxylan-2-yl)C₁-C₄alkyl, each of which is substituted with from 0 to 3 substituents independently chosen from hydroxy, halogen, amino, C₁-C₄alkyl and C₁-C₄alkoxy.

In certain embodiments, the variable R₁ is:
(a) chosen from i) halogen, ii) C₁-C₆alkyl, C₂-C₆alkenyl, C₁-C₆alkoxy, (C₃-C₇cycloalkyl)C₀-C₄alkyl, pyrrolidinylC₀-C₂alkyl, morpholinylC₀-C₂alkyl, piperinylC₀-C₂alkyl and piperazinylC₀-C₂alkyl, each of which is substituted with from 0 to 3 substituents independently chosen from hydroxy, halogen, amino, cyano, oxo, C₁-C₄alkyl and C₁-C₄alkoxy, and iii) phenyl or pyridyl, each of which is substituted with from 0 to 3 substituents independently chosen from hydroxy, halogen, amino, cyano, —COOH, —CONH₂, —SO₂NH₂, C₁-C₂haloalkyl, C₁-C₂haloalkoxy, C₁-C₆alkyl, C₁-C₆alkoxy, and mono- and di-C₁-C₄alkylamino;
(b) halogen, C₁-C₂alkyl, C₁-C₂alkoxy or pyrrolidinyl(C₁-C₂alkyl); or
(c) phenyl or pyridyl, each of which is substituted with from 0 to 3 substituents independently chosen from hydroxy, halogen, amino, cyano, —COOH, —CONH₂, —SO₂NH₂, C₁-C₂haloalkyl, C₁-C₂haloalkoxy, C₁-C₆alkyl, C₁-C₆alkoxy and mono- and di-C₁-C₄alkylamino.

In certain embodiments, the variables R₂ and R₃ are:
(a) independently hydrogen or C₁-C₃alkyl (e.g., hydrogen or methyl); or
(b) both hydrogen.

In certain embodiments, the variable R₄:
(a) represents 1 substituent chosen from C₁-C₆alkyl, C₁-C₆alkoxycarbonyl and C₃-C₇cycloalkyl, each of which is substituted with from 0 to 3 substituents independently chosen from hydroxy, halogen, amino, cyano, C₁-C₂alkyl, C₁-C₂alkoxy, C₁-C₂haloalkyl, C₁-C₂haloalkoxy, —COOH, —CONH₂ and mono- and di-(C₁-C₄alkyl)amino;
(b) represents 1 substituent chosen from phenyl, pyridylC₀-C₁alkyl, thienylC₀-C₁alkyl, naphthylC₀-C₁alkyl, indolylC₀-C₁alkyl, benzoxadiazolylC₀-C₁alkyl, benzoxazolylC₀-C₁alkyl, quinazolinylC₀-C₁alkyl, benzothiazolylC₀-C₁alkyl and benzimidazolylC₀-C₁alkyl, each of which is substituted with from 0 to 2 substituents independently chosen from hydroxy, halogen, amino, cyano, C₁-C₂alkyl, C₁-C₂alkoxy, C₁-C₂haloalkyl, C₁-C₂haloalkoxy, and mono- and di-(C₁-C₂)alkylamino;
(c) represents 1 substituent chosen from phenyl and pyridyl, each of which is substituted with from 0 to 3 substituents independently chosen from hydroxy, halogen, amino, cyano, C₁-C₄alkyl, C₁-C₄alkoxy, C₁-C₂haloalkyl, C₁-C₂haloalkoxy, —COOH, —CONH₂, mono- and di-(C₁-C₄)alkylamino, C₂-C₄alkanoyl, C₁-C₄sulfonate, C₁-C₄alkylsulfonyl, C₁-C₄alkylsulfinyl, C₁-C₄alkylthio, C₃-C₆alkanone, C₂-C₄alkyl ether, C₂-C₄alkanoyloxy, C₁-C₄alkoxycarbonyl and C₁-C₆alkylcarboxamide (in certain such embodiments, R₄ is phenyl or pyridyl, each of which is substituted with from 0 to 3 substituents independently chosen from hydroxy, halogen, amino, cyano, C₁-C₄alkyl, C₁-C₂alkoxy, C₁-C₂haloalkyl, C₁-C₂haloalkoxy, —COOH, —CONH₂, mono- and di-(C₁-C₂)alkylamino, C₁-C₂alkoxycarbonyl and C₁-C₂alkylcarboxamide);
(d) represents phenyl fused to a 5- to 7-membered saturated or partially unsaturated ring that (i) has 0, 1 or 2 ring atoms independently chosen from N, O and S, with remaining ring atoms being carbon, and (ii) is substituted with from 0 to 3 substituents independently chosen from halogen, C₁-C₄alkyl, C₁-C₄alkoxy, C₁-C₂haloalkyl and C₁-C₂haloalkoxy—for example, in certain such embodiments, R₄ represents 1,3-benzodioxol-5-yl, 2,3-dihydro-1-benzofuran-6-yl, 2,3-dihydro-1-benzofuran-5-yl, 2,3-dihydro-1,4-benzodioxin-6-yl, chroman-6-yl, chroman-7-yl, 1,3-benzothiazolyl or 2,3-dihydroindol-5-yl, each of which is substituted with from 0 to 2 substituents independently selected from hydroxy, halogen, amino, cyano, oxo, C₁-C₂haloalkyl, C₁-C₂haloalkoxy, C₁-C₂alkyl, C₁-C₂alkoxy, and mono- and di-(C₁-C₂)alkylamino; or R₄ represents benzo[1,3]dioxol-5-yl or 2,3-dihydro-benzo[1,4]dioxin-6-yl, each of which is substituted with from 0 to 3 substituents independently chosen from halogen, C₁-C₂alkyl, C₁-C₂alkoxy, C₁-C₂haloalkyl and C₁-C₂haloalkoxy;
(e) is a salicyl amide, such as 3-hydroxy,4-(—(C═O)NH₂)-phenyl or 3-methoxy,4-(—(C═O)NH₂)-phenyl; or
(f) is taken together with R₅ to form a fused phenyl or pyridyl ring that is substituted with from 0 to 3 substituents independently chosen from hydroxy, halogen, amino, cyano, C₁-C₄alkyl, C₁-C₄alkoxy, C₁-C₂haloalkyl, C₁-C₂haloalkoxy, —COOH, —CONH₂, and mono- and di-(C₁-C₄)alkylamino.

In certain embodiments, the variable R₅ represents from 0 to 3 substituents independently chosen from hydroxy, halogen, C₁-C₂alkyl and C₁-C₂alkoxy.

In certain embodiments, the variable Ar₁ is:
(a) phenyl, pyridyl, indazolyl or thienyl, each of which is substituted with 0 to 3 substituents independently chosen from C₁-C₄alkyl, C₁-C₄alkoxy, C₁-C₂haloalkyl, C₁-C₂haloalkoxy, and mono- and di-C₁-C₂alkylamino; or
(b) phenyl or mono- or di-substituted phenyl (e.g., phenyl mono- or di-substituted with ethyl or methyl, such as a 2,6-disubstituted phenyl).

The present invention further provides compounds of Formula XIII-Formula XVI:

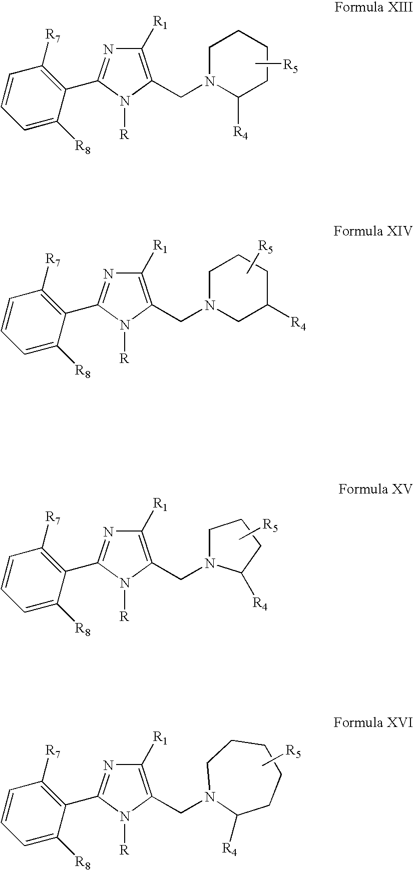

Within compounds of Formula XIII-Formula XVI:
R is chosen from ethyl, propyl, butyl, [1,3]dioxolan-2-ylmethyl and methoxymethyl;
R₁ is chosen from chloro, bromo, pyridyl and phenyl substituted with from 0 to 2 substituents independently chosen from halogen, C₁-C₂alkyl C₁-C₂alkxoy, hydroxymethyl, trifluoromethyl and trifluoromethoxy;
R₄ is chosen from phenyl and pyridyl, each of which is substituted with from 0 to 3 substituents independently chosen from halogen, hydroxy, —CONH₂, C₁-C₂alkyl, C₁-C₂alkoxy, and C₁-C₂alkoxycarbonyl; In certain embodiments, R₄ is chosen from 2,3-dimethoxyphenyl, 3,4-dimethoxyphenyl, pyrid-3-yl, 2-methoxy-phenyl, 3-methoxy-phenyl, 4-methoxy-phenyl, 2,3-dimethyl-4-methoxy-phenyl, 4-carboxamino-3-hydroxyphenyl, and 4-(CH₃—O(C═O))-3-hydroxy-phenyl;
R₅ represents from 0 to 2 substituents independently chosen from halogen, oxo, C₁-C₂alkyl and C₁-C₂alkoxy; In certain embodiments, R₅ represents 0 or 1 substituent chosen from methyl and oxo; and
R₇ and R₈ are independently chosen from hydrogen, methyl, methoxy, ethyl and ethoxy.

The present invention further provides compounds and pharmaceutically acceptable salts of Formula XVII-Formula XXIII:

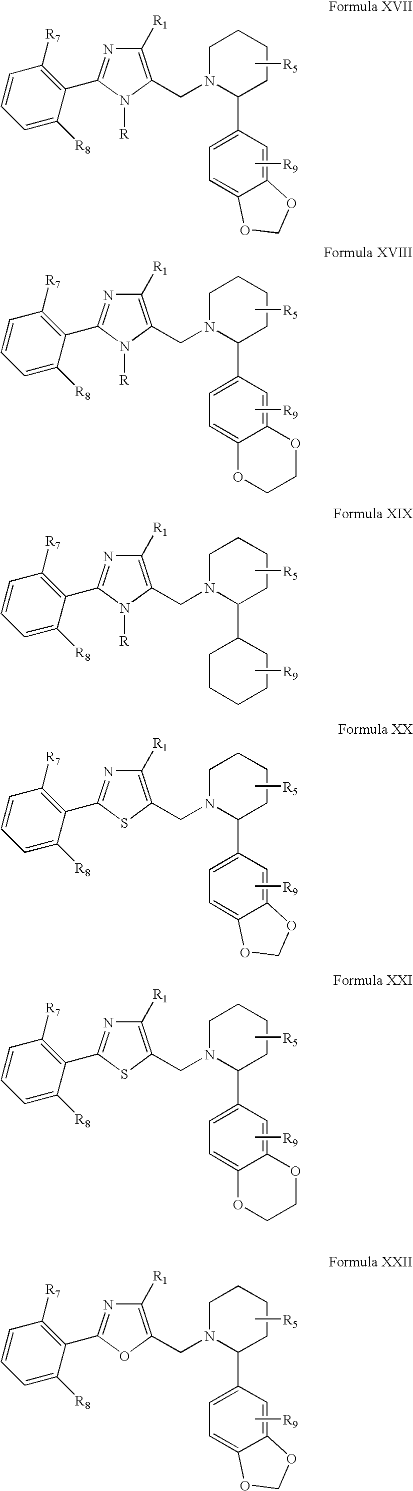

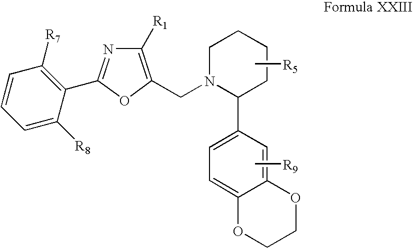

Within Formula XVII-Formula XXIII:
R is chosen from ethyl, propyl, butyl, [1,3]dioxolan-2-ylmethyl and methoxymethyl;
R₁ is chosen from chloro, bromo, pyridyl and phenyl substituted with from 0 to 2 substituents independently chosen from halogen, C₁-C₂alkyl, C₁-C₂alkxoy, hydroxymethyl, trifluoromethyl and trifluoromethoxy;
R₅ and R₉ each independently represent from 0 to 2 substituents independently chosen from halogen, oxo, C₁-C₂alkyl, and C₁-C₂alkoxy—in certain embodiments, R₅ and R₉ are both absent;
R₇ and R₈ are independently chosen from hydrogen, methyl, methoxy, ethyl and ethoxy.

The present invention further provides compounds and pharmaceutically acceptable salts of Formula XXIV:

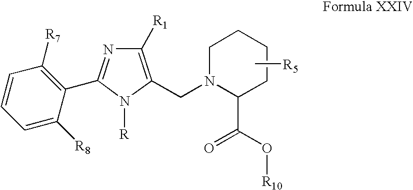

Within Formula XXIV:
R is chosen from ethyl, propyl, butyl, [1,3]dioxolan-2-ylmethyl and methoxymethyl;
R₁ is chosen from chloro, bromo, pyridyl and phenyl substituted with from 0 to 2 substituents independently chosen from halogen, C₁-C₂alkyl, C₁-C₂alkxoy, hydroxymethyl, trifluoromethyl and trifluoromethoxy;
R₅ represents from 0 to 2 substituents independently chosen from halogen, oxo, C₁-C₂alkyl and C₁-C₂alkoxy—in certain embodiments, R₅ is absent;
R₇ and R₈ are independently chosen from hydrogen, methyl, methoxy, ethyl and ethoxy; and
R₁₀ is methyl, ethyl or propyl.

The present invention further provides compounds and pharmaceutically acceptable salts of Formula XXV:

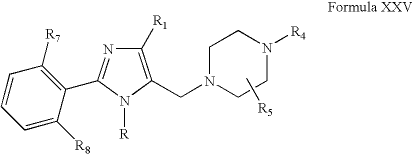

Within Formula XXV:
R is chosen from ethyl, propyl, butyl, [1,3]dioxolan-2-ylmethyl and methoxymethyl;
R₁ is chosen from chloro, bromo, pyridyl and phenyl substituted with from 0 to 2 substituents independently chosen from halogen, C₁-C₂alkyl, C₁-C₂alkoxy, hydroxymethyl, trifluoromethyl, and trifluoromethoxy;
R₄ is phenyl or benzyl, substituted with from 0 to 3 substituents independently chosen from halogen; in certain embodiments, R₄ is 2-fluoro-benzyl or 2,3-dimethyl-4-methoxy-benzyl.
R₅ represents from 0 to 2 substituents independently chosen from halogen, oxo, C₁-C₂alkyl, C₁-C₂alkoxy and phenyl—in certain embodiments, R₅ represents 0 or 1 substituent; and
R₇ and R₈ are independently chosen from hydrogen, methyl, methoxy, ethyl and ethoxy.

The present invention further provides compounds and pharmaceutically acceptable salts of Formula XXVI-Formula XXVII:

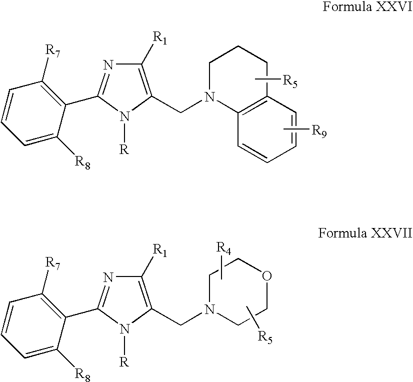

Within Formula XXVI and Formula XXVII:
R is chosen from ethyl, propyl, butyl, [1,3]dioxolan-2-ylmethyl and methoxymethyl;
R₁ is chosen from chloro, bromo, pyridyl and phenyl substituted with from 0 to 2 substituents independently chosen from halogen, C₁-C₂alkyl, C₁-C₂alkxoy, hydroxymethyl, trifluoromethyl and trifluoromethoxy;
R₄ is methyl or phenyl;
R₅ and R₉ represent from 0 to 2 substituents independently chosen from halogen, oxo, C₁-C₂alkyl, C₁-C₂alkoxy and phenyl—in certain embodiments, R₅ represents 0 or 1 substituent; and
R₇ and R₈ are independently chosen from hydrogen, methyl, methoxy, ethyl, and ethoxy.

The present invention further provides compounds and pharmaceutically acceptable salts of Formula XXVIII:

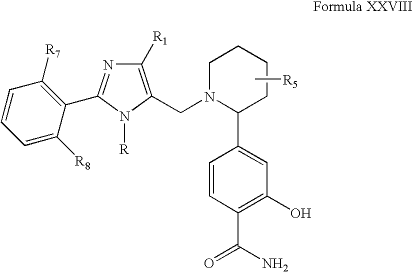

Within Formula XXVIII:
R is chosen from ethyl, propyl, butyl, and [1,3]dioxolan-2-ylmethyl and methoxymethyl;
R₁ is chosen from chloro, bromo, pyridyl, and phenyl substituted with from 0 to 2 substituents independently chosen from halogen, C₁-C₂alkyl, C₁-C₂alkxoy, hydroxymethyl, trifluoromethyl and trifluoromethoxy;
R₄ is methyl or phenyl;
R₅ represents from 0 to 2 substituents chosen from halogen, oxo, C₁-C₂alkyl, C₁-C₂alkoxy and phenyl—in certain embodiments, R₅ represents 0 or 1 substituent; and
R₇ and R₈ are independently chosen from hydrogen, methyl, methoxy, ethyl, and ethoxy.

Certain compounds of Formula I (and the other Formulas provided herein) have one or more stereogenic centers. In certain embodiments, such compounds may be enantiomers, and may have an enantiomeric excess of at least 55%. Within further embodiments, such compounds have an enantiomeric excess of at least 60%, 70%, 80%, 85%, 90%, 95%, 98%, or 99%. Certain compounds having one or more stereogenic centers have a enantiomeric excess of at least 99%.

Certain compounds of Formula I (and the other Formulas provided herein) have two or more stereogenic centers. In certain embodiments, such compounds have a diastereomeric excess of at least 55%. In other embodiments, such compounds have a diastereomeric excess of 60%, 70%, 80%, 85%, 90%, 95%, or 98%. Certain compounds having two or more stereogenic centers have a diastereomeric excess of at least 99%.

(Heterocycloalkyl)methyl azole derivatives provided herein detectably alter (modulate) C5a receptor activity and/or ligand binding, as determined using a standard in vitro C5 receptor-mediated chemotaxis assay (described in Example 10), radioligand binding assay (described in Example 15), or C5a receptor-mediated calcium mobilization assay (described in Example 17). Preferred (heterocycloalkyl)methyl azole derivatives exhibit an IC₅₀ of about 500 nM or less in such a standard C5a receptor-mediated chemotaxis, radioligand binding, and/or calcium mobilization assay, more preferably an IC₅₀ of about 250 nM or less in such an assay, still more preferably an IC₅₀ of about 200, 150, 100, 50, 25, 10, or 5 nM or less in such an assay.

Initial characterization of compounds can be conveniently carried out using a C5a receptor binding assay or functional assay, such as set forth in the Examples, and may be expedited by applying such assays in a high throughput screening setting. Additional assays suitable for determining the effects of small molecule compounds on C5a receptor binding and receptor modulatory activity, as well as assays suitable for measuring their effects on C5a-induced neutropenia in vivo, can be found in the published literature, for example in U.S. Pat. No. 5,807,824, which is incorporated herein by reference for its disclosure in this regard in Examples 6-9, columns 19-23, as well as for its discussion of complement and inflammation at columns 1-2. Those of skill in the art will recognize that such assays can be readily adapted to the use of cells or animals of different species as deemed appropriate.

In certain embodiments, preferred compounds have favorable pharmacological properties, including oral bioavailability (such that a sub-lethal or preferably a pharmaceutically acceptable oral dose, preferably less than 2 grams, more preferably of less than or equal to one gram, can provide a detectable in vivo effect such as a reduction of C5a-induced neutropenia), ability to inhibit leukocyte chemotaxis at nanomolar concentrations and preferably at sub-nanomolar concentrations, low toxicity (a preferred compound is nontoxic when a C5a receptor-modulatory amount is administered to a subject), minimal side effects (a preferred compound produces side effects comparable to placebo when a C5a receptor-modulatory amount of the compound is administered to a subject), low serum protein binding, and a suitable in vitro and in vivo half-life (a preferred compound exhibits an in vitro half-life that is equal to an in vivo half-life allowing for Q.I.D. dosing, preferably T.I.D. dosing, more preferably B.I.D. dosing, and most preferably once-a-day dosing). Distribution in the body to sites of complement activity is also desirable (e.g., compounds used to treat CNS disorders will preferably penetrate the blood brain barrier, while low brain levels of compounds used to treat periphereal disorders are typically preferred).

Routine assays that are well known in the art may be used to assess these properties, and identify superior compounds for a particular use. For example, assays used to predict bioavailability include transport across human intestinal cell monolayers, such as Caco-2 cell monolayers. Penetration of the blood brain barrier of a compound in humans may be predicted from the brain levels of the compound in laboratory animals given the compound (e.g., intravenously). Serum protein binding may be predicted from albumin binding assays, such as those described by Oravcova, et al. (1996) Journal of Chromatography B 677:1-27. Compound half-life is inversely proportional to the frequency of dosage of a compound required to achieve an C5a receptor modulatory amount. In vitro half-lives of compounds may be predicted from assays of microsomal half-life as described by Kuhnz and Gieschen (1998) Drug Metabolism and Disposition 26:1120-27.

As noted above, preferred compounds provided herein are nontoxic. In general, the term “nontoxic” as used herein shall be understood in a relative sense and is intended to refer to any substance that has been approved by the United States Food and Drug Administration (“FDA”) for administration to mammals (preferably humans) or, in keeping with established criteria, is susceptible to approval by the FDA for administration to mammals (preferably humans). In addition, a highly preferred nontoxic compound generally satisfies one or more of the following criteria: (1) does not substantially inhibit cellular ATP production; (2) does not significantly prolong heart QT intervals; (3) does not cause substantial liver enlargement, and (4) does not cause substantial release of liver enzymes.

As used herein, a compound that “does not substantially inhibit cellular ATP production” is a compound that satisfies the criteria set forth in Example 19, herein. In other words, cells treated as described in Example 19 with 100 μM of such a compound exhibit ATP levels that are at least 50% of the ATP levels detected in untreated cells. In more highly preferred embodiments, such cells exhibit ATP levels that are at least 80% of the ATP levels detected in untreated cells.

A compound that “does not significantly prolong heart QT intervals” is a compound that does not result in a statistically significant prolongation of heart QT intervals (as determined by electrocardiography) in guinea pigs, minipigs or dogs upon administration of twice the minimum dose yielding a therapeutically effective in vivo concentration. In certain preferred embodiments, a dose of 0.01, 0.05, 0.1, 0.5, 1, 5, 10, 40 or 50 mg/kg administered parenterally or orally does not result in a statistically significant prolongation of heart QT intervals. By “statistically significant” is meant results varying from control at the p<0.1 level or more preferably at the p<0.05 level of significance as measured using a standard parametric assay of statistical significance such as a student's T test.

A compound “does not cause substantial liver enlargement” if daily treatment of laboratory rodents (e.g., mice or rats) for 5-10 days with twice the minimum dose that yields a therapeutically effective in vivo concentration results in an increase in liver to body weight ratio that is no more than 100% over matched controls. In more highly preferred embodiments, such doses do not cause liver enlargement of more than 75% or 50% over matched controls. If non-rodent mammals (e.g., dogs) are used, such doses should not result in an increase of liver to body weight ratio of more than 50%, preferably not more than 25%, and more preferably not more than 10% over matched untreated controls. Preferred doses within such assays include 0.01, 0.05, 0.1, 0.5, 1, 5, 10, 40 or 50 mg/kg administered parenterally or orally.

Similarly, a compound “does not promote substantial release of liver enzymes” if administration of twice the minimum dose yielding a therapeutically effective in vivo concentration does not elevate serum levels of ALT, LDH or AST in laboratory rodents by more than 100% over matched mock-treated controls. In more highly preferred embodiments, such doses do not elevate such serum levels by more than 75% or 50% over matched controls. Alternately, a compound “does not promote substantial release of liver enzymes” if, in an in vitro hepatocyte assay, concentrations (in culture media or other such solutions that are contacted and incubated with hepatocytes in vitro) equivalent to two-fold the minimum in vivo therapeutic concentration of the compound do not cause detectable release of any of such liver enzymes into culture medium above baseline levels seen in media from matched mock-treated control cells. In more highly preferred embodiments, there is no detectable release of any of such liver enzymes into culture medium above baseline levels when such compound concentrations are five-fold, and preferably ten-fold the minimum in vivo therapeutic concentration of the compound.

In other embodiments, certain preferred compounds do not inhibit or induce microsomal cytochrome P450 enzyme activities, such as CYP1A2 activity, CYP2A6 activity, CYP2C9 activity, CYP2C19 activity, CYP2D6 activity, CYP2E1 activity or CYP3A4 activity at a concentration equal to the minimum therapeutically effective in vivo concentration.

Certain preferred compounds are not clastogenic or mutagenic (e.g., as determined using standard assays such as the Chinese hamster ovary cell vitro micronucleus assay, the mouse lymphoma assay, the human lymphocyte chromosomal aberration assay, the rodent bone marrow micronucleus assay, the Ames test or the like) at a concentration equal to the minimum therapeutically effective in vivo concentration. In other embodiments, certain preferred compounds do not induce sister chromatid exchange (e.g., in Chinese hamster ovary cells) at such concentrations.

In certain embodiments, preferred compounds exert their receptor-modulatory effects with high specificity. This means that they only bind to, activate, or inhibit the activity of certain receptors other than C5a receptors with affinity constants of greater than 100 nanomolar, preferably greater than 1 micromolar, more preferably greater than 4 micromolar. Also provided herein are highly specific C5a receptor modulatory compounds that exhibit 200-fold greater affinity for the C5a receptor that for other cellular receptors. Such receptors include neurotransmitter receptors such as alpha- or beta-adrenergic receptors, muscarinic receptors (particularly m1, m2 or m3 receptors), dopamine receptors, and metabotropic glutamate receptors; as well as histamine receptors and cytokine receptors (e.g., interleukin receptors, particularly IL-8 receptors). Such receptors may also include GABA_{A} receptors, bioactive peptide receptors (other than C5a receptors and C3a receptors, including NPY or VIP receptors), neurokinin receptors, bradykinin receptors, and hormone receptors (e.g., CRF receptors, thyrotropin releasing hormone receptors or melanin-concentrating hormone receptors). Compounds that act with high specificity generally exhibit fewer undesirable side effects.

Within certain embodiments, modulators provided herein do not bind detectably to receptors that do not mediate inflammatory responses, such as GABA receptors, MCH receptors, NPY receptors, dopamine receptors, serotonin receptors and VR₁ receptors, with high or even moderate affinity. In addition, or alternatively, certain preferred C5a receptor modulators exhibit an affinity for C5a receptor that is substantially higher than for receptors that do not mediate inflammatory responses (e.g., at least five times higher, at least ten times higher or at least 100 times higher). Assays for evaluating binding to receptors that do not mediate inflammatory responses include, for example, those described in U.S. Pat. No. 6,310,212, which is incorporated herein by reference for its disclosure of a GABA_{A} receptor binding assays in Example 14, columns 16-17, in U.S. patent application Ser. No. 10/152,189 which is incorporated herein by reference for its disclosure of an MCH receptor binding assay in Example 2, pages 104-105, in U.S. Pat. No. 6,362,186, which is incorporated herein by reference for its disclosure of CRF₁ and NPY receptor binding assays in Example 19, columns 45-46, in U.S. Pat. No. 6,355,644, which is incorporated herein by reference for its disclosure of a dopamine receptor binding assay at column 10, and in U.S. Pat. No. 6,482,611, which is incorporated herein by reference for its disclosure of VR₁ receptor binding assays in Examples 4-5, column 14. It will be apparent that the C5a receptor modulators provided herein may, but need not, bind to one or more other receptors known to mediate inflammatory responses, such as C3a receptors and/or A₃ receptors.

Certain preferred compounds are C5a receptor antagonists that do not possess significant (e.g., greater than 5%) agonist activity in any of the C5a receptor-mediated functional assays discussed herein. Specifically, this undesired agonist activity can be evaluated, for example, in the GTP binding assay of Example 16, by measuring small molecule mediated GTP binding in the absence of the natural agonist, C5a. Similarly, in a calcium mobilization assay (e.g., that of Example 17) a small molecule compound can be directly assayed for the ability of the compound to stimulate calcium levels in the absence of the natural agonist, C5a. The preferred extent of C5a agonist activity exhibited by compounds provided herein is less than 10%, 5% or 2% of the response elicited by the natural agonist, C5a.

Also preferred, in certain embodiments, are C5a receptor modulators that inhibit the occurrence of C5a-induced oxidative burst (OB) in inflammatory cells (e.g., neutrophil) as can be conveniently determined using an in vitro neutrophil OB assay.

For detection purposes, compounds provided herein may be isotopically-labeled or radiolabeled. Accordingly, compounds recited in Formula I (or any other formula specifically recited herein) may have one or more atoms replaced by an atom of the same element having an atomic mass or mass number different from the atomic mass or mass number usually found in nature. Examples of isotopes that can be present in compounds provided herein include isotopes of hydrogen, carbon, nitrogen, oxygen, phosphorous, fluorine and chlorine, such as ²H, ³H, ¹¹C, ¹³C, ¹⁴C, ¹⁵N, ¹⁸O, ¹⁷O, ³¹P, ³²P, ³⁵S, ¹⁸F and ³⁶Cl. In addition, substitution with heavy isotopes such as deuterium (i.e., ²H) can afford certain therapeutic advantages resulting from greater metabolic stability, for example increased in vivo half-life or reduced dosage requirements and, hence, may be preferred in some circumstances.

Pharmaceutical Compositions

The present invention also provides pharmaceutical compositions comprising one or more compounds provided herein, together with at least one physiologically acceptable carrier or excipient. Pharmaceutical compositions may comprise, for example, one or more of water, buffers (e.g., neutral buffered saline or phosphate buffered saline), ethanol, mineral oil, vegetable oil, dimethylsulfoxide, carbohydrates (e.g., glucose, mannose, sucrose or dextrans), mannitol, proteins, adjuvants, polypeptides or amino acids such as glycine, antioxidants, chelating agents such as EDTA or glutathione and/or preservatives. As noted above, other active ingredients may (but need not) be included in the pharmaceutical compositions provided herein.

Pharmaceutical compositions may be formulated for any appropriate manner of administration, including, for example, topical, oral, nasal, rectal or parenteral administration. The term parenteral as used herein includes subcutaneous, intradermal, intravascular (e.g., intravenous), intramuscular, spinal, intracranial, intrathecal and intraperitoneal injection, as well as any similar injection or infusion technique. In certain embodiments, compositions in a form suitable for oral use are preferred. Such forms include, for example, tablets, troches, lozenges, aqueous or oily suspensions, dispersible powders or granules, emulsion, hard or soft capsules, or syrups or elixirs. Within yet other embodiments, compositions provided herein may be formulated as a lyophilizate. Formulation for topical administration may be preferred for certain conditions (e.g., in the treatment of skin conditions such as burns or itch).

Compositions intended for oral use may further comprise one or more components such as sweetening agents, flavoring agents, coloring agents and/or preserving agents in order to provide appealing and palatable preparations. Tablets contain the active ingredient in admixture with physiologically acceptable excipients that are suitable for the manufacture of tablets. Such excipients include, for example, inert diluents (e.g., calcium carbonate, sodium carbonate, lactose, calcium phosphate or sodium phosphate), granulating and disintegrating agents (e.g., corn starch or alginic acid), binding agents (e.g., starch, gelatin or acacia) and lubricating agents (e.g., magnesium stearate, stearic acid or talc). The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent (e.g., calcium carbonate, calcium phosphate or kaolin), or as soft gelatin capsules wherein the active ingredient is mixed with water or an oil medium (e.g., peanut oil, liquid paraffin or olive oil).

Aqueous suspensions contain the active material(s) in admixture with excipients suitable for the manufacture of aqueous suspensions. Such excipients include suspending agents (e.g., sodium carboxymethylcellulose, methylcellulose, hydropropylmethylcellulose, sodium alginate, polyvinylpyrrolidone, gum tragacanth and gum acacia); and dispersing or wetting agents (e.g., naturally-occurring phosphatides such as lecithin, condensation products of an alkylene oxide with fatty acids such as polyoxyethylene stearate, condensation products of ethylene oxide with long chain aliphatic alcohols such as heptadecaethyleneoxycetanol, condensation products of ethylene oxide with partial esters derived from fatty acids and a hexitol such as polyoxyethylene sorbitol monooleate, or condensation products of ethylene oxide with partial esters derived from fatty acids and hexitol anhydrides such as polyethylene sorbitan monooleate). Aqueous suspensions may also comprise one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil (e.g., arachis oil, olive oil, sesame oil or coconut oil) or in a mineral oil such as liquid paraffin. The oily suspensions may contain a thickening agent such as beeswax, hard paraffin or cetyl alcohol. Sweetening agents such as those set forth above, and/or flavoring agents may be added to provide palatable oral preparations. Such suspensions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, such as sweetening, flavoring and coloring agents, may also be present.

Pharmaceutical compositions may also be in the form of oil-in-water emulsions. The oily phase may be a vegetable oil (e.g., olive oil or arachis oil), a mineral oil (e.g., liquid paraffin) or a mixture thereof. Suitable emulsifying agents include naturally-occurring gums (e.g., gum acacia or gum tragacanth), naturally-occurring phosphatides (e.g., soy bean lecithin, and esters or partial esters derived from fatty acids and hexitol), anhydrides (e.g., sorbitan monoleate) and condensation products of partial esters derived from fatty acids and hexitol with ethylene oxide (e.g., polyoxyethylene sorbitan monoleate). An emulsion may also comprise one or more sweetening and/or flavoring agents.

Syrups and elixirs may be formulated with sweetening agents, such as glycerol, propylene glycol, sorbitol or sucrose. Such formulations may also comprise one or more demulcents, preservatives, flavoring agents and/or coloring agents.

Formulations for topical administration typically comprise a topical vehicle combined with active agent(s), with or without additional optional components. Suitable topical vehicles and additional components are well known in the art, and it will be apparent that the choice of a vehicle will depend on the particular physical form and mode of delivery. Topical vehicles include water; organic solvents such as alcohols (e.g., ethanol or isopropyl alcohol) or glycerin; glycols (e.g., butylene, isoprene or propylene glycol); aliphatic alcohols (e.g., lanolin); mixtures of water and organic solvents and mixtures of organic solvents such as alcohol and glycerin; lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile); and hydrocarbon-based materials such as microsponges and polymer matrices. A composition may further include one or more components adapted to improve the stability or effectiveness of the applied formulation, such as stabilizing agents, suspending agents, emulsifying agents, viscosity adjusters, gelling agents, preservatives, antioxidants, skin penetration enhancers, moisturizers and sustained release materials. Examples of such components are described in Martindale—The Extra Pharmacopoeia (Pharmaceutical Press, London 1993) and Martin (ed.), Remington's Pharmaceutical Sciences. Formulations may comprise microcapsules, such as hydroxymethylcellulose or gelatin-microcapsules, liposomes, albumin microspheres, microemulsions, nanoparticles or nanocapsules.

A topical formulation may be prepared in a variety of physical forms including, for example, solids, pastes, creams, foams, lotions, gels, powders, aqueous liquids and emulsions. The physical appearance and viscosity of such forms can be governed by the presence and amount of emulsifier(s) and viscosity adjuster(s) present in the formulation. Solids are generally firm and non-pourable and commonly are formulated as bars or sticks, or in particulate form; solids can be opaque or transparent, and optionally can contain solvents, emulsifiers, moisturizers, emollients, fragrances, dyes/colorants, preservatives and other active ingredients that increase or enhance the efficacy of the final product. Creams and lotions are often similar to one another, differing mainly in their viscosity; both lotions and creams may be opaque, translucent or clear and often contain emulsifiers, solvents, and viscosity adjusting agents, as well as moisturizers, emollients, fragrances, dyes/colorants, preservatives and other active ingredients that increase or enhance the efficacy of the final product. Gels can be prepared with a range of viscosities, from thick or high viscosity to thin or low viscosity. These formulations, like those of lotions and creams, may also contain solvents, emulsifiers, moisturizers, emollients, fragrances, dyes/colorants, preservatives and other active ingredients that increase or enhance the efficacy of the final product. Liquids are thinner than creams, lotions, or gels and often do not contain emulsifiers. Liquid topical products often contain solvents, emulsifiers, moisturizers, emollients, fragrances, dyes/colorants, preservatives and other active ingredients that increase or enhance the efficacy of the final product.

Suitable emulsifiers for use in topical formulations include, but are not limited to, ionic emulsifiers, cetearyl alcohol, non-ionic emulsifiers like polyoxyethylene oleyl ether, PEG-40 stearate, ceteareth-12, ceteareth-20, ceteareth-30, ceteareth alcohol, PEG-100 stearate and glyceryl stearate. Suitable viscosity adjusting agents include, but are not limited to, protective colloids or non-ionic gums such as hydroxyethylcellulose, xanthan gum, magnesium aluminum silicate, silica, microcrystalline wax, beeswax, paraffin, and cetyl palmitate. A gel composition may be formed by the addition of a gelling agent such as chitosan, methyl cellulose, ethyl cellulose, polyvinyl alcohol, polyquaterniums, hydroxyethylcellulose, hydroxypropylcellulose, hydroxypropylmethylcellulose, carbomer or ammoniated glycyrrhizinate. Suitable surfactants include, but are not limited to, nonionic, amphoteric, ionic and anionic surfactants. For example, one or more of dimethicone copolyol, polysorbate 20, polysorbate 40, polysorbate 60, polysorbate 80, lauramide DEA, cocamide DEA, and cocamide MEA, oleyl betaine, cocamidopropyl phosphatidyl PG-dimonium chloride, and ammonium laureth sulfate may be used within topical formulations. Suitable preservatives include, but are not limited to, antimicrobials such as methylparaben, propylparaben, sorbic acid, benzoic acid, and formaldehyde, as well as physical stabilizers and antioxidants such as vitamin E, sodium ascorbate/ascorbic acid and propyl gallate. Suitable moisturizers include, but are not limited to, lactic acid and other hydroxy acids and their salts, glycerin, propylene glycol, and butylene glycol. Suitable emollients include lanolin alcohol, lanolin, lanolin derivatives, cholesterol, petrolatum, isostearyl neopentanoate and mineral oils. Suitable fragrances and colors include, but are not limited to, FD&C Red No. 40 and FD&C Yellow No. 5. Other suitable additional ingredients that may be included a topical formulation include, but are not limited to, abrasives, absorbents, anti-caking agents, anti-foaming agents, anti-static agents, astringents (e.g., witch hazel, alcohol and herbal extracts such as chamomile extract), binders/excipients, buffering agents, chelating agents, film forming agents, conditioning agents, propellants, opacifying agents, pH adjusters and protectants.

An example of a suitable topical vehicle for formulation of a gel is: hydroxypropylcellulose (2.1%); 70/30 isopropyl alcohol/water (90.9%); propylene glycol (5.1%); and Polysorbate 80 (1.9%). An example of a suitable topical vehicle for formulation as a foam is: cetyl alcohol (1.1%); stearyl alcohol (0.5%; Quaternium 52 (1.0%); propylene glycol (2.0%); Ethanol 95 PGF3 (61.05%); deionized water (30.05%); P75 hydrocarbon propellant (4.30%). All percents are by weight.

Typical modes of delivery for topical compositions include application using the fingers; application using a physical applicator such as a cloth, tissue, swab, stick or brush; spraying (including mist, aerosol or foam spraying); dropper application; sprinkling; soaking; and rinsing. Controlled release vehicles can also be used.

A pharmaceutical composition may be prepared as a sterile injectible aqueous or oleaginous suspension. The modulator, depending on the vehicle and concentration used, can either be suspended or dissolved in the vehicle. Such a composition may be formulated according to the known art using suitable dispersing, wetting agents and/or suspending agents such as those mentioned above. Among the acceptable vehicles and solvents that may be employed are water, 1,3-butanediol, Ringer's solution and isotonic sodium chloride solution. In addition, sterile, fixed oils may be employed as a solvent or suspending medium. For this purpose any bland fixed oil may be employed, including synthetic mono- or diglycerides. In addition, fatty acids such as oleic acid find use in the preparation of injectible compositions, and adjuvants such as local anesthetics, preservatives and/or buffering agents can be dissolved in the vehicle.

C5a modulators described herein may be formulated as inhaled formulations, including sprays, mists, or aerosols. Such formulations are particularly useful for the treatment of asthma or other respiratory conditions. For inhalation formulations, the compounds provided herein may be delivered via any inhalation methods known to those skilled in the art. Such inhalation methods and devices include, but are not limited to, metered dose inhalers with propellants such as CFC or HFA or propellants that are physiologically and environmentally acceptable. Other suitable devices are breath operated inhalers, multidose dry powder inhalers and aerosol nebulizers. Aerosol formulations for use in the subject method typically include propellants, surfactants and co-solvents and may be filled into conventional aerosol containers that are closed by a suitable metering valve.

Inhalant compositions may comprise liquid or powdered compositions containing the active ingredient that are suitable for nebulization and intrabronchial use, or aerosol compositions administered via an aerosol unit dispensing metered doses. Suitable liquid compositions comprise the active ingredient in an aqueous, pharmaceutically acceptable inhalant solvent, e.g., isotonic saline or bacteriostatic water. The solutions are administered by means of a pump or squeeze-actuated nebulized spray dispenser, or by any other conventional means for causing or enabling the requisite dosage amount of the liquid composition to be inhaled into the patient's lungs. Suitable formulations, wherein the carrier is a liquid, for administration, as for example, a nasal spray or as nasal drops, include aqueous or oily solutions of the active ingredient.

Formulations suitable for nasal administration, wherein the carrier is a solid, include a coarse powder having a particle size, for example, in the range of 20 to 500 microns which is administered in the manner in which snuff is administered (i.e., by rapid inhalation through the nasal passage from a container of the powder held close up to the nose). Suitable powder compositions include, by way of illustration, powdered preparations of the active ingredient thoroughly intermixed with lactose or other inert powders acceptable for intrabronchial administration. The powder compositions can be administered via an aerosol dispenser or encased in a breakable capsule which may be inserted by the patient into a device that punctures the capsule and blows the powder out in a steady stream suitable for inhalation.

Modulators may also be prepared in the form of suppositories (e.g., for rectal administration). Such compositions can be prepared by mixing the drug with a suitable non-irritating excipient that is solid at ordinary temperatures but liquid at the rectal temperature and will therefore melt in the rectum to release the drug. Suitable excipients include, for example, cocoa butter and polyethylene glycols.

Pharmaceutical compositions may be formulated as sustained release formulations (i.e., a formulation such as a capsule that effects a slow release of modulator following administration). Such formulations may generally be prepared using well known technology and administered by, for example, oral, rectal or subcutaneous implantation, or by implantation at the desired target site. Carriers for use within such formulations are biocompatible, and may also be biodegradable; preferably the formulation provides a relatively constant level of modulator release. The amount of modulator contained within a sustained release formulation depends upon, for example, the site of implantation, the rate and expected duration of release and the nature of the condition to be treated or prevented.

In addition to or together with the above modes of administration, a modulator may be conveniently added to food or drinking water (e.g., for administration to non-human animals including companion animals (such as dogs and cats) and livestock). Animal feed and drinking water compositions may be formulated so that the animal takes in an appropriate quantity of the composition along with its diet. It may also be convenient to present the composition as a premix for addition to feed or drinking water.

Modulators are generally administered in a therapeutically effective amount. Preferred systemic doses range from about 0.1 mg to about 140 mg per kilogram of body weight per day (about 0.5 mg to about 7 g per patient per day), with oral doses generally being about 5-20 fold higher than intravenous doses. The amount of active ingredient that may be combined with the carrier materials to produce a single dosage form will vary depending upon the host treated and the particular mode of administration. Dosage unit forms will generally contain between from about 1 mg to about 500 mg of an active ingredient.

Packaged pharmaceutical compositions are also provided herein, comprising a C5a receptor modulatory amount of at least one C5a receptor antagonist in a container (preferably sealed) and instructions for using the C5a receptor antagonist to treat a condition responsive to C5a receptor modulation (e.g., rheumatoid arthritis, osteoarthritis, psoriasis, cardiovascular disease, reperfusion injury, bronchial asthma and other allergic conditions, chronic pulmonary obstructive disorder (COPD), fibrosis, cystic fibrosis, Alzheimer's disease, inflammatory bowel disease, stroke, myocardial infarction, atherosclerosis, ischemic heart disease or ischemia-reperfusion injury). The active agent(s) may be formulated for administration in a single pharmaceutical preparation (e.g., within the same pharmaceutical composition). Alternatively, each of the active agents may be formulated for separate administration, by the same or different routes of administration. Within a packaged pharmaceutical preparation, a C5a receptor modulatory amount may be packaged as a single dose unit; alternatively, multiple doses may be packaged together for convenience. The C5a receptor modulator may be presented in any suitable container including, but not limited to, a plastic, paper, metal or glass package such as an ampoule, bottle, vial, blister package, infusion bag, syringe, inhaler or tube. For example, a packaged pharmaceutical preparation for oral administration of an active agent may comprise a blister package containing rows of tablets. Instructions may be present on a label attached to the container or on exterior packaging, or may be provided as a package insert.

Methods of Use

C5a modulators provided herein may be used as agonists or (preferably) antagonists, such as inverse agonists, of C5a receptors in a variety of contexts, both in vitro and in vivo. Within certain aspects, C5a antagonists may be used to inhibit the binding of C5a receptor ligand (e.g., C5a) to C5a receptor in vitro or in vivo. In general, such methods comprise the step of contacting a C5a receptor with a sufficient concentration of one or more C5a receptor modulators as provided herein, in the presence of C5a receptor ligand in aqueous solution and under conditions otherwise suitable for binding of the ligand to C5a receptor. The C5a receptor may be present in suspension (e.g., in an isolated membrane or cell preparation), or in a cultured or isolated cell. Within certain embodiments, the C5a receptor is expressed by a cell present in a patient, and the aqueous solution is a body fluid. In general, the concentration of C5a receptor modulator contacted with the receptor should be sufficient to inhibit C5a binding to C5a receptor in vitro as measured, for example, using a calcium mobilization assay or chemotaxis assay as described herein.

Also provided herein are methods for modulating, preferably inhibiting, the signal-transducing activity of a C5a receptor. Such modulation may be achieved by contacting a C5a receptor (either in vitro or in vivo) with a C5a receptor modulatory amount of one or more C5a receptor modulators provided herein under conditions suitable for binding of the modulator(s) to the receptor. The receptor may be present in solution or suspension, in a cultured or isolated cell preparation or within a patient. Modulation of signal transducing activity may be assessed by detecting an effect on calcium ion conductance (also referred to as calcium mobilization or flux) or by detecting an effect on C5a receptor-mediated cellular chemotaxis. C5a receptor modulator(s) provided herein are preferably administered to a patient (e.g., a human) orally or topically, and are present within at least one body fluid of the animal while modulating C5a receptor signal-transducing activity.

The present invention further provides methods for treating patients suffering from conditions responsive to C5a receptor modulation. As used herein, the term “treatment” encompasses both disease-modifying treatment and symptomatic treatment, either of which may be prophylactic (i.e., before the onset of symptoms, in order to prevent, delay or reduce the severity of symptoms) or therapeutic (i.e., after the onset of symptoms, in order to reduce the severity and/or duration of symptoms). A condition is “responsive to C5a receptor modulation” if modulation of C5a receptor activity results in alleviation of the condition or a symptom thereof. Patients may include primates (especially humans), domesticated companion animals (such as dogs, cats, horses) and livestock (such as cattle, pigs, sheep), with dosages as described herein.

Conditions that are responsive to C5a receptor modulation include the following:

Autoimmune disorders—e.g., rheumatoid arthritis, systemic lupus erythematosus (and associated glomerulonephritis), psoriasis, Crohn's disease, vasculitis, irritable bowel syndrome, inflammatory bowel disease, osteoartiritis, dermatomyositis, multiple sclerosis, bronchial asthma and other allergic conditions, pemphigus, pemphigoid, scleroderma, myasthenia gravis, autoimmune hemolytic and thrombocytopenic states, Goodpasture's syndrome (and associated glomerulonephritis and pulmonary hemorrhage), immunovasculitis, tissue graft rejection, and hyperacute rejection of transplanted organs.

For asthma therapy, C5a receptor antagonists provided herein may be used to prevent or decrease the severity of both acute early phase asthma attack and the late phase reactions that follow such an asthma attack.

Inflammatory disorders and related conditions—e.g., neutropenia, sepsis, septic shock, Alzheimer's disease, stroke, inflammation associated with severe burns, lung injury, and ischemia-reperfusion injury, osteoarthritis, as well as acute (adult) respiratory distress syndrome (ARDS), chronic pulmonary obstructive disorder (COPD), systemic inflammatory response syndrome (SIRS), fibrosis, cystic fibrosis, and multiple organ dysfunction syndrome (MODS). Also included are pathologic sequellae associated with insulin-dependent diabetes mellitus (including diabetic retinopathy), lupus nephropathy, Heyman nephritis, membranous nephritis and other forms of glomerulonephritis, contact sensitivity responses, and inflammation resulting from contact of blood with artificial surfaces that can cause complement activation, as occurs, for example, during extracorporeal circulation of blood (e.g., during hemodialysis or via a heart-lung machine, for example, in association with vascular surgery such as coronary artery bypass grafting or heart valve replacement) such as extracorporeal post-dialysis syndrome, or in association with contact with other artificial vessel or container surfaces (e.g., ventricular assist devices, artificial heart machines, transfusion tubing, blood storage bags, plasmapheresis, plateletpheresis, and the like).

Cardiovascular and Cerebrovascular Disorders—e.g., myocardial infarction, coronary thrombosis, vascular occlusion, post-surgical vascular reocclusion, atherosclerosis, traumatic central nervous system injury, and ischemic heart disease. For example, a C5a receptor modulatory amount of a compound provided herein may be administered to a patient at risk for myocardial infarction or thrombosis (i.e., a patient who has one or more recognized risk factor for myocardial infarction or thrombosis, such as, but not limited to, obesity, smoking, high blood pressure, hypercholesterolemia, previous or genetic history of myocardial infarction or thrombosis) in order reduce the risk of myocardial infarction or thrombosis.

HIV infection and AIDS—C5a receptor modulators provided herein may be used to inhibit HIV infection, delay AIDS progression or decrease the severity of symptoms of HIV infection and AIDS.

In a further aspect, C5a receptor modulators may be used to perfuse a donor organ prior to transplantation of the organ into a recipient patient. Such perfusion is preferably carried out using a solution (e.g., pharmaceutical composition) comprising a concentration of the modulator that is sufficient to inhibit C5a receptor-mediated effects in vitro and/or in vivo. Such perfusion preferably reduces the severity or frequency of one or more of the inflammatory sequelae following organ transplantation when compared to that occurring in control (including, without restriction, historical control) transplant recipients who have received transplants of donor organs that have not been so perfused.

Within further aspects, C5a antagonists provided herein may be used to treat Alzheimer's disease, multiple sclerosis, and cognitive function decline associated with cardiopulmonary bypass surgery and related procedures. Such methods comprise administration of a therapeutically effective amount of a C5a antagonist provided herein to a patient afflicted with one or more of the above conditions, or who is considered to be at risk for the development of one or more such conditions.

Suitable patients include those patients suffering from or susceptible to a disorder or disease identified herein. Typical patients for treatment as described herein include mammals, particularly primates, especially humans. Other suitable patients include domesticated companion animals such as a dog, cat, horse, and the like, or a livestock animal such as cattle, pig, sheep and the like.

In general, treatment methods provided herein comprise administering to a patient a C5a receptor modulatory amount of one or more compounds provided herein. Treatment regimens may vary depending on the compound used and the particular condition to be treated; for treatment of most disorders, a frequency of administration of 4 times daily or less is preferred. In general, a dosage regimen of 2 times daily is more preferred, with once a day dosing particularly preferred. It will be understood, however, that the specific dose level and treatment regimen for any particular patient will depend upon a variety of factors including the activity of the specific compound employed, the age, body weight, general health, sex, diet, time of administration, route of administration, rate of excretion, drug combination (i.e., other drugs being administered to the patient) and the severity of the particular disease undergoing therapy, as well as the judgment of the prescribing medical practitioner. In general, the use of the minimum dose sufficient to provide effective therapy is preferred. Patients may generally be monitored for therapeutic effectiveness using medical or veterinary criteria suitable for the condition being treated or prevented.

As noted above, certain compounds and compositions provided herein are useful as inhibitors of C5a receptor-mediated chemotaxis (e.g., they may be used as standards in assays of such chemotaxis). Accordingly, methods are provided herein for inhibiting C5a receptor-mediated cellular chemotaxis, preferably leukocyte (e.g., neutrophil) chemotaxis. Such methods comprise contacting white blood cells (particularly primate white blood cells, especially human white blood cells) with one or more compounds provided herein. Preferably the concentration is sufficient to inhibit chemotaxis of white blood cells in an in vitro chemotaxis assay, so that the levels of chemotaxis observed in a control assay are significantly higher, as described above, than the levels observed in an assay to which a compound as described herein has been added.

Dosage levels of the order of from about 0.1 mg to about 140 mg per kilogram of body weight per day are useful in the treatment or prevention of conditions involving pathogenic C5a activity (about 0.5 mg to about 7 g per human patient per day). The amount of active ingredient that may be combined with the carrier materials to produce a single dosage form will vary depending upon the host treated and the particular mode of administration. Dosage unit forms will generally contain between from about 1 mg to about 500 mg of an active ingredient. For compounds administered orally, transdermally, intravaneously, or subcutaneously, it is preferred that sufficient amount of the compound be administered to achieve a serum concentration of 5 ng (nanograms)/mL-10 μg (micrograms)/mL serum, more preferably sufficient C5a receptor modulator to achieve a serum concentration of 20 ng-1 μg/mL serum should be administered, most preferably sufficient C5a receptor modulator to achieve a serum concentration of 50 ng/mL-200 ng/mL serum should be administered. For direct injection into the synovium (for the treatment of arthritis) sufficient C5a receptor modulator should be administered to achieve a local concentration of approximately 1 micromolar.

Frequency of dosage may also vary depending on the compound used and the particular disease treated. However, for treatment of most disorders, a dosage regimen of 4 times daily, three times daily, or less is preferred, with a dosage regimen of once daily or 2 times daily being particularly preferred. It will be understood, however, that the specific dose level for any particular patient will depend upon a variety of factors including the activity of the specific compound employed, the age, body weight, general health, sex, diet, time of administration, route of administration, and rate of excretion, drug combination (i.e., other drugs being administered to the patient), the severity of the particular disease undergoing therapy, and other factors, including the judgment of the prescribing medical practitioner.

Within separate aspects, the present invention provides a variety of non-pharmaceutical in vitro and in vivo uses for the compounds provided herein. For example, such compounds may be labeled and used as probes for the detection and localization of C5a receptor (in samples such as cell preparations or tissue sections, preparations or fractions thereof). Compounds may also be used as positive controls in assays for C5a receptor activity, as standards for determining the ability of a candidate agent to bind to C5a receptor, or as radiotracers for positron emission tomography (PET) imaging or for single photon emission computerized tomography (SPECT). Such methods can be used to characterize C5a receptors in living subjects. For example, a C5a receptor modulator may be labeled using any of a variety of well known techniques (e.g., radiolabeled with a radionuclide such as tritium, as described herein), and incubated with a sample for a suitable incubation time (e.g., determined by first assaying a time course of binding). Following incubation, unbound compound is removed (e.g., by washing), and bound compound detected using any method suitable for the label employed (e.g., autoradiography or scintillation counting for radiolabeled compounds; spectroscopic methods may be used to detect luminescent groups and fluorescent groups). As a control, a matched sample containing labeled compound and a greater (e.g., 10-fold greater) amount of unlabeled compound may be processed in the same manner. A greater amount of detectable label remaining in the test sample than in the control indicates the presence of C5a receptor in the sample. Detection assays, including receptor autoradiography (receptor mapping) of C5a receptor in cultured cells or tissue samples may be performed as described by Kuhar in sections 8.1.1 to 8.1.9 of Current Protocols in Pharmacology (1998) John Wiley & Sons, New York.

Modulators provided herein may also be used within a variety of well known cell separation methods. For example, modulators may be linked to the interior surface of a tissue culture plate or other support, for use as affinity ligands for immobilizing and thereby isolating, C5a receptors (e.g., isolating receptor-expressing cells) in vitro. Within one preferred embodiment, a modulator linked to a fluorescent marker, such as fluorescein, is contacted with the cells, which are then analyzed (or isolated) by fluorescence activated cell sorting (FACS).

Preparation of Compounds

Representative methods for preparing compounds of Formula I are shown in Schemes 1-2. Those skilled in the art will recognize that the reagents and synthetic transformations in the following Schemes can be readily modified to produce additional compounds of Formula I. When a protecting group is required, an optional deprotection step may be employed. Suitable protecting groups and methodology for protection and deprotection such as those described in Protecting Groups in Organic Synthesis by T. Greene are well known. Compounds and intermediates requiring protection/deprotection will be readily apparent.

The following abbreviations are used herein:

DMF dimethyl formamide

THF tetrahydrofuran

KOAc potassium acetate

18-crown-6 (1,4,7,10,13,16-hexaoxacyclooctadecane)

n-BuLi n-butyllithium

Pd(PPh₃)₄ tetrakis(triphenylphosphine) palladium (0)

NaBH₄ sodium borohydride

NBS N-bromosuccinimde

LC-MS liquid chromatography/mass spectrometry

¹H NMR proton nuclear magnetic resonance

MHz megahertz

Hz hertz

δ chemical shift

CDCl₃ deuterated chloroform

MS mass spectrometry

m/z mass/charge ratio

(M+1) mass+1 (also MH+)

eq. equivalents
Scheme 1. Preparation of compounds of Formula I where A is NR

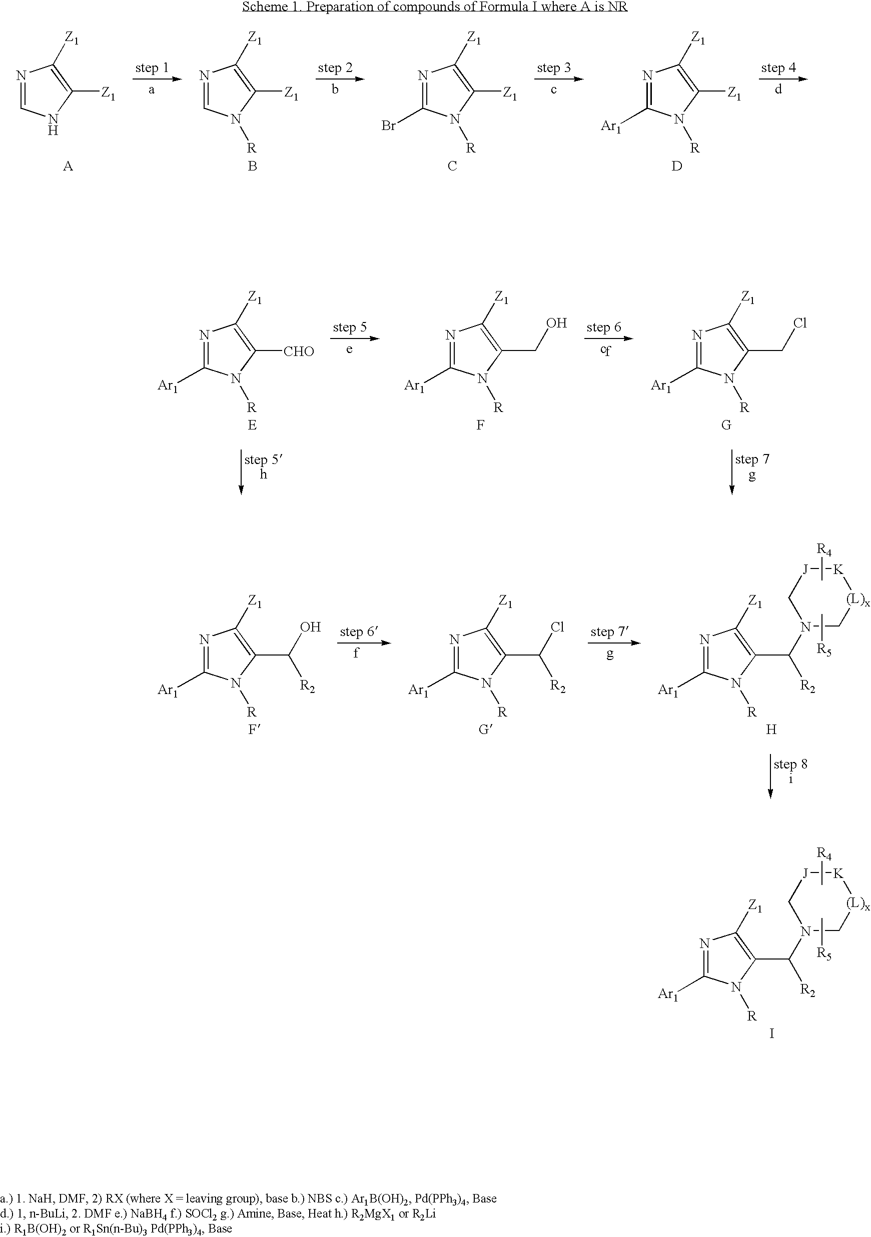

Scheme 1 illustrates a method for preparing compounds of Formula I wherein Z₁ is Cl or Br, A is N—R and Ar₁, R, R₁, R₂, R₄, R₅ and x are as defined in Formula I. In step 1, 4,5-dihaloimidazole A is treated with a suitable base such as sodium hydride in a suitable solvent such as DMF followed by an alkylating agent RX to obtain 1-alkyl-4,5-dihaloimidazole B. In step 1, X is a suitable leaving group such as bromo, iodo, mesylate, tosylate or triflate. Step 2 involves the electrophilic bromination of 1-alkyl-4,5-dihaloimidazole B with a suitable brominating agent such as NBS to obtain 1-alkyl-2-bromo-4,5-dihaloimidazole C. Where Z₁ is bromine, 2,4,5-tribromoimidazole may be alkylated as described in step 1 to directly obtain 1-alkyl-2,4,5-tribromoimidazole (C with Z₁═Br). In step 3,1-alkyl-2-bromo-4,5-dihaloimidazole C is coupled with a suitable metaloaryl derivative using transition metal catalysis (e.g. Suzuki reaction) to obtain 1-alkyl-2-aryl-4,5-dihaloimidazole D. Those skilled in the art will recognize that a broad array of metaloaryl derivatives (e.g., aryltin and arylzinc derivatives) and transition metal catalysts may be used in step 3 depending on the aryl group to be introduced. Step 4 involves transmetallation of halogen at the 5-position in 1-alkyl-2-aryl-4,5-dihaloimidazole D followed by reaction with DMF or a similar reagent to obtain aldehyde E. Aldehyde E may be reduced in step 5 to obtain alcohol F or reacted with a suitable organometallic reagent (e.g., R₂MgX₁, where X₁ is Cl, Br or I) in step 5′ to obtain alcohol F′. In step 6 and step 6′, alcohols F and F′ are converted to the corresponding chlorides G and G′. In step 7 and step 7′, chlorides G and G′ are reacted with suitable amines to obtain compounds of Formula I (H). Those skilled in the art will recognize that alternative leaving groups can be introduced in steps 6 and 6′ and subsequently employed in steps 7 and 7′. Further, alternative reaction conditions may be employed to convert aldehyde E to amino adduct H. For example, aldehyde E often may be converted to amino adduct H by reaction with appropriate amines under reductive amination conditions. In optional step 8, Z₁ is converted to a variety of substituents R₁. For example, a suitable organometallic reagent such as a boronic acid or organotin reagent may be coupled with H using transition metal catalysis (e.g., palladium (0)) to obtain compounds of Formula I (I). Similarly, alternative conditions may be employed in step 8 to prepare a wide variety of compounds of Formula I. For example, Z₁═Br can be replaced with a cyano substituents using transition metal catalysis. Those skilled in the art will further recognize that the sequence of reactions in Scheme 1 can optionally be modified to introduce R₁ reacting aldehyde E under the conditions described in step 8.

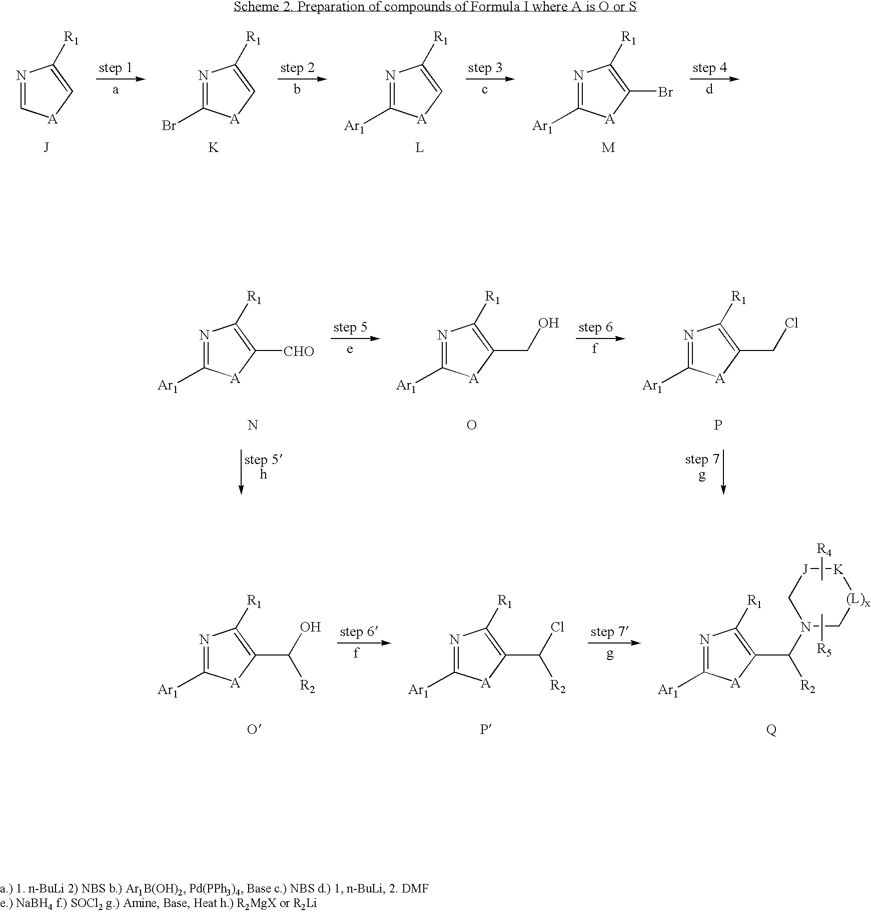

Scheme 2 illustrates a method for preparing compounds of Formula I wherein A is O or S and Ar₁, R₁, R₂, R₄, R₅ and x are as defined in Formula 1. In step 1,4-substituted oxazole or thiazole J is lithitated with a suitable base such as n-BuLi and reacted with a brominating agent such as NBS to obtain 2-bromo oxazole or thiazole K. Step 2 involves coupling of 2-bromo oxazole or thiazole K with a suitable metaloaryl derivative using transition metal catalysis (e.g., Suzuki reaction) to obtain 2-aryl oxazole or thiazole L. Those skilled in the art will recognize that a broad array of metaloaryl derivatives (e.g., aryltin and arylzinc derivatives) and transition metal catalysts may be used in step 2, depending on the aryl group to be introduced. In step 3, electrophilic bromination of 2-aryl oxazole or thiazole L with a suitable brominating agent such as NBS yields 5-bromo derivative M. Step 4 involves transmetallation of halogen at the 5-position in M followed by reaction with DMF or a similar reagent to obtain aldehyde N. Aldehyde N may be reduced in step 5 to obtain alcohol O or reacted with a suitable organometallic reagent (e.g., R₂MgX, where X is Cl, Br or I) in step 5′ to obtain alcohol O′. In step 6 and step 6′, alcohols O and O′ are converted to the corresponding chlorides P and P′. In step 7 and step 7′, chlorides P and P′ are reacted with suitable amines to obtain compounds of Formula I (Q). Those skilled in the art will recognize that alternative leaving groups can be introduced in steps 6 and 6′ and subsequently employed in steps 7 and 7′. Further, alternative reaction conditions may be employed to convert aldehyde N to amino adduct Q. For example, aldehyde N often may be converted to amino adduct Q by reaction with appropriate amines under reductive amination conditions.

Specific examples for the preparation of compounds of Formula I (and the other Formulas provided herein) by the methods illustrated in the above Schemes are provided in the following Examples. Unless otherwise specified all starting materials and reagents are of standard commercial grade, and are used without further purification, or are readily prepared from such materials by routine methods. Those skilled in the art of organic synthesis will recognize that starting materials and reaction conditions may be varied to achieve the desired end product.

### EXAMPLE 1

### Synthesis of 2-Benzo[1,3]Dioxol-5-Yl-1-[3-Butyl-5-Chloro-2-(2,6-Dimethyl-Phenyl)-3H-Imidazol-4-Ylmethyl]-Piperidine (10)

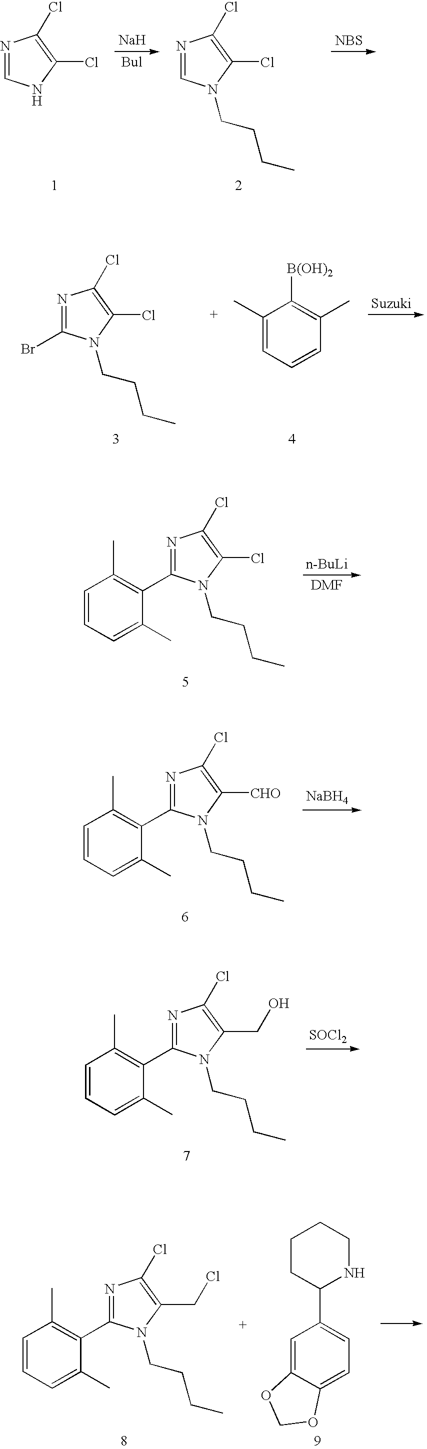

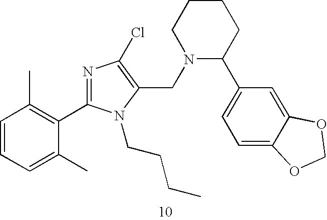

### Step A. 1-Butyl-4,5-dichloro-1H-imidazole (2).

Sodium hydride (1.05 mmol) is added to a solution of 3,4-dichloroimidazole (1, 1 mmol) in anhydrous DMF (5 mL) at 0° C. under nitrogen. After stirring at room temperature for 30 minutes, 1-iodobutane (1 mmol) is added and the reaction mixture is heated at 60° C. for 2 hours. The reaction mixture is cooled to room temperature, water is added, and the product is extracted with ethyl acetate. The organic layer is washed with water and brine and then dried over sodium sulfate. Evaporation and purification by chromatography on silica provides compound 2. LC-MS (M+1): 194.

### Step B. 2-Bromo-1-butyl-4,5-dichloro-1H-imidazole(3)

NBS (1.86 g, 1.05 mmol) is added to a solution of 1-butyl-4,5-dichloro-1H-imidazole (2, 1.95 g, 10 mmol) in acetonitrile (50 mL) at room temperature in portions. The reaction mixture is stirred at room temperature for 30 minutes. Ethyl acetate (100 mL) is added and the reaction mixture is washed with water, then brine, and dried over MgSO₄. The product is filtered and evaporated in vacuo to dryness. The crude product is purified by flash chromatography (hexane/ethyl acetate 100/5) to give compound 3. LC-MS (M+1): 271.

### Step C. 1-Butyl-4,5-dichloro-2-(2,6-dimethyl-phenyl)-1H-imidazole (5)

A solution containing 2-bromo-1-butyl-4,5-dichloro-1H-imidazole (3, 2.74 g, 10 mmol), 2,6-dimethylphenylboronic acid (4, 2.0 g, 12 mmol.) and Pd(PPh₃)₄ (0.23 mg, 0.2 mmol) in toluene/2M Na₂CO₃ (30 mL/15 mL) in a sealed tube is degassed, and then allowed to heat to 110° C. overnight. The organic layer is separated and concentrated in vacuo to dryness. The residue is purified by column chromatography on silica gel (hexane/ethyl acetate 100/5) to yield compound 5. LC-MS (M+1): 297.

### Step D. 3-Butyl-5-chloro-2-(2,6-dimethyl-phenyl)-3H-imidazole-4-carbaldehyde (6)

N-BuLi (1.6M in hexane, 9.4 mL, 15 mmol) is added dropwise at −78° C. to a solution of N-butyl-4,5-dichloro-2-(2,6-dimethyl-phenyl)-1H-imidazole (5, 3.27 g, 10 mmol.) in anhydrous THF. After addition, the reaction mixture is stirred at −78° C. for 2 hours. Anhydrous DMF (3 eq.) is then added in one portion. The mixture is stirred at −78° C. for 30 minutes, and then allowed to warm to room temperature slowly. The reaction mixture is stirred continuously overnight. The reaction is quenched with water and extracted with ethyl acetate, dried over MgSO₄, filtered, concentrated in vacuo, and purified via chromatography on silica gel to give compound 6. LC-MS (M+1): 291.

### Step E. [3-Butyl-5-chloro-2-(2,6-dimethyl-phenyl)-3H-imidazol-4-yl]-methanol (7)

Sodium borohydride (2 eq.) is added at room temperature to a solution of 3-butyl-5-chloro-2-(2,6-dimethyl-phenyl)-3H-imidazole-4-carbaldehyde (6, 291 mg, 1 mmol) in anhydrous methanol. The mixture was stirred for 2 hours. After removing the methanol, water is added and the product extracted with ethyl acetate. The organic phase is washed with brine, dried over anhydrous sodium sulfate, and concentrated. The residue is purified by column chromatography to yield compound 7. LC-MS (M+1): 293.

### Step F. 1-Butyl-4-chloro-5-chloromethyl-2-(2,6-dimethyl-phenyl)-1H-imidazole (8).

Thionyl chloride (0.2 mL) is added to a solution of [3-butyl-5-chloro-2-(2,6-dimethyl)-3H-imidazol-4-yl]-methanol (7, 30 mg, 0.1 mmol) in anhydrous chloroform (2 mL). The mixture is heated at 60° C. for 2 hours. The solvent and excess thionyl chloride are then removed under reduced pressure. The residue 8 is dried in vacuo and use for use in the next step without further purification.

### Step G. 2-Benzo[1,3]dioxol-5-yl-1-[3-butyl-5-chloro-2-(2,6-dimethyl-phenyl)-3H-imidazol-4-ylmethyl]-piperidine (10)

2-Benzo[1,3]dioxol-5-yl-piperidine (9, 21 mg, 0.1 mmol), potassium carbonate (28 mg, 0.2 mmol) and potassium iodide (10 mg) are added to a solution of 1-butyl-4-chloro-5-chloromethyl-2-(2,6-dimethyl-phenyl)-1H-imidazole (8, 0.1 mmol) in anhydrous acetonitrile (2 mL). The mixture is heated at 100° C. for 16 hours. After cooling to room temperature, the mixture is filtered though a short silica gel pad and the solids are washed with ethyl acetate. The filtrate is concentrated and the resulting residue purified by silica gel column chromatography to provide the title product 10 as a white solid. LC-MS (M+1): 481.

### EXAMPLE 2

### Preparation of 4-{1-[3-Butyl-5-Chloro-2-(2,6-Dimethyl-Phenyl)-3H-Imidazol-4-Ylmethyl]-Piperidin-2-Yl}-2-Hydroxy-Benzamide (19)

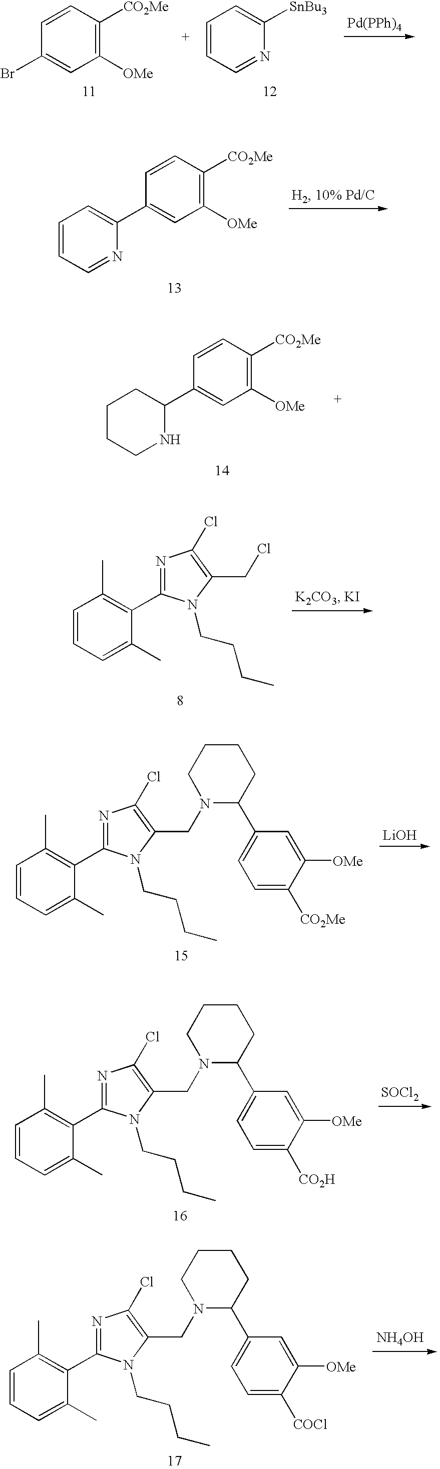

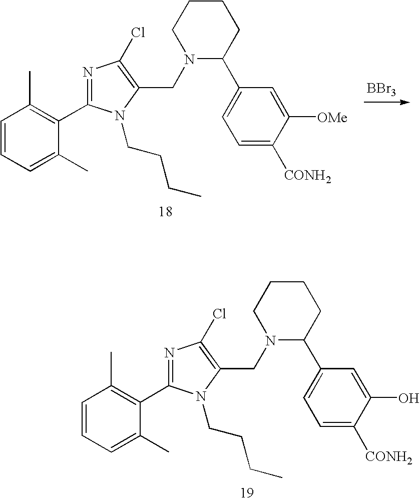

### Step A. 2-Methoxy-4-pyridin-2-yl-benzoic acid methyl ester (13)

2-Tributylstannanyl-pyridine (12, 7.6 g., 21 mmol) and tetrakis(triphenylphosphine) palladium(0) (300 mg) are added to a solution of 2-methoxy-4-piperidin-2-yl-benzoic acid methyl ester (11, 3 g, 14 mmol, prepared according to the reported procedure of Glennon et al. (1992) J. Med. Chent. 35(4):734-740) in anhydrous toluene (100 mL) under argon in a sealed tube. The mixture is heated at 100° C. for 16 hours. After cooling to room temperature, the mixture is poured into 2M sodium carbonate (100 mL) and the product is extracted with ethyl acetate. The extracts are dried over anhydrous sodium sulfate, concentrated, and purified by silica gel column chromatography to provide compound 13 as a light yellow oil. LC-MS (MH+): 244; ¹H NMR (400 MHz, CDCl₃) δ 8.71 (d, J=4.4 Hz, 1H), 7.90 (d, J=8.0 Hz, 1H), 7.77 (m, 1H), 7.75 (m, 1H), 7.52 (d, J=8.0 Hz, 1H), 7.30-7.25 (m, 1H), 4.02 (s, 3H), 3.91 (m, 3H).

### Step B. 2-Methoxy-4-piperidin-2-yl-benzoic acid methyl ester (14).

Hydrogen chloride ether solution (10 mL, 1 M) at 0° C. is slowly added to a solution of 2-methoxy-4-pyridin-2-yl-benzoic acid methyl ester (13, 1.2 g, 5 mmol) in ethyl acetate (20 mL). The solvents and excess of HCl are then removed under reduced pressure. The remaining salt is dissolved in methanol (40 mL) and hydrogenated with 10% palladium/carbon under pressure of 50 psi at room temperature for 16 hours. The catalyst is filtered through celite and the filtrate concentrated to provide compound 14 as a white solid. LC-MS (MH+): 250.

### Step C. 4-{1-[3-Butyl-5-chloro-2-(2,6-dimethyl-phenyl)-3H-imidazol-4-ylmethyl]-piperidin-2-yl}-2-methoxy-benzoic acid methyl ester (15)

This compound is prepared in the manner described in Example 1, step B from 1-butyl-4-chloro-5-chloromethyl-2-(2,6-dimethyl-phenyl)-1H-imidazole (8) and 2-methoxy-4-piperidin-2-yl-benzoic acid methyl ester (15). LC-MS (MH+): 496; ¹H NMR (400 MHz, CDCl₃) δ 7.76 (d, J=8.0 Hz, 1H), 7.21 (t, J=8.0 Hz, 1H), 7.09-7.04 (m, 3H), 6.99 (s, 1H), 3.92 (s, 3H), 3.87 (m, 3H), 3.76-3.69 (m, 2H), 3.45 (d, J=13.6 Hz, 1H), 3.42-3.36 (m, 1H), 3.04 (d, J=13.6 Hz, 1H), 3.05-3.01 (m, 1H), 2.90 (d, J=11.2 Hz, 1H), 2.09 (s, 3H), 2.0 (s, 3H), 1.83-1.59 (m, 4H), 1.54-1.31 (m, 3H), 1.23-1.12 (m, 1H), 1.09-0.96 (m, 2H), 0.67 (t, J=7.2 Hz, 3H).

### Step D. 4-{1-[3-Butyl-5-chloro-2-(2,6-dimethyl-phenyl)-3H-imidazol-4-ylmethyl]-piperidin-2-yl}-2-methoxy-benzoic acid (16)

Lithium hydroxide monohydrate (84 mg, 2 mmol) is added to a solution of 4-{1-[3-butyl-5-chloro-2-(2,6-dimethyl-phenyl)-3H-imidazol-4-ylmethyl]-piperidin-2-yl}-2-methoxy-benzoic acid methyl ester (15, 495 mg, 1 mmol) in mixed methanol-water-THF (3/1/1, 10 mL). The mixture is refluxed for 5 hours. After cooling to room temperature, the solution is neutralized to pH ˜6 using 1 N hydrochloric acid. This product is extracted with ethyl acetate and the extracts are washed with brine and dried over anhydrous sodium sulfate. Filtration and drying provide compound 16 as a white foam. LC-MS (MH+): 468; ¹H NMR (400 MHz, CDCl₃) δ 7.97 (d, J=6.4 Hz, 1H), 7.21 (t, J=7.6 Hz, 1H), 7.08-7.04 (m, 3H), 6.96 (br s, 1H), 3.92 (br s, 3H), 3.73-3.65 (m, 2H), 3.44 (d, J=13.6 Hz, 1H), 3.43-3.37 (m, 2H), 3.02 (d, J=13.6 Hz, 1H), 3.03 (m, 2H), 2.05 (s, 3H), 1.98 (s, 3H), 1.83-1.79 (m, 2H), 1.73-1.70 (m, 2H), 1.62-1.46 (m, 1H), 1.39-1.37 (m, 2H), 1.06-1.0 (m, 2H), 0.64 (t, J=7.2 Hz, 3H).

### Step E. 4-{1-[3-Butyl-5-chloro-2-(2,6-dimethyl-phenyl)-3H-imidazol-4-ylmethyl]-piperidin-2-yl}-2-methoxy-benzamide (18)

Thionyl chloride (0.4 mL) is added to a solution of 4-{1-[3-butyl-5-chloro-2-(2,6-dimethyl-phenyl)-3H-imidazol-4-ylmethyl]-piperidin-2-yl}-2-methoxy-benzoic acid (16, 102 mg, 0.2 mmol) in anhydrous benzene (5 mL). The mixture is refluxed for 1 hour. After removal of the solvent and excess of thionyl chloride, the residue 4-{1-[3-butyl-5-chloro-2-(2,6-dimethyl-phenyl)-3H-imidazol-4-ylmethyl]-piperidin-2-yl}-2-methoxy-benzoyl chloride (17) is dried in vacuo. A solution of the crude product 17 in anhydrous chloroform (5 mL) is added in one portion to a vigorously stirred ammonium hydroxide solution (30%, 5 mL) at room temperature. The mixture is stirred at room temperature overnight. The product is extracted with ethyl acetate and washed with brine. After removal of the solvent, the residue is purified by silica gel column chromatography to provide the desired product 18 as a white foam. LC-MS (MH+): 510; ¹H NMR (400 MHz, CDCl₃) δ 8.17 (d, J=8.0 Hz, 1H), 7.68 (m, 1H), 7.23-7.16 (m, 2H), 7.06 (m, 3H), 6.99 (s, 1H), 3.99 (s, 3H), 3.75-3.68 (m, 1H), 3.46 (d, J=13.6 Hz, 1H), 3.42-3.35 (m, 1H), 3.05 (d, J=13.6 Hz, 114), 2.91 (m, 1H), 2.05 (s, 3H), 1.99 (s, 3H), 1.83-1.65 (m, 4H), 1.57-1.32 (m, 4H), 1.27-1.13 (m, 2H), 1.08-0.99 (m, 2H), 0.67 (t, J=7.2 Hz, 3H).

### Step F. 4-{1-[3-Butyl-5-chloro-2-(2,6-dimethyl-phenyl)-3H-imidazol-4-ylmethyl]-piperidin-2-yl}-2-hydroxy-benzamide (19)

Tribromoborane (0.2 mL, 1 M in anhydrous dichloromethane) is added slowly to a solution of 4-{1-[3-butyl-5-chloro-2-(2,6-dimethyl-phenyl)-3H-imidazol-4-ylmethyl]-piperidin-2-yl}-2-methoxy-benzamide (18, 51 mg, 0.1 mmol) in anhydrous dichloromethane (2 mL) at −70° C. under argon. The mixture is then stirred for 16 hours. After cooling to 0° C., water is added slowly to quench the reaction and the product extracted with dichloromethane. The extracts are dried over anhydrous sodium sulfate, concentrated, and purified by silica gel column chromatography to provide compound 19 as a white solid. LC-MS (MH+): 496; ¹H NMR (400 MHz, CDCl₃) δ 12.18 (s, 1H), 7.33 (d, J=8.0 Hz, 1H), 7.22 (t, J=7.6 Hz, 1H), 7.09-7.05 (m, 3H), 6.87 (d, J=7.6 Hz, 1H), 3.85-3.77 (m, 1H), 3.48 (d, J=13.6 Hz, 1H), 3.42-3.35 (m, 1H), 3.04 (d, J=13.6 Hz, 1H), 3.0-2.98 (m, 1H), 2.90-2.87 (s, 1H), 2.05 (s, 3H), 2.01 (s, 3H), 1.80-1.61 (m, 4H), 1.53-1.30 (m, 3H), 1.25-1.14 (m, 2H), 1.01-0.98 (m, 2H), 0.69 (t, J=7.2 Hz, 3H).

### EXAMPLE 3

### Preparation of 3-{2-(2,6-Diethyl-Phenyl)-5-[2-(2,3-Dihydro-Benzo[1,4]Dioxin-6-Yl)-Piperidin-1-Ylmethyl]-1-Ethyl-1H-Imidazol-4-Yl}-Pyridine (30)

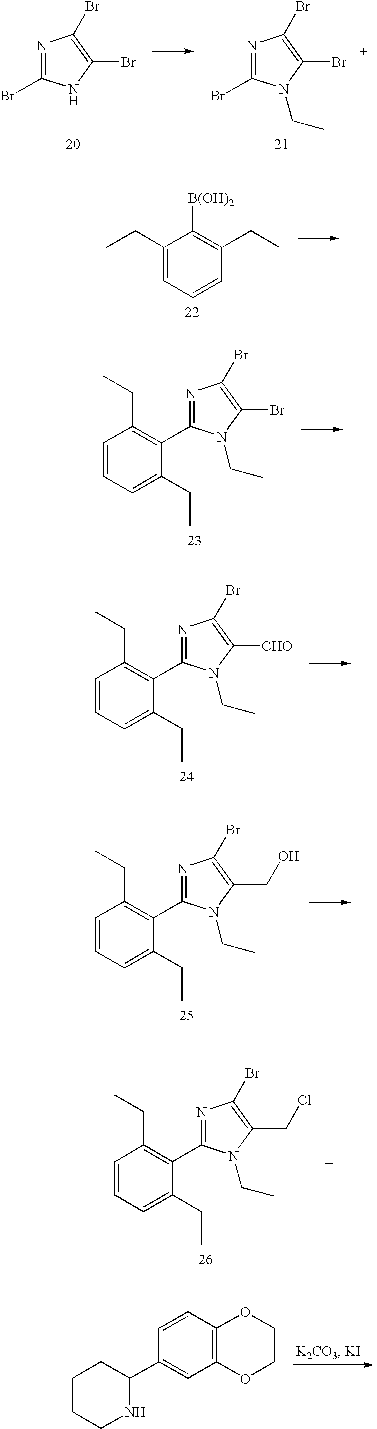

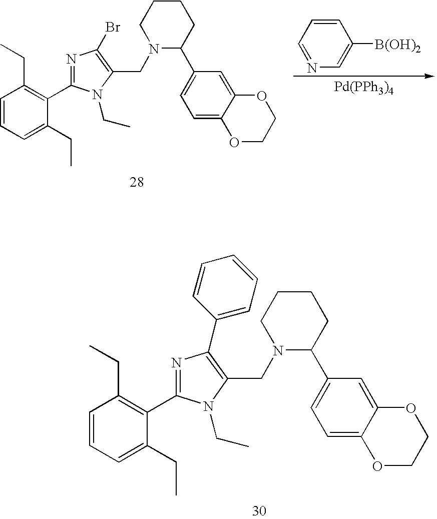

### Step A. 2,4,5-Tribromo-1-ethyl-1H-imidazole (21)

This compound is prepared as described in Example 1, step A from 2,4,5-tribromoimidazole (20). LC-MS (MH+): 332.

### Step B. 4,5-Dibromo-2-(2,6-diethyl-phenyl)-1-ethyl-1H-imidazole (23)

This compound is prepared as described in Example 1, step C from 2,4,5-Tribromo-1-ethyl-1H-imidazole (21) and 2,6-diethylphenylboronic acid (23, prepared according to the procedure described in U.S. patent application Ser. No. 10/405,989, filed Mar. 28, 2003, which is hereby incorporated by reference at pages 74-75 for its teachings regarding the synthesis of such compounds; the corresponding PCT application published as WO 03/082829 on Oct. 9, 2003). LC-MS (MH+): 386.

### Steps C, D, E and F. 1-[5-Bromo-2-(2,6-diethyl-phenyl)-3-ethyl-3H-imidazol-4-ylmethyl]-2-(2,3-dihydro-benzo[1,4]dioxin-6-yl)-piperidine (28)

This compound is prepared as described in Example 1, steps D, E, F and G, respectively, from compound 23. LC-MS (MH+): 540; ¹H NMR (400 MHz, CDCl₃) δ 7.33 (d, J=8.0 Hz, 1H), 7.12 (t, J=8.0 Hz, 2H), 6.89 (s, 1H), 6.85-6.78 (m, 2H), 4.22 (s, 4H), 3.97-3.89 (m, 1H), 3.53 (d, J=13.6 Hz, 1H), 3.47-3.39 (m, 1H), 2.98 (d, J=13.6 Hz, 1H), 2.93-2.88 (m, 2H), 2.30-2.20 (s, 4H), 2.04-1.98 (m, 1H), 1.76-1.57 (m, 4H), 1.53-1.33 (m, 2H), 1.16-1.06 (m, 6H), 0.96 (t, J=7.2 Hz, 3H).

### Step G. 3-{2-(2,6-Diethyl-phenyl)-5-[2-(2,3-dihydro-benzo[1,4]dioxin-6-yl)-piperidin-1-ylmethyl]-1-ethyl-1H-imidazol-4-yl}-pyridine (30)

3-Pyridyl boronic acid (29, 12 mg, 1 mmol) and tetrakis(triphenylphosphine)palladium(0) (5 mg) are added to a solution of 1-[5-bromo-2-(2,6-diethyl-phenyl)-3-ethyl-3H-imidazol-4-ylmethyl]-2-(2,3-dihydro-benzo[1,4]dioxin-6-yl)-piperidine (28, 32 mg 0.06 mmol) in mixed toluene (1 mL) and 2 M sodium carbonate (1 mL). The mixture is heated at 80° C. for 16 hours. After cooling to room temperature, the product is extracted with ethyl acetate. The extracts are washed with brine, dried over anhydrous sodium sulfate, and concentrated. The resulting residue is purified by silica gel column chromatography to provide the title product (30) as a white solid. LC-MS (MH+): 537.

### EXAMPLE 4

### Preparation of (R)-4-Benzyl-1-[5-Chloro-2-(2,6-Diethyl-Phenyl)-3-(2-Methoxy-Ethyl)-3H-Imidazol-4-Ylmethyl]-2-Phenyl-Piperazine (33)

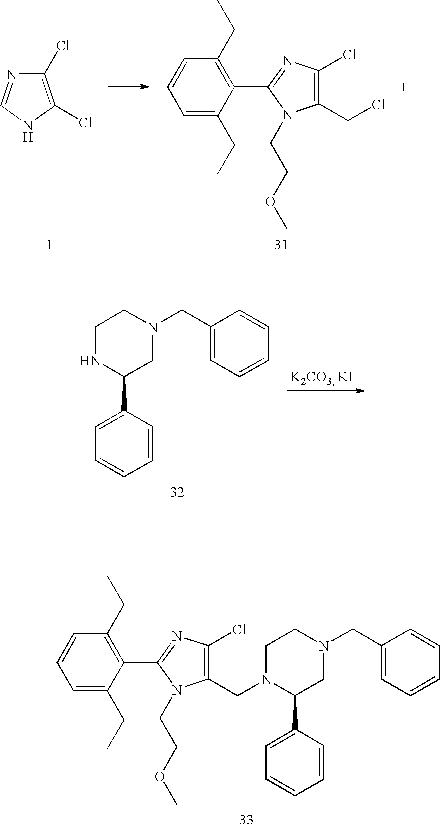

### Step A. 4-Chloro-5-chloromethyl-2-(2,6-diethyl-phenyl)-1-(2-methoxy-ethyl)-1H-imidazole (31)

This compound is prepared as described in Example 1, steps A, B, C, D, E and F from methoxyethyl bromide.

### Step B. 4-Benzyl-1-[5-chloro-2-(2,6-diethyl-phenyl)-3-(2-methoxy-ethyl)-3H-imidazol-4-ylmethyl]-2-phenyl-piperazine (33)

This compound is prepared as described in Example 1, step G from 4-chloro-5-chloromethyl-2-(2,6-diethyl-phenyl)-1-(2-methoxy-ethyl)-1H-imidazole (31) and (R)-1-benzyl-3-phenyl-piperazine (32). LC-MS (M⁺): 557; ¹H NMR (CDCl₃, δ ppm): 7.40 (2H, d, J=7.3 Hz), 7.2117.34 (9H, m), 7.12 (2H, dd, J=3.2, 7.7 Hz), 4.11 (1H, dt, J=7.0, 7.0 Hz), 3.56 (1H, d, J=13.7 Hz), 3.47-3.54 (3H, s & m), 3.33 (1H, dd, J=2.9, 10.3 Hz), 3.10-3.23 (3H, m), 3.00 (3H, s), 2.83-2.91 (2H, m), 2.77 (1H, dt, J=11.5, 2.5 Hz), 2.38 (1H, dt, J=2.5, 11.5 Hz), 2.14-2.31 (6H, m), 1.10 (6H, dt, J=11.5, 7.5 Hz).

### EXAMPLE 5

### Preparation of 2-Benzo[1,3]Dioxol-5-Yl-1-[2-(2,6-Diethyl-Phenyl)-4-Methyl-Thiazol-5-Ylmethyl]-Piperidine (43)

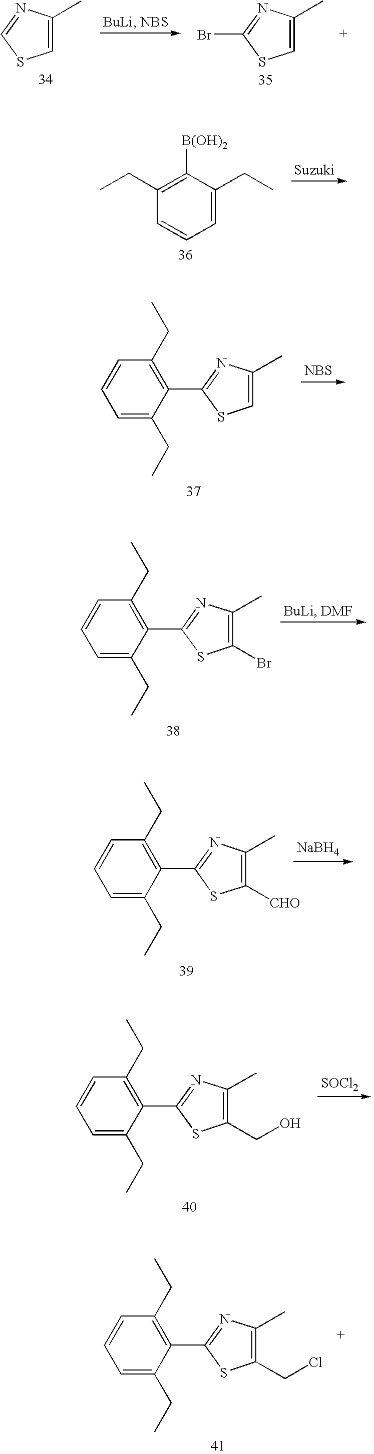

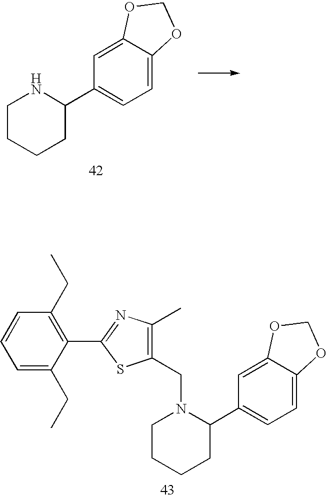

### Step A. 2-Bromo-4-methyl-thiazole (35).

A solution of n-butyl lithium in hexane (1.6M, 67 mL, 108 mmol, 1.05 eq.) is added dropwise to a solution of 4-methyl-thiazole 34 (10.17 g, 103 mmol) in 100 ml of anhydrous THF at −78° C. under nitrogen. After stirring the resulting mixture at −78° C. for 60 minutes, solid NBS (18.25 g, 103 mmol, 1.0 eq.) is added in portions. The resulting mixture is then stirred at −78° C. for 20 minutes, and warmed to room temperature over a 30 minute period. Saturated ammonium chloride (60 mL) is added to quench the reaction, the THF is evaporated, and the residue extracted with ether, washed with water and brine and dried over Na₂SO₄ Concentration and purification via silica gel chromatography (hexanes/ethyl acetate, from 8:1 to 5:1) affords compound 35. ¹HNMR (400 MHz, CDCl₃) δ 6.82 (1H, s), 2.43 (1H, s), MS (+1) m/z 178.

### Step B. 2-(2,6-Diethyl-phenyl)-4-methyl-thiazole (37)

Aqueous sodium carbonate (71 mL of 2.0 N) and 3.69 g of 2,6-diethylphenyl boronic acid (36, 20.7 mmol, 1.1 eq.) under nitrogen are added to a solution of 2-bromo-4-methyl-thiazole (35) (3.35 g, 18.8 mmol) and Pd(Ph₃)₄ (400 mg) in 100 mL of toluene. The resulting mixture is stirred at 120° C. for 24 hours. After cooling to room temperature, the reaction mixture is diluted with 100 mL of ethyl acetate, washed with water and brine, and dried over sodium sulfate. Concentration and purification via flash chromatography affords compound 37. ¹H NMR (400 MHz, CDCl₃) δ 7.32 (1H, t, J=7.6 Hz), 7.13 (2H, d, J=7.6 Hz), 7.00 (1H, s), 2.52 (3H, s), 2.43 (4H, q, J=7.6 Hz), 1.11 (6H, t, J=7.2 Hz); MS (M+1) m/z 232 (M⁺+1).

### Step C. 5-Bromo-2-(2,6-diethyl-phenyl)-4-methyl-thiazole (38)

NBS (644 mg, 3.6 mmol, 1.1 eq.) under nitrogen is added to a solution of 2-(2,6-diethyl-phenyl)-4-methyl-thiazole (37, 760 mg, 3.3 mmol) in 10 mL of acetonitrile. The resulting mixture is stirred at room temperature overnight. The reaction mixture is diluted with 30 mL of ethyl acetate, washed with water and brine, and dried over sodium sulfate. Concentration and purification via flash chromatography affords compound 38. MS (M+1) m/z 310.

### Step D. 2-(2,6-Diethyl-phenyl)-4-methyl-thiazole-5-carbaldehyde (39)

N-BuLi (1.6 M in hexane, 1.93 mL, 3.09 mmol, 1.2 eq.) is added to a solution of 5-bromo-2-(2,6-diethyl-phenyl)-4-methyl-thiazole (38, 800 mg, 2.58 mmol) in 20 mL of anhydrous THF cooled to −78° C. under nitrogen. The resulting solution is stirred at −78° C. for 60 minutes. The anion is quenched by the addition of 1 mL of anhydrous DMF. The reaction is warmed to room temperature slowly and then quenched by addition of water (20 mL). The THF is evaporated and the residue extracted with ethyl acetate, washed with water and brine, and dried over Na₂SO₄. Concentration and purification through silica gel chromatography (hexanes/ethyl acetate, from 8:1 to 5:1) affords compound 39. ¹H NMR (400 MHz, CDCl₃) δ 10.16 (1H, s), 7.37 (1H, t, J=7.6 Hz), 7.16 (2H, d, J=7.6 Hz), 2.83 (3H, s), 2.44(4H, q, J=7.2 Hz), 1.12 (6H, t, J=7.2 Hz). MS (+VE) m/z 260 (M⁺+1).

### Step E. [2-(2,6-Diethyl-phenyl)-4-methyl-thiazol-5-yl]-methanol (40)

NaBH₄ (50 mg) is added to a solution of 2-(2,6-diethyl-phenyl)-4-methyl-thiazole-5-carbaldehyde (39, 100 mg, 0.386 mmol) in 10 mL of methanol at 0° C. The resulting solution is stirred at 0° C. for 10 minutes. The methanol is then evaporated. The residue is diluted with 20 mL of ethyl acetate, washed with water and brine, and dried over Na₂SO₄. Concentration affords compound 40. MS (+VE) m/z 262 (M⁺+1).

### Step F. 2-Benzo[1,3]dioxol-5-yl-1-[2-(2,6-diethyl-phenyl)-4-methyl-thiazol-5-ylmethyl]-piperidine (43)

[2-(2,6-Diethyl-phenyl)-4-methyl-thiazol-5-yl]-methanol (40, 35 mg, 0.133 mmol) is dissolved in 5 mL of dichloromethane and cooled to 0° C. Thionyl chloride (5 eq.) is added to this solution. The resulting solution is stirred at room temperature for 2 hours. The solvent and excess of thionyl chloride are evaporated. Toluene (5 ml) is added and the solvent is again evaporated from the residue again to remove the remaining thionyl chloride. The residue 41 is dissolved in 2 mL of anhydrous acetonitrile. This solution is added to an ice-cooled solution of 2-piperonyl piperidine (42, 33 mg, 0.16 mmol, 1.2 eq.) in 3 mL of acetonitrile containing 2 eq. of potassium carbonate. The resulting mixture is stirred at 80° C. for 8 hours, then diluted with 20 mL of ethyl acetate, washed with water and brine, dried and concentrated. The residue is purified by silica gel chromatography (hexanes/ethyl acetate, from 10:1 to 8:1) to give compound 43. ¹H NMR (400 MHz, CDCl₃) δ 7.30 (1H, t, J=8.0 Hz), 7.11 (2H, d, J=7.6 Hz), 6.97 (1H, s), 6.84 (1H, J=8.0 Hz), 6.75 (1H, d, J=8.0 Hz), 5.93 (2H, d, J=3.6 Hz), 3.79 (1H, d, J=14.4 Hz), 3.20 (1H, d, J=14.4 Hz), 3.10 (2H, m), 2.44 (4H, q, J=8.0 Hz), 2.31 (3H, s), 2.07 (1H, m), 1.80-1.24 (6H, m), 1.12 (6H, t, J=7.6 Hz); MS (+VE) m/z 449 (M⁺+1).

### EXAMPLE 6

### Additional (Heterocycloalkyl)Methyl Imidazole Derivatives

### I. Preparation of 4-{1-Butyl-4-chloro-5-[2-(4-methoxy-phenyl)-piperidin-1-ylmethyl]-1H-imidazol-2-yl}-5-methyl-1H-indazole (49)

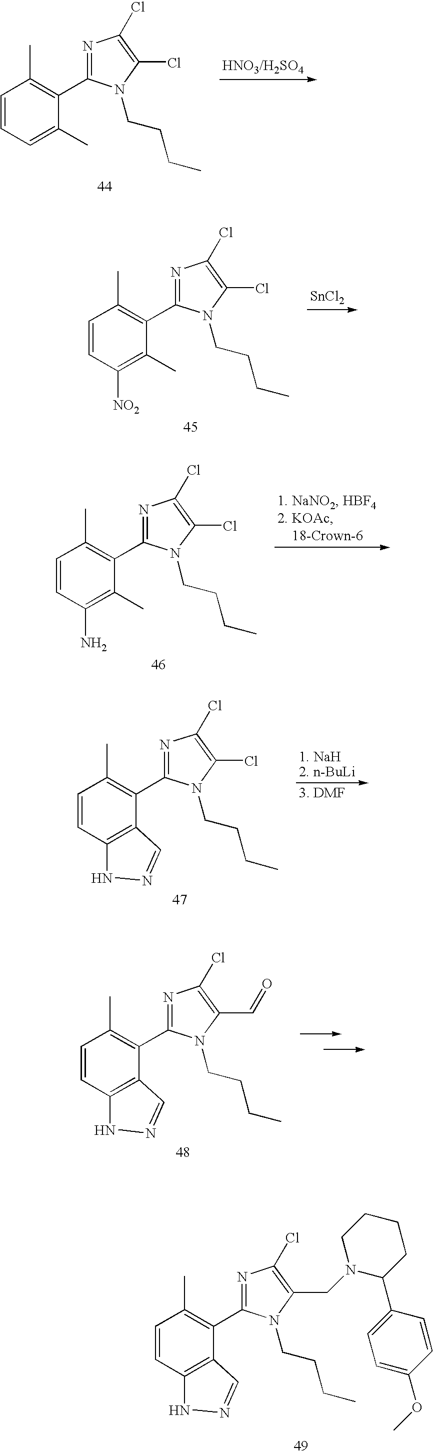

### Step A. 1-Butyl-4,5-dichloro-2-(2,6-dimethyl-3-nitro-phenyl)-1H-imidazole (45)

1-Butyl-4,5-dichloro-2-(2,6-dimethyl-phenyl)-1H-imidazole 44 (2.0 g) is treated with a 1:1 mixture of concentrated sulfuric acid (10 ml) and fuming nitric acid (10 ml) at −10° C. to 0° C. for 1 hour. The reaction mixture is poured into ice, neutralized with 6 N NaOH to pH 7-8 and extracted with ethyl acetate (50 ml×2). The ethyl acetate extract is dried over sodium sulfate and evaporated at reduced pressure. Crude 1-butyl-4,5-dichloro-2-(2,6-dimethyl-3-nitro-phenyl)-1H-imidazole 45 is used without further purification.

### Step B. 3-(1-butyl-4,5-dichloro-1H-imidazol-2-yl)-2,4-dimethyl-phenylamine (46)

1-Butyl-4,5-dichloro-2-(2,6-dimethyl-3-nitro-phenyl)-1H-imidazole 45 (1.9 g) is added to cooled concentrated hydrochloric acid (10 ml) followed by portionwise addition of excess tin (II) chloride (2.0 g) at 0° C. After stirring at 0° C. for 2 hours, the reaction mixture is poured into ice and neutralized with 10 N NaOH to pH 7-8, extracted with Et₂O twice, dried over MgSO₄, and purified by chromatography on silica gel to obtain 3-(1-butyl-4,5-dichloro-1H-imidazol-2-yl)-2,4-dimethyl-phenylamine 46.

### Step C. 4-(1-Butyl-4,5-dichloro-1H-imidazol-2-yl)-5-methyl-1H-indazole (47)

To a suspension of 3-(1-butyl-4,5-dichloro-1H-imidazol-2-yl)-2,4-dimethyl-phenylamine 46 (1.3 g) in HBF₄ (48%) (10 ml) is added dropwise a solution of NaNO₂ (1.15 eq., 0.33 g) in water (10 ml) at 0° C. over 10 minutes. The reaction mixture is allowed to slowly warm to room temperature over 2 hours and the suspended solid is collected by filtration, washed with cooled water and dried under vacuum overnight. The resulting residue (1.1 g) is dissolved in chloroform (10 ml), treated with potassium acetate (2.0 eq., 0.54 g) and 18-crown-6 (0.1 eq., 19 mg) and stirred at room temperature for 4 hours. The reaction mixture is washed with water, dried over MgSO₄, and purified by chromatography on silica gel (hexanes/ethyl acetate 2:1) to yield 4-(1-butyl-4,5-dichloro-1H-imidazol-2-yl)-5-methyl-1H-indazole 47.

### Step D. 3-Butyl-5-chloro-2-(5-methyl-H-indazol-4-yl)-3H-imidazole-4-carbaldehyde (48)

A solution of 4-(1-butyl-4,5-dichloro-1H-imidazol-2-yl)-5-methyl-1H-indazole 47 (0.24 g) in THF (5 ml) with stirring is treated with NaH (60%) (33 mg) at 0° C. for 1 hour, cooled to −78° C. and treated with 1 eq. of t-BuLi (1.7 M in hexanes, 0.5 ml) dropwise. After 1 hour, 1 eq. of DMF (0.5 ml) is added and the reaction mixture is allowed to warm to room temperature over 1 hour. The reaction is quenched with water, extracted with ethyl acetate, dried over MgSO₄, filtered and evaporated. The resulting residue is purified by chromatography on silica gel (hexanes/ethyl acetate 10:1 to 2:1) to give 3-butyl-5-chloro-2-(5-methyl-1H-indazol-4-yl)-3H-imidazole-4-carbaldehyde 48.

### Step E. 4-{1-Butyl-4-chloro-5-[2-(4-methoxy-phenyl)-piperidin-1-ylmethyl]-1H-imidazol-2-yl}-5-methyl-1H-indazole (49)

4-{1-Butyl-4-chloro-5-[2-(4-methoxy-phenyl)-piperidin-1-ylmethyl]-1H-imidazol-2-yl}-5-methyl-1H-indazole is prepared by alkylation of 3-butyl-5-chloro-2-(5-methyl-1H-indazol-4-yl)-3H-imidazole-4-carbaldehyde 48 with 2-(4-methoxy-phenyl)-piperidine following the procedure given in Steps E, F and G in Example 1.

### II. Preparation of 2-Benzo[1,3]dioxol-5-yl-1-[2-(2,6-diethyl-phenyl)-4-phenyl-oxazol-5-ylmethyl]-piperidine (56)

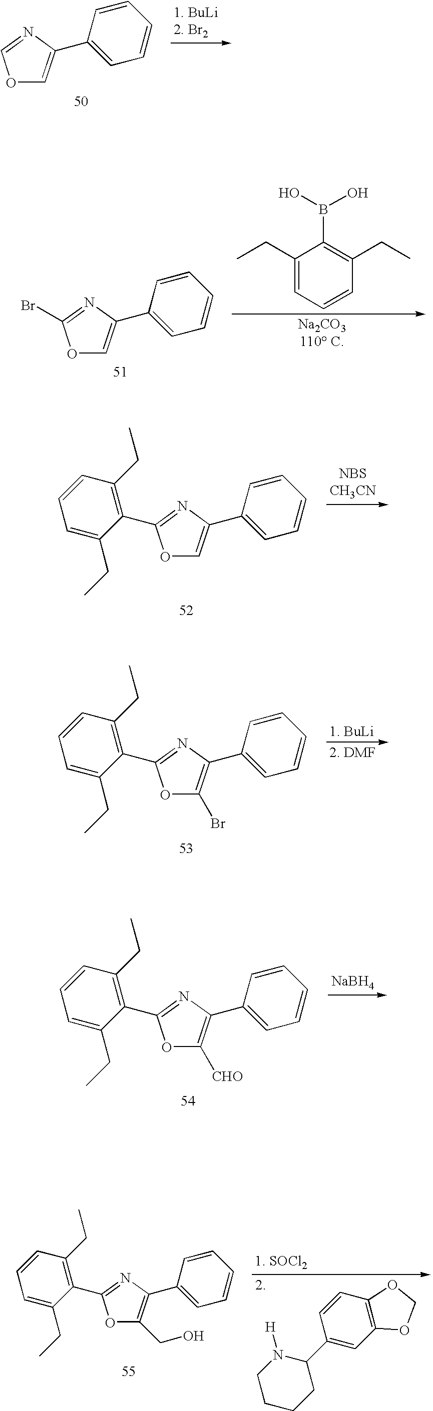

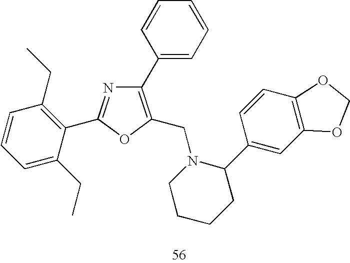

### Step A. 2-Bromo-4-phenyl-oxazole (51)

To a solution of 4-phenyl-oxazole 50 (1.49 g, 10.3 mmol) in 50 mL of anhydrous THF at −78° C. under nitrogen, a solution of butyl lithium in hexane (1.6 M, 8 mL, 12.8 mmol, 1.3 eq.) is added dropwise. The resulting mixture is stirred at −78° C. for 60 min, then anhydrous bromine (1.91 g, 11.9 mmol, 1.2 eq.) is added in portions. The resulting mixture is stirred at −78° C. for 20 minutes, and then raised to room temperature slowly. Saturated ammonium chloride solution (30 mL) is added, THF is evaporated at reduced pressure and the resulting residue is extracted with ether, washed with water and brine, and dried over Na₂SO₄ Concentration and purification by silica gel chromatography (hexanes/ethyl acetate, from 8:1 to 5:1) affords 2-bromo-4-phenyl-oxazole 51 as low melting point solid. ¹H NMR (400 MHz, CDCl₃) δ 7.92 (1H, s), 7.70(2H, d, J=8.0 Hz), 7.32-7.45 (3, m), MS (+VE) m/z 225 (M⁺).

### Step B. 2-(2,6-Diethyl-phenyl)-4-phenyl-oxazole (52)

To a solution of compound 2-bromo-4-phenyl-oxazole 51 (780 mg, 3.48 mmol) and Pd[(Ph)₃]₄ (100 mg) in 50 mL of toluene is added 5 mL of 2.0 N aqueous sodium carbonate and 683 mg of 2,6-diethylphenylboronic acid (3.83 mmol, 1.1 eq.) under nitrogen. The resulting mixture is stirred at 110° C. for 24 hours. After being cooled to room temperature, the reaction mixture is diluted with 50 mL of ethyl acetate, washed with water and brine, dried over sodium sulfate. Concentration and purification through flash chromatography affords 2-(2,6-diethyl-phenyl)-4-phenyl-oxazole 52. ¹H NMR (400 MHz, CDCl₃) δ 8.06 (1H, s), 7.84 (2H, dd, J=1.6, 8.4 Hz), 7.31˜7.46 (4H, m), 7.17 (2H, d, J=7.8 Hz), 2.55 (2H, q, J=7.8 Hz), 1.17 (3H, t, J=7.8 Hz). MS (+VE) m/z 278 (M⁺+1).

### Step C. 5-Bromo-2-(2,6-diethyl-phenyl)-4-phenyl-oxazole (53)

To a solution of 2-(2,6-diethyl-phenyl)-4-phenyl-oxazole 52 (670, 2.41 mmol) in 10 mL of acetonitrile is added NBS (473 mg, 2.65 mmol, 1.1 eq.) under nitrogen and the resulting mixture is stirred at room temperature for 18 hours. The reaction mixture is diluted with 30 mL of ethyl acetate, washed with water and brine, dried over sodium sulfate. Concentration and purification through flash chromatography affords 5-bromo-2-(2,6-diethyl-phenyl)-4-phenyl-oxazole 53 MS (+VE) m/z 356 (M⁺).

### Step D. 2-(2,6-Diethyl-phenyl)-4-phenyl-oxazole-5-carbaldehyde (54)

To a solution 5-bromo-2-(2,6-diethyl-phenyl)-4-phenyl-oxazole 53 (662 mg, 1.86 mmol) in 20 mL anhydrous tetrahydrofuran cooled to −78° C. is added n-BuLi (1.6 M in hexane, 1.39 ml, 2.23 mmol, 1.2 eq.) under nitrogen. The resulting solution is stirred at −78° C. for 60 minutes, then the anion is quenched by addition of 1 mL of anhydrous DMF. The reaction mixture is warmed to room temperature slowly, and stirred at room temperature for 10 minutes. 20 mL of water is added to quench the reaction, THF is evaporated at reduced pressure, and the resulting residue is extracted with ethyl acetate, washed with water and brine and dried over Na₂SO₄. Concentration and purification by silica gel chromatography (hexanes/ethyl acetate, from 8:1 to 5:1) affords 2-(2,6-diethyl-phenyl)-4-phenyl-oxazole-5-carbaldehyde 54. MS (+VE) m/z 306 (M⁺+1).

### Step E. [2-(2,6-Diethyl-phenyl)-4-phenyl-oxazol-5-yl]-methanol (55)

To a solution of 2-(2,6-diethyl-phenyl)-4-phenyl-oxazole-5-carbaldehyde 54 (306 mg, 1.0 mmol) in 10 mL methanol cooled to 0° C. is added NaBH₄ (100 mg). The resulting solution is stirred at 0° C. for 30 minutes, then the methanol is evaporated and the residue is diluted with 20 mL of ethyl acetate, washed with water and brine and dried over Na₂SO₄. Concentration affords [2-(2,6-diethyl-phenyl)-4-phenyl-oxazol-5-yl]-methanol 55. MS (+VE) m/z 308 (M⁺+1).

### Step F. 2-Benzo[1,3]dioxol-5-yl-1-[2-(2,6-diethyl-phenyl)-4-phenyl-oxazol-5-ylmethyl]-piperidine (56)

[2-(2,6-Diethyl-phenyl)-4-phenyl-oxazol-5-yl]-methanol 55 (31 mg, 0.1 mmol) is dissolved in 5 mL of dichloromethane and cooled to 0° C., and 5 eq. of thionyl chloride is added. The resulting solution is stirred at room temperature for 2 hours. Excess thionyl chloride is evaporated, and 5 ml of toluene is added to the residue and evaporated again to remove any remaining thionyl chloride. The residual crude product is dissolved in 2 mL of anhydrous acetonitrile and added to an ice-cooled solution of 2-benzo[1,3]dioxol-5-yl-piperidine (25 mg, 0.12 mmol, 1.2 eq.) in 3 mL of acetonitrile containing 2 eq. of potassium carbonate. The resulting mixture is stirred at 80° C. for 8 hours, diluted with 20 mL of ethyl acetate, washed with water and brine, dried and concentrated, and the residue is purified through silica gel chromatography (hexanes/ethyl acetate, from 10:1 to 8:1) to give 2-benzo[1,3]dioxol-5-yl-1-[2-(2,6-diethyl-phenyl)-4-phenyl-oxazol-5-ylmethyl]-piperidine 56. MS (+VE) m/z 495 (M⁺+1).

### III. Representative Medium Speed Synthesis Protocols

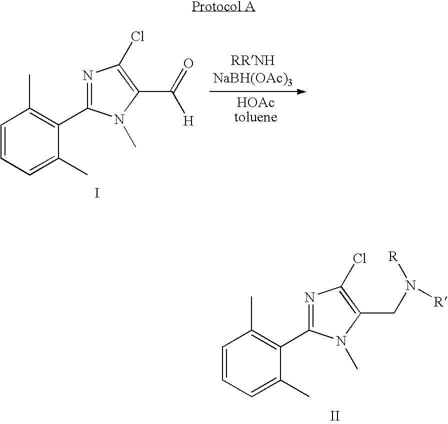

To aldehyde I (0.15 mL of a 0.2M solution in toluene) is added a secondary amine (0.1 mL of a 0.2M solution in toluene) followed by NaBH(OAc)₃ (0.5 mL of a 0.2M suspension in benzene), and then HOAc (0.2 mL of a 0.2M solution in toluene). The reaction mixture is sealed then incubated at 50° C. for 16 hours. The reaction is cooled to room temperature, and then quenched by the addition of 0.5 mL saturated NaHCO₃ solution. The organic phase is transferred to a 500 mg SCX cartridge (UCT CUBCX156). The cartridge is washed with 4 mL EtOAc to remove impurities, and then eluted with 10:1:1 EtOAc:MeOH:Triethylamine to collect products. The solvent is evaporated to afford the pure product II.

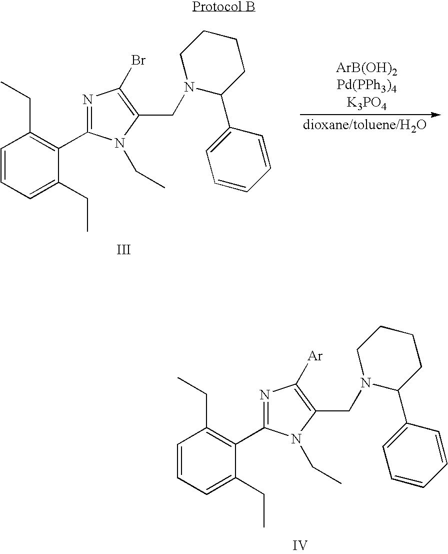

To imidazobromide III (0.1 mL of a 0.2M solution in dioxane) is added an arylboronic acid (0.2 mL of a 0.2M solution in dioxane) followed by K₃PO₄ (0.05 mL of a 1M solution in H₂O). The reaction mixture is placed in a glove box under N₂ atmosphere, and then Pd(PPh₃)₄ (0.1 mL of a 0.01M solution in toluene) is added. The reaction mixture is sealed and then incubated at 80° C. for 16 hours. The reaction mixture is cooled to room temperature. Sodium hydroxide (0.5 mL of a 1M solution in water) is added and the reaction mixture extracted with 0.5 mL ethyl acetate. The organic phase is transferred to a 500 mg SCX cartridge (UCT CUBCX156) and eluted with 4 mL ethyl acetate to remove impurities followed by 4 mL 10:1:1 EtOAc:MeOH:triethylamine to elute the product. The solvent is removed to afford pure product IV.

### IV. Additional Compounds

The compounds shown in Tables I and II, below, are synthesized via methods described above. In certain circumstances starting materials or reactions conditions may be modified. Such modification would be readily understood by those of ordinary skill in the art of organic chemical synthesis. For example, diastereomers are prepared by the methods given above, using enantionmerically impure starting material in the amine coupling step to provide these compounds as a mixture; the diastereomers are then separated by column chromatography. Compounds in Table II were prepared according to protocol A or protocol B, above, as indicated.

Compounds shown in Tables I and II exhibit a Kᵢ of less than 2 μM in the standard assay of C5a receptor mediated calcium mobilization given in Example 17.

The LC/MS data presented in Tables I and II were obtained using the following instrumentation and methods. MS spectroscopy data is Electrospray MS, obtained in positive ion mode, with a 15V Cone voltage, using a WATERS ZMD 2000 Mass Spec Detector, equipped with a WATERS 600 pump, WATERS 2487 Dual Wavelength Detector, GILSON 215 Autosampler, and a GILSON 841 Microinjector. MassLynx version 3.4 software was used for data collection and analysis.

Sample, 2-20 μL, was injected onto a 33×4.6 mm YMC ProPack C18; 5 u column, and eluted using a 2-phase linear gradient at a 4 mL/minute flow rate. Sample was detected at 220 and 254 nm. The elution conditions were as follows: Mobile Phase A—95/5/0.1 Water/Methanol/TFA, Mobile Phase B—5/95/0.1 Water/Methanol/TFA.

Gradient-

| time(min) | % B |
|---|---|
| 0 | 10 |
| 0.01 | 10 |
| 2.0 | 100 |
| 3.5 | 100 |
| 3.51 | 10 |
| 3.52 | |

The total run time for the gradient was 4.0 minutes.

TABLE 1

| | | | Ret. | LCMS | LCMS |
|---|---|---|---|---|---|
| | Compound | Name | Time | Mass | M+H |
| 57 | 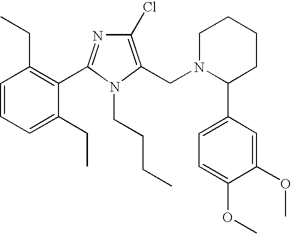 | 1-{[1-butyl-4-chloro-2- (2,6-diethylphenyl)-1H- imidazol-5-yl]methyl}-2- (3,4-dimethoxyphenyl) piperidine | 1.15 | 523.3 | 524.3 |
| 58 | 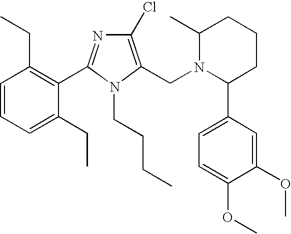 | 1-{[1-butyl-4-chloro-2-(2,6-diethylphenyl)-1H- imidazol-5-yl]methyl}-2- (3,4-dimethoxyphenyl)- 6-methylpiperidine | 1.16 | 537.3 | 538.4 |
| 59 | 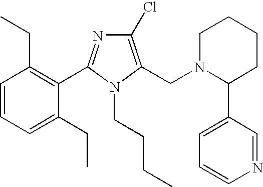 | 3-(1 -{[1-butyl-4-chloro- 2-(2,6-diethylphenyl)- 1H-imidazol-5- yl]methyl}piperidin-2- yl)pyridine | 1.18 464.3 | 465.3 | |
| 60 | 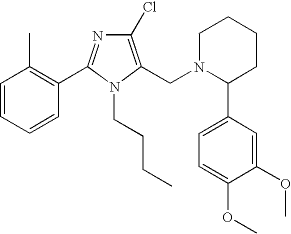 | 1-{[1 -butyl-4-chloro-2- (2-methylphenyl)-1H- imidazol-5-yl]methyl}-2- (3,4-dimethoxyphenyl) piperidine | 1.1 | 481.2 | 482.3 |
| 61 | 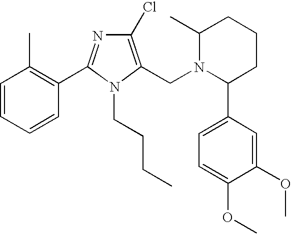 | 1-{[1-butyl-4-chloro-2- (2-methylphenyl)-1H- imidazol-5-yl]methyl}-2- (3,4-dimethoxyphenyl)- 6-methylpiperidine | 1.1 | 495.3 | 496.3 |
| 62 | 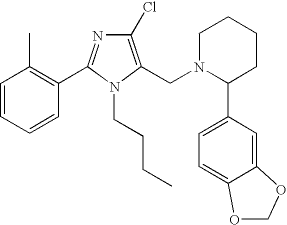 | 2-(1,3-benzodioxol-5-yl)- 1-{[1-butyl-4-chloro-2- (2-methylphenyl)-1H- imidazol-5- yl]methyl}piperidine | 1.12 | 465.2 | 466.2 |
| 63 | 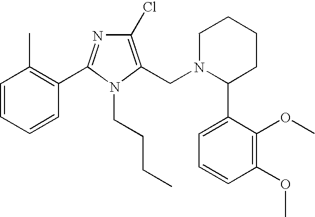 | 1-{[1-butyl-4-chloro-2- (2-methylphenyl)-1H- imidazol-5-yl]methyl}-2- (2,3-dimethoxyphenyl) piperidine | 1.13 | 481.2 | 482.3 |
| 64 | 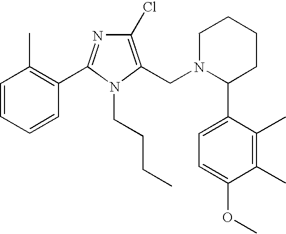 | 1-{[1-butyl-4-chloro-2- (2-methylphenyl)-1H- imidazol-5-yl]methyl}-2- (4-methoxy-2,3- dimethylphenyl) piperidine | 1.17 | 479.3 | 480.3 |
| 65 | 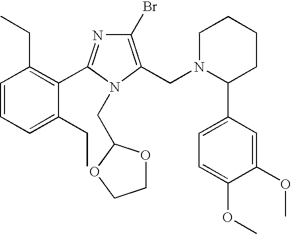 | 1-{[4-bromo-2-(2,6- diethylphenyl)-1-(1,3- dioxolan-2-yl]methyl)- 1H-imidazol-5- 1]methyl}-2-(3,4- dimethoxyphenyl) piperidine | 1.09 | 597.2 | 598.5 |
| 66 | 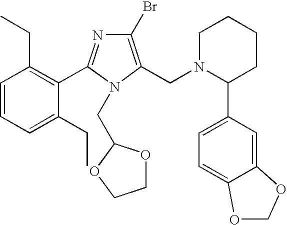 | 2-(1,3-benzodioxol-5-yl)- 1-{[4-bromo-2-(2,6- diethylphenyl)-1-{1,3- dioxolan-2-ylmethyl)- 1H-imidazol-5- yl]methyl}piperidine | 1.15 | 581.2 | 582.1 |
| 67 | 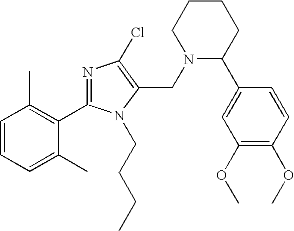 | 1-{[1-butyl-4-chloro-2- (2,6-dimethylphenyl)- 1H-imidazol-5- yl]methyl}-2-(3,4- dimethoxyphenyl) piperidine | 1.15 | 495.3 | 496.3 |
| 68 | 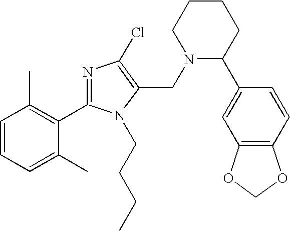 | 2-(1,3-benzodioxol-5-yl)- 1-{[1-butyl-4-chloro-2- (2,6-dimethylphenyl)- 1H-imidazol-5- yl]methyl}piperidine | 1.17 | 479.2 | 480.2 |
| 69 | 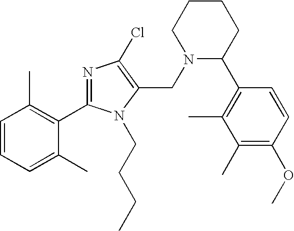 | 1-{[1-butyl-4-chloro-2- (2,6-dimethylphenyl)- 1H-imidazol-5- yl]methyl}-2-(4- methoxy-2,3- dimethylphenyl) piperidine | 1.22 | 493.3 | 494.3 |
| 70 | 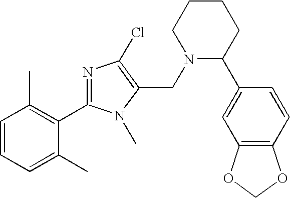 | 2-(1,3-benzodioxol-5-yl)- 1-{[4-chloro-2-(2,6- dimethylphenyl)-1- methyl-1H-imidazol-5- yl]methyl}piperidine | 1.11 | 437.2 | 438.2 |
| 71 | 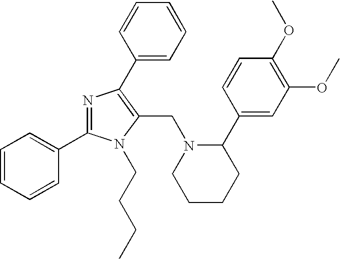 | 1-[(1-butyl-2,4-diphenyl- 1H-imidazol-5- yl)methyl]-2-(3,4- dimethoxyphenyl)piperidine | 1.13 | 509.3 | 510.3 |
| 72 | 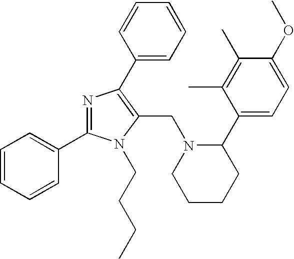 | 1-[(1-butyl-2,4-diphenyl- 1H-imidazol-5- yl)methyl]-2-(4- methoxy-2,3- dimethylphenyl) piperidine | 1.19 | 507.3 | 508.3 |
| 73 | 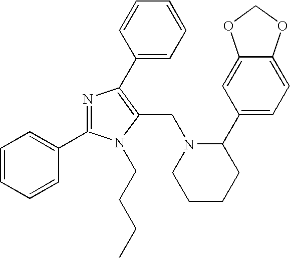 | 2-(1,3-benzodioxol-5-yl)- 1-[(1-butyl-2,4-diphenyl- 1H-imidazol-5- yl)methyl]piperidine | 1.17 | 493.3 | 494.3 |
| 74 | 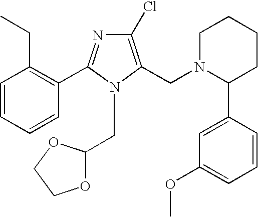 | 1-{[4-chloro-1-(1,3- dioxolan-2-ylmethyl)-2- (2-ethylphenyl)-1H- imidazol-5-yl]methyl}-2- (3- methoxyphenyl)piperidine | 1.08 | 495.2 | 496.4 |
| 75 | 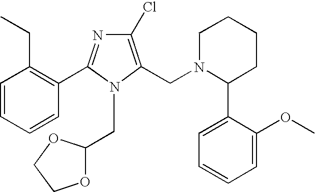 | 1-{[4-chloro-1-(1,3- dioxolan-2-ylmethyl)-2- (2-ethylphenyl)-1H- imidazol-5-yl]methyl}-2- (2-methoxyphenyl) piperidine | 1.07 | 495.2 | 496.4 |
| 76 | 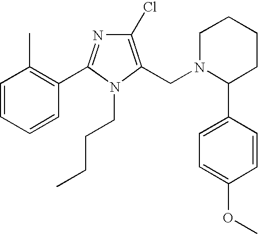 | 1-{[1-butyl-4-chloro-2- (2-methylphenyl-1H- imidazol-5-yl]methyl)-2- (4-methoxyphenyl) piperidine | 1.12 | 451.2 | 452.4 |
| 77 | 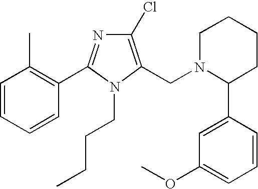 | 1-{[1-butyl-4-chloro-2- (2-methylphenyl)-1H- imidazol-5-yl]methyl}-2- (3-methoxyphenyl) piperidine | 1.13 | 451.2 | 452.4 |
| 78 | 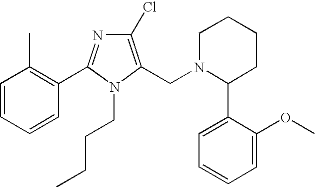 | 1-{[1-butyl-4-chloro-2- (2-methylphenyl)-1H- imidazol-5-yl]methyl}-2- (2-methoxyphenyl) piperidine | 1.11 | 451.2 | 452.4 |
| 79 | 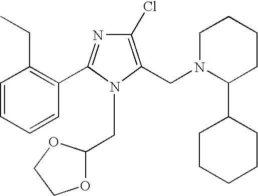 | 1-{[4-chloro-1-(1,3- dioxolan-2-ylmethyl)-2- (2-ethylphenyl)-1H- imidazol-5-yl]methyl}-2- cyclohexylpiperidine | 1.09 | 471.3 | 472.4 |
| 80 | 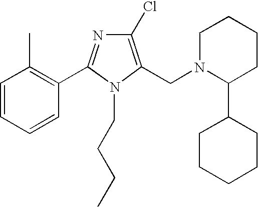 | 1-{[1-butyl-4-chloro-2- (2-methylphenyl)-1H- imidazol-5-yl]methyl}-2- cyclohexylpiperidine | 1.14 | 427.3 | 428.4 |
| 81 | 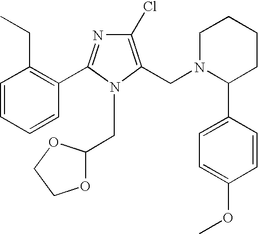 | 1-{[4-chloro-1-(1,3- dioxolan-2-ylmethyl)-2- (2-ethylphenyl)-1H- imidazol-5-yl]methyl}-2- (4-methoxyphenyl) piperidine | 1.08 | 495.2 | 496.4 |
| 82 | 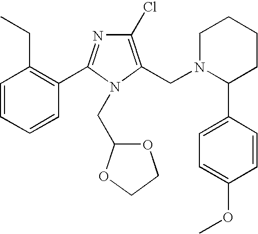 | 1-{[4-chloro-1-(1,3- dioxolan-2-ylmethyl)-2- (2-ethylphenyl)-1H- imidazol-5-yl]methyl}-2- (3,4-dimethoxyphenyl) piperidine | 1.06 | 525.2 | 526.5 |
| 83 | 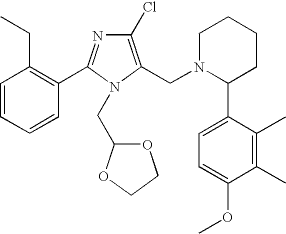 | 1-{[4-chloro-1-(1,3- dioxolan-2-ylmethyl)-2- (2-ethylphenyl)-1H- imidazol-5-yl]methyl}-2- (4-methoxy-2,3- dimethylphenyl) piperidine | 1.11 | 523.3 | 524.5 |
| 84 | 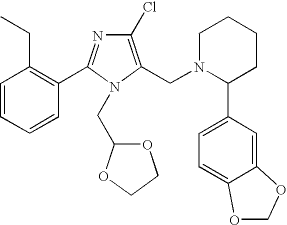 | 2-(1,3-benzodioxol-5-yl)- 1-{[4-chloro-1-(1,3- dioxolan-2-ylmethyl)-2- (2-ethylphenyl)-1H- imidazol-5-yl]methyl}piperidine | 1.12 | 509.2 | 510.2 |
| 85 | 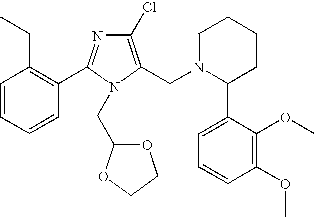 | 1-{[4-chloro-1-(1,3- dioxolan-2-ylmethyl)-2- (2-ethylphenyl)-1H- imidazol-5-yl]methyl}-2- (2,3-dimethoxyphenyl) piperidine | 1.12 | 525.2 | 526.2 |
| 86 | 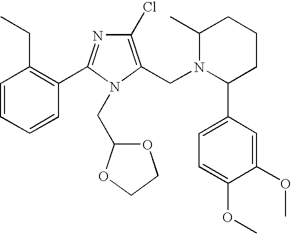 | 1-{[4-chloro-1-(1,3- dioxolan-2-ylmethyl)-2- (2-ethylphenyl)-1H- imidazol-5-yl]methyl}-2- (3,4-dimethoxyphenyl)- 6-methylpiperidine | 1.11 | 539.3 | 540.3 |
| 87 | 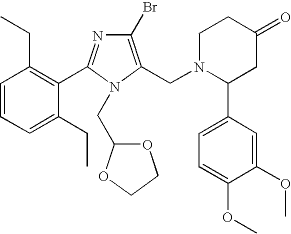 | 1-{[4-bromo-2-(2,6- diethylphenyl)-1-(1,3- dioxolan-2-ylmethyl)- 1H-imidazol-5- yl]methyl}-2-(3,4- dimethoxyphenyl) piperidin-4-one | | | |
| 88 | 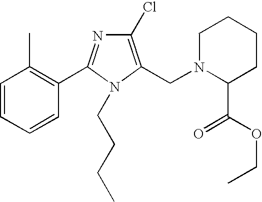 | ethyl 1-{[1-butyl-4- chloro-2-(2- methylphenyl)-1H- imidazol-5- yl]methyl}piperidine-2- carboxylate | 1.21 417.2 418.2 | | |
| 89 | 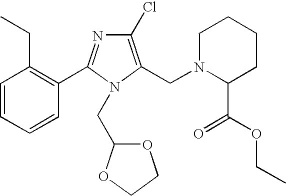 | ethyl 1-{[4-chloro-1- (1,3-dioxolan-2- ylmethyl)-2-(2- ethylphenyl)-1H- imidazol-5- yl]methyl}piperidine-2- carboxylate | 1.13 | 461.2 | 462.2 |
| 90 | 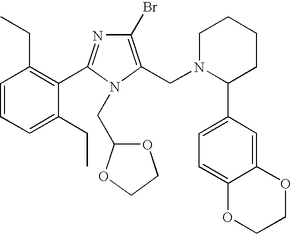 | 1-{[4-bromo-2-(2,6- diethylphenyl)-1-(1,3- dioxolan-2-ylmethyl)- 1H-imidazol-5- yl]methyl}-2-(2,3- dihydro-1,4-benzodioxin- 6-yl)piperidine | 1.15 | 595.2 | 596.2 |
| 91 | 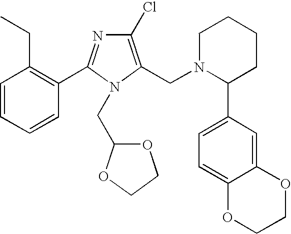 | 1-{[4-chloro-1-(1,3- dioxolan-2-ylmethyl)-2- (2-ethylphenyl)-1H- imidazol-5-yl]methyl}-2- (2,3-dihydro-1,4- benzodioxin-6- yl)piperidine | 1.11 | 523.2 | 524.2 |
| 92 | 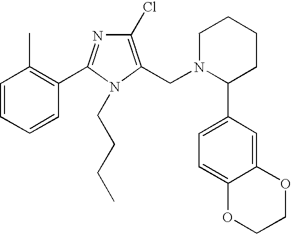 | 1-{[1-butyl-4-chloro-2- (2-methylphenyl)-1H- imidazol-5-yl]methyl}-2- (2,3-dihydro-1,4- benzodioxin-6- yl)piperidine | 1.16 | 479.2 | 480.2 |
| 93 | 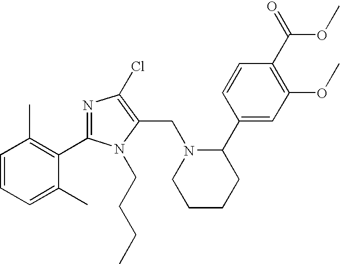 | methyl 4-(1-{[1-butyl-4- chloro-2-(2,6- dimethylphenyl)-1H- imidazol-5- yl]methyl}piperidin-2- yl)-2-methoxybenzoate | 1.18 | 523.3 | 524.3 |
| 94 | 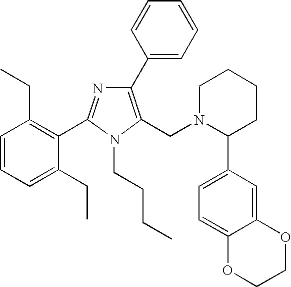 | 1-{[1-butyl-2-(2,6- diethylphenyl)-4-phenyl- 1H-imidazol-5- dihydro-1,4-benzodioxin- 6-yl)piperidine | 1.18 | 563.4 | 565.6 |
| 95 | 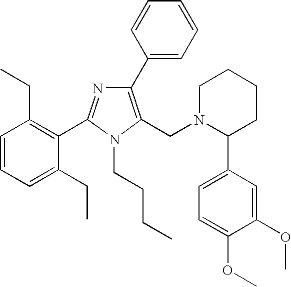 | 1-{[1-butyl-2-(2,6- diethylphenyl)4-phenyl- 1H-imidazol-5- yl]methyl}-2-(3,4- dimethoxyphenyl) piperidine | 1.15 | 565.4 | 566.6 |
| 96 | 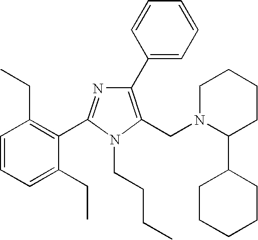 | 1-{[1-butyl-2-(2,6- diethylphenyl)-4-phenyl- 1H-iniidazol-5- yl]methyl}-2- cyclohexylpiperidine | 1.21 | 511.4 | 512.6 |
| 97 | 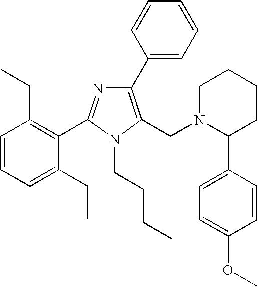 | 1-{[1-butyl-2-(2,6- diethylphenyl)-4-phenyl- 1H-imidazol-5- yl]methyl}-2-(4- ethoxyphenyl) piperidine | 1.18 | 535.4 | 536.6 |
| 98 | 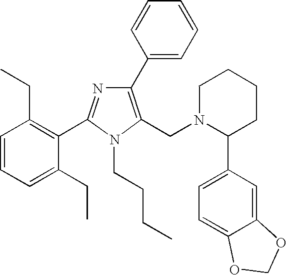 | 2-(1,3-benzodioxol-5-yl)- 1-{[1-butyl-2-(2,6- diethylphenyl)-4-phenyl- 1H-imidazol-5- yl]methyl}piperidine | 1.19 | 549.3 | 551.6 |
| 99 | 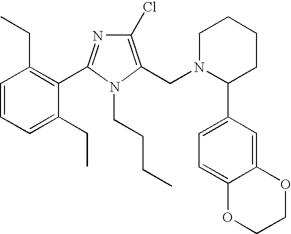 | 1-{[1-butyl-4-chloro-2- (2,6-diethylphenyl)-1H- imidazol-5-yl]methyl}-2- (2,3-dihydro-1,4- benzodioxin-6- yl)piperidine | 1.15 | 521.3 | 522.5 |
| 100 | 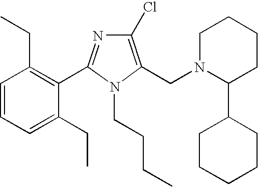 | 1-{[1-butyl-4-chloro-2- (2,6-diethylphenyl)-1H- imidazol-5-yl]methyl}-2- cyclohexylpiperidine | 1.19 | 469.3 | 470.5 |
| 101 | 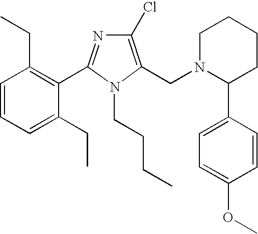 | 1-{[-butyl-4-chloro-2- (2,6-diethylphenyl)-1H- imidazol-5-yl]methyl)-2- (4-methoxyphenyl) piperidine | 1.16 | 493.3 | 494.5 |
| 102 | 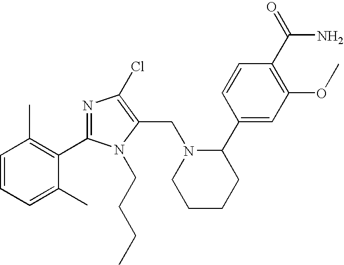 | 4-(1-{[1-butyl-4-chloro- 2-(2,6-dimethylphenyl)- 1H-imidazol-5- yl]methyl}piperidin-2- yl)-2-methoxybenzamide | 1.07 | 508.3 | 509.5 |
| 103 | 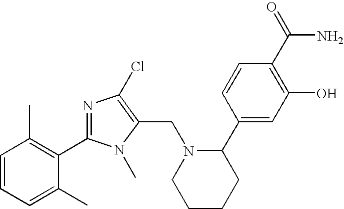 | 4-(1-{[4-chloro-2-(2,6- dimethylphenyl)-1- methyl-1H-imidazol-5- yl]methyl}piperidin-2- yl)-2-hydroxybenzamide | 1.01 | 452.2 | 453.4 |
| 104 | 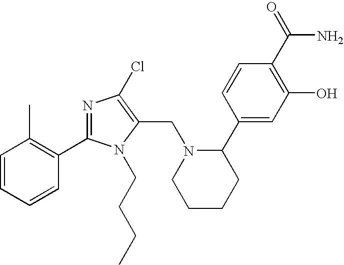 | 4-(1-{[1-butyl-4-chloro- 2-(2-methylphenyl)-1H- imidazol-5- yl]methyl}piperidin-2- yl)-2-hydroxybenzamide | 1.06 | 480.2 | 481.4 |
| 105 | 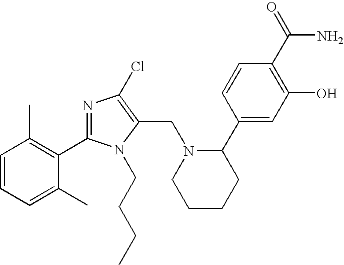 | 4-(1-{[1-butyl-4-chloro- 2-(2,6-dimethylphenyl)- 1H-imidazol-5- yl]methyl}piperidin-2- yl)-2-hydroxybenzamide | 1.08 | 494.2 | 495.5 |
| 106 | 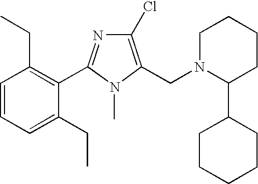 | 1-{[4-chloro-2-(2,6- diethylphenyl)-1-methyl- 1H-imidazol-5- yl]methyl}-2- cyclohexylpiperidine | 1.12 | 427.3 | 428.4 |
| 107 | 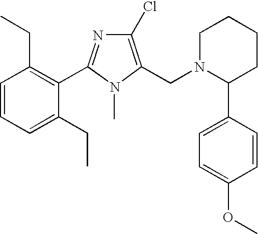 | 1-{[4-chloro-2-(2,6- diethylphenyl)-1-methyl- 1H-imidazol-5- yl]methyl}-2-(4- ethoxyphenyl) piperidine | 1.11 | 451.2 | 452.4 |
| 108 | 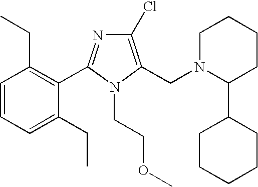 | 1-{[4-chloro-2-(2,6- diethylphenyl)-1-(2- methoxyethyl)-1H- imidazol-5-yl]methyl}-2- cyclohexylpiperidine | 1.13 | 471.3 | 472.5 |
| 109 | 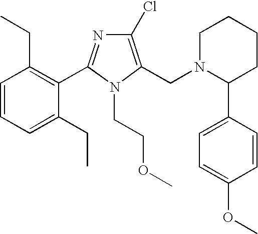 | 1-{[4-chloro-2-(2,6- diethylphenyl)-1-(2- methoxyethyl)-1H- imidazol-5-yl]methyl}-2- (4-methoxyphenyl) piperidine | 1.11 | 495.3 | 496.5 |
| 110 | 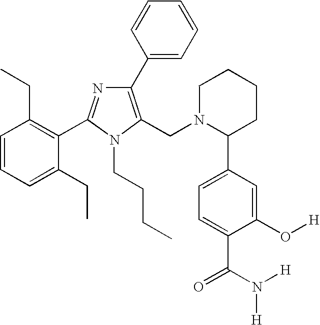 | 4-(1-{[1-butyl-2-(2,6- diethylphenyl)-4-phenyl- 1H-imidazol-5- yl]methyl}piperidin-2- yl)-2-hydroxybenzamide | 1.17 | 564.3 | 565.6 |
| 111 | 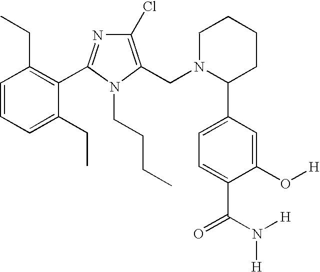 | 4-(1-{[4-chloro-2-(2,6- diethylphenyl)-1-methyl 1H-imidazol-5- yl]methyl}piperidin-2- yl)-2-hydroxybenzaxnide | 1.07 | 480.2 | 481.4 |
| 112 | 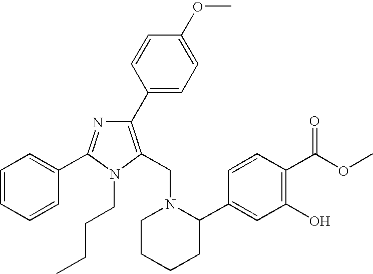 | methyl 4-(1-{[1-butyl-4- (4-methoxyphenyl)-2- phenyl-1H-imidazol-5- yl]methyl}piperidin-2- yl)-2-hydroxybenzoate | 1.25 | 553.3 | 554.3 |
| 113 | 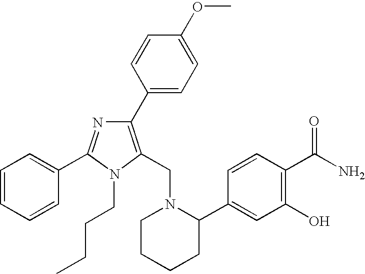 | 4-(1-{[1-butyl-4-(4- methoxyphenyl)-2- phenyl-1H-imidazol-5- yl]methyl}piperidin-2- yl)-2-hydroxybenzamide | 1.16 | 538.3 | 539.3 |
| 114 | 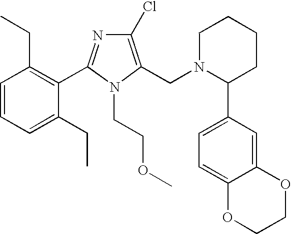 | 1-{[4-chloro-2-(2,6- diethylphenyl)-1-(2- methoxyethyl)-1H- imidazol-5-yl]methyl}-2- (2,3-dihydro-1,4- benzodioxin-6- yl)piperidine | 1.15 | 523.3 | 524.3 |
| 115 | 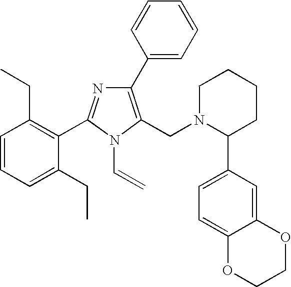 | 1-{[2-(2,6- diethylphenyl)-4-phenyl- 1-vinyl-1H-imidazol-5- 1]methyl}-2-(2,3- dihydro-1,4-benzodioxin- 6-yl)piperidine | 1.22 | 533.3 | 534.5 |
| 116 | 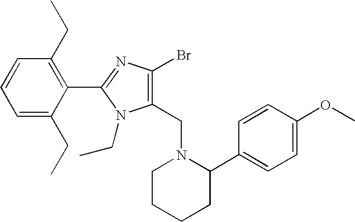 | 1-{[4-bromo-2-(2,6- diethylphenyl)-1-ethyl- 1H-imidazol-5- yl]methyl}-2-(4- methoxyphenyl) piperidine | 1.17 | 509.2 | 510.4 |
| 117 | 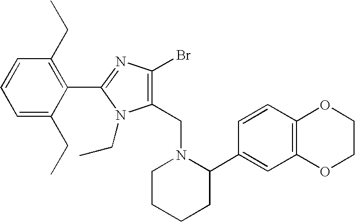 | 1-{[4-bromo-2-(2,6- diethylphenyl)-1-ethyl- 1H-imidazol-5- yl]methyl}-2-(2,3- dihydro-1,4-benzodioxin- 6-yl)piperidine | 1.16 | 537.2 | 538.5 |
| 118 | 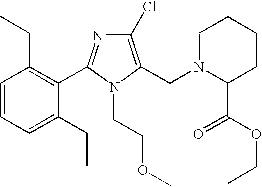 | ethyl 1-{[4-chloro-2- (2,6-diethylphenyl)-1-2- methoxyethyl)-1H- imidazol-5- yl]methyl}piperidine-2- carboxylate | 1.16 | 461.2 | 462.4 |
| 119 | 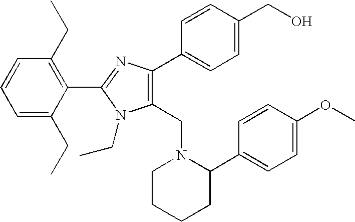 | [4-(2-(2,6- diethylphenyl)-1-ethyl-5- {[2-(4-methoxyphenyl) piperidin-1-yl]methyl}- 1H-imidazol-4- yl)phenyl]methanol | 1.16 | 537.3 | 538.6 |
| 120 | 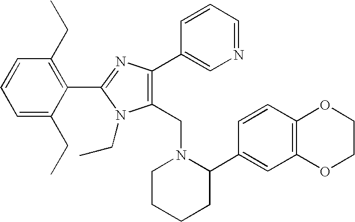 | 3-(2-(2,6-diethylphenyl)- 5-{[2-(2,3-dihydro-1,4- benzodioxin-6- yl)piperidin-1- 1]methyl}-1-ethyl-1H- imidazol-4-yl)pyridine | 1.16 | 536.3 | 537.6 |
| 121 | 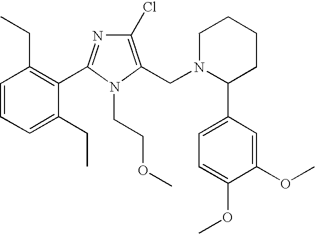 | 1-{[4-chloro-2-(2,6- diethylphenyl)-1-(2- methoxyethyl)-1H- imidazol-5-yl]methyl}-2- (3,4-dimethoxyphenyl) piperidine | 1.13 | 525.3 | 526.5 |
| 122 | 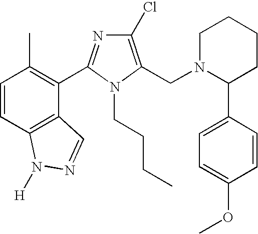 | 4-(1-butyl-4-chloro-5- {[2-(4-methoxyphenyl) piperidin-1-yl]methyl}- 1H-imidazol-2-yl)-5- methyl-1H-indazole | 1.12 | 491.2 | 492.5 |
| 123 | 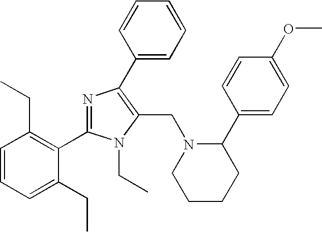 | 1-{[2-(2,6- diethylphenyl)-1-ethyl-4- phenyl-1H-imidazol-5- yl]methyl}-2-(4- methoxyphenyl) piperidine | 1.19 | 507.3 | 508.5 |
| 124 | 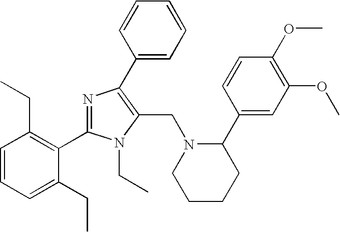 | 1-{[2-(2,6- diethylphenyl)-1-ethyl-4- phenyl-1H-imidazol-5- yl]methyl}-2-(3,4- dimethoxyphenyl) piperidine | 1.16 | 537.3 | 538.6 |
| 125 | 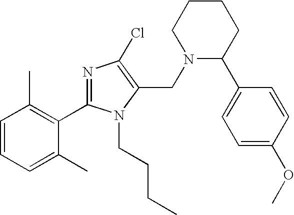 | 1-{[1-butyl-4-chloro-2- (2,6-dimethylphenyl)- 1H-imidazol-5- yl]methyl}-2-(4- methoxyphenyl) piperidine | 1.16 | 465.3 | 466.5 |
| 126 | 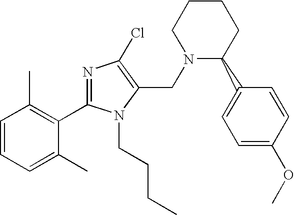 | (2S)-1-{[1-butyl-4- chloro-2-(2,6- dimethylphenyl)-1H- imidazol-5-yl]methyl)-2- (4-methoxyphenyl) piperidine | 1.16 | 465.3 | 466.4 |
| 127 | 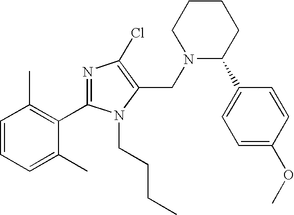 | (2R)-1-{[1-butyl-4- chloro-2-(2,6- dimethylphenyl)-1H imidazol-5-yl]methyl}-2- (4-methoxyphenyl) piperidine | 1.16 | 465.3 | 466.4 |
| 128 | 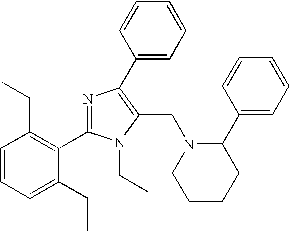 | 1-{[2-(2,6- diethyiphenyl)-1-ethyl-4- phenyl-1H-imidazol-5- yl]methyl}-2- phenylpiperidine | 1.21 | 477.3 | 478.5 |
| 129 | 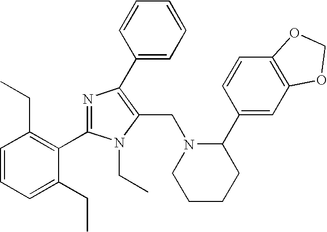 | 2-(1,3-benzodioxol-5-yl)- 1-{[2-(2,6- diethylphenyl)-1-ethyl-4- phenyl-1H-imidazol-5- yl]methyl}piperidine | 1.2 | 521.3 | 522.5 |
| 130 | 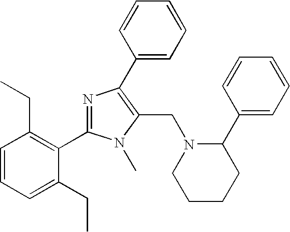 | 1-{[2-(2,6- diethylphenyl)-1-methyl- 4-phenyl-1H-imidazol-5- yl]methyl}-2- phenylpiperidine | 1.17 | 463.3 | 464.5 |
| 131 | 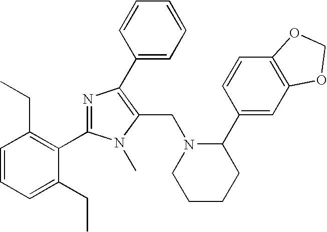 | 2-(1,3-benzodioxol-5-yl)- 1-{[2-(2,6- diethylphenyl)-1-methyl- 4-phenyl-1H-imidazol-5- yl]methyl}piperidine | 1.16 | 507.3 | 508.5 |
| 132 | 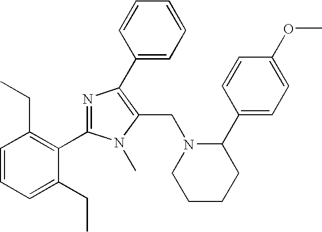 | 1-{[2-(2,6- diethylphenyl)-1-methyl- 4-phenyl-1H-imidazol-5- yl]methyl}-2-(4- methoxyphenyl) piperidine | 1.15 | 493.3 | 494.5 |
| 133 | 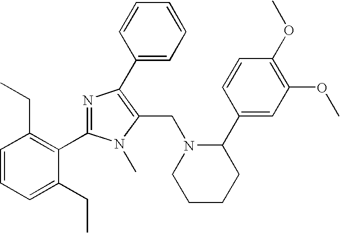 | 1-{[2-(2,6- diethylphenyl)-1-methyl- 4-phenyl-1H-imidazol-5- yl]methyl}-2-(3,4- dimethoxyphenyl) piperidine | 1.13 | 523.3 | 524.6 |
| 134 | 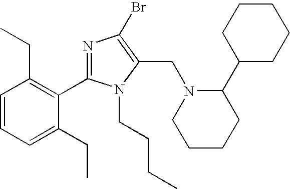 | 1-{[4-bromo-1-butyl-2- (2,6-diethylphenyl)-1H- imidazol-5-yl]methyl}-2- cyclohexylpiperidine | 1.26 | 513.3 | 514.3 |
| 135 | 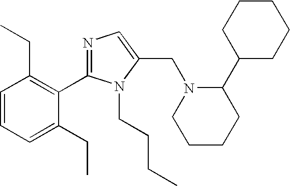 | 1-{[1-butyl-2-(2,6- diethylphenyl)-1H- imidazol-5-yl]methyl}-2- cyclohexylpiperidine | 1.17 | 435.4 | 436.5 |
| 136 | 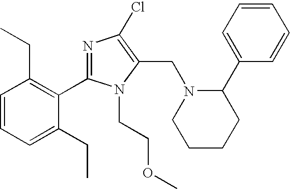 | 1-{[4-chloro-2-(2,6- diethylphenyl)-1-(2- methoxyethyl)-1H- imidazol-5-yl]methyl}-2- phenylpiperidine | 1.17 | 465.3 | 466.3 |
| 137 | 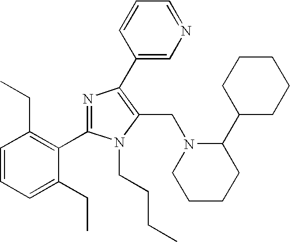 | 3-[1-butyl-5-[(2- cyclohexylpiperidin-1- yl)methyl]-2-(2,6- diethylphenyl)-1H- imidazol-4-yl]pyridine | 1.23 | 512.4 | 513.6 |
| 138 | 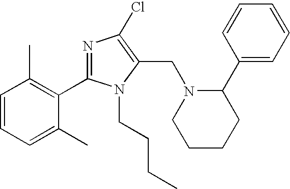 | 1-{[l-butyl-4-chloro-2- (2,6-dimethyiphenyl)- 1H-imidazol-5- yl]methyl}-2- phenylpiperidine | 1.19 | 435.2 | 436.4 |
| 139 | 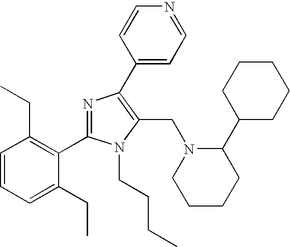 | 4-[1-butyl-5-[(2- cyclohexylpiperidin-1- yl)methyl]-2-(2,6- diethylphenyl)-1H- imidazol-4-yl]pyridine | 1.25 | 512.4 | 513.6 |
| 140 | 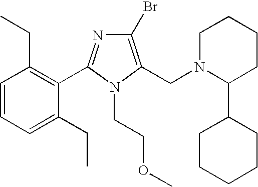 | 1-{[4-bromo-2-(2,6- diethylphenyl)-1-(2- methoxyethyl)-1H- imidazol-5-yl)methyl}-2- cyclohexylpiperidine | 1.18 | 515.3 | 516.5 |
| 141 | 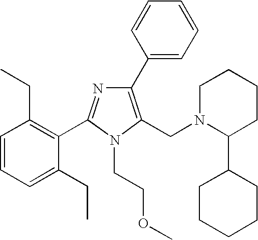 | 2-cyclohexyl-1-{[2-(2,6- diethyiphenyl)-1-(2- methoxyethyl)-4-phenyl- 1H-imidazol-5- yl]methyl}piperidine | 1.2 | 513.4 | 514.6 |
| 142 | 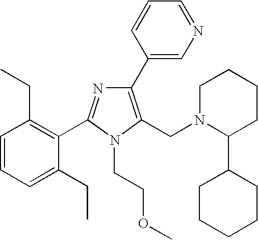 | 3-[5-[(2- cyclohexylpiperidin-1- yl)methyl]-2-(2,6- diethylphenyl)-1-(2- methoxyethyl)-1H- imidazol-4-yl]pyridine | 1.15 | 514.4 | 515.6 |
| 143 | 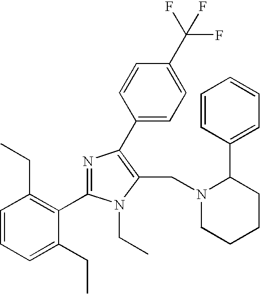 | 1-({2-(2,6- diethylphenyl)-1-ethyl-4- [4-(trifluoromethyl) phenyl)-1H-imidazol-5- yl}methyl)-2- phenylpiperidine | | | |
| 144 | 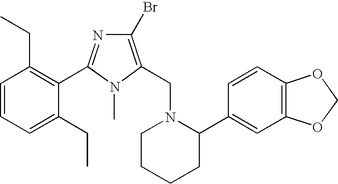 | 2-(1,3-benzodioxol-5-yl)- 1-{[4-bromo-2-(2,6- diethylphenyl)-1-methyl- 1H-imidazol-5- yl]methyl}piperidine | 1.17 | 509.2 | 510.2 |
| 145 | 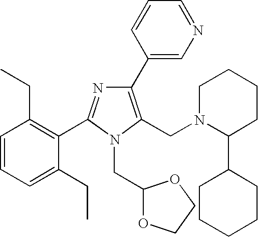 | 3-[5-[(2- cyclohexylpiperidin-1- yl)methyl]-2-(2,6- diethylphenyl)-1-(1,3- dioxolan-2-ylmethyl)- 1H-imidazol-4- yl]pyridine | 1.17 | 542.4 | 543.4 |
| 146 | 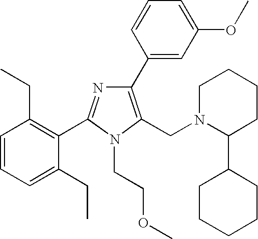 | 2-cyclohexyl-1-{[2-(2,6- diethylphenyl)-1-(2- methoxyethyl)-4-(3- methoxyphenyl)-1H- imidazol-5- yl]methyl}piperidine | 1.21 | 543.4 | 544.4 |
| 147 | 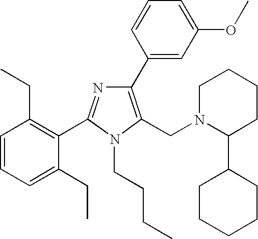 | 1-{[1-butyl-2-(2,6- diethylphenyl)-4-(3- methoxyphenyl)-1H- imidazol-5-yl]methyl}-2- cyclohexylpiperidine | 1.3 | 541.4 | 542.4 |
| 148 | 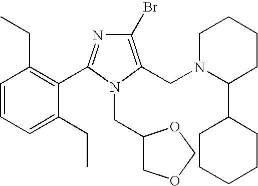 | 1-{[4-bromo-2-(2,6- diethylphenyl)-1-(1,3- dioxolan-2-ylmethyl)- 1H-imidazol-5- yl]methyl}-2- cyclohexylpiperidine | 1.19 | 543.2 | 544.2 |
| 149 | 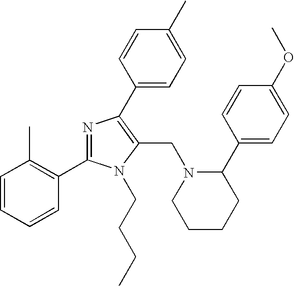 | 1-{[1-butyl-2-(2- methylphenyl)-4-(4- methylphenyl)-1H- imidazol-5-yl]methyl}-2- (4-methoxyphenyl) piperidine | 1.18 | 507.3 | 508.5 |
| 150 | 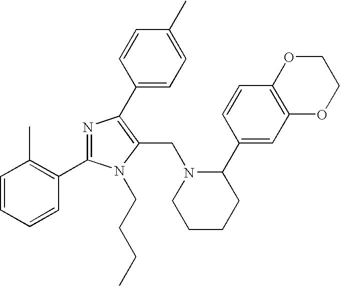 | 1-{[1-butyl-2-(2- methylphenyl)-4-(4- methylphenyl)-1H- imidazol-5-yl]methyl}-2- (2,3-dihydro-1,4- benzodioxin-6- yl)piperidine | 1.19 | 535.3 | 536.4 |
| 151 | 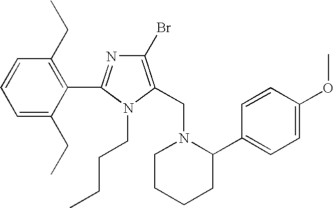 | 1-{[4-bromo-1-butyl-2- (2,6-diethylphenyl)-1H- imidazol-5-yl]methyl}-2- (4-methoxyphenyl) piperidine | | | |
| 152 | 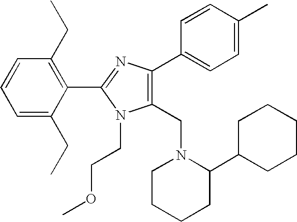 | 2-cyclohexyl-1-{[2-(2,6- diethylphenyl)-1-(2- methoxyethyl)-4-(4- methylphenyl)-1H- imidazol-5-yl]methyl}piperidine | 1.22 | 527.4 | 528.5 |
| 153 | 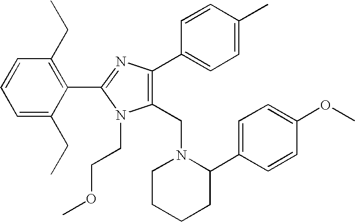 | 1-{[2-(2,6- diethylphenyl)-1-(2- methoxyethyl)-4-(4- methylphenyl)-1H- imidazol-5-yl]methyl}-2- (4-methoxyphenyl) piperidine | 1.18 | 551.4 | 553.5 |
| 154 | 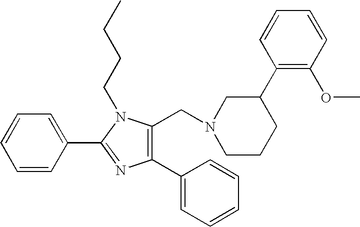 | 1-[(1-butyl-2,4-diphenyl- 1H-imidazol-5- yl)methyl]-3-(2- methoxyphenyl) piperidine | 1.15 | 479.3 | 480.2 |
| 155 | 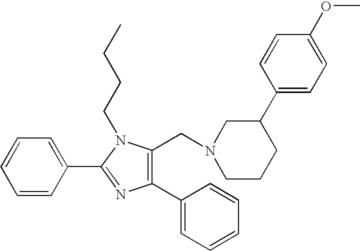 | 1-[(1-butyl-2,4-diphenyl- 1H-imidazol-5- yl)methyl]-3-(4- methoxyphenyl) piperidine | 1.18 | 479.3 | 480.2 |
| 156 | 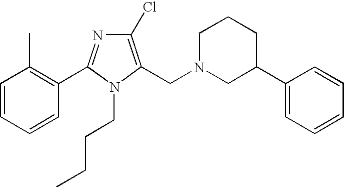 | 1-{[1-butyl-4-chloro-2- (2-methylphenyl)-1H- imidazol-5-yl]methyl}-3- phenylpiperidine | 1.11 | 421.2 | 422.4 |
| 157 | 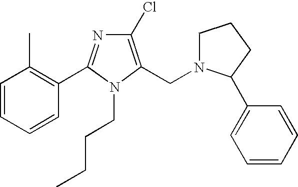 | 1-butyl-4-chloro-2-(2- methylphenyl)-5-[(2- phenylpyrrolidin-1- yl)methyl]-1H-imidazole | 1.1 | 407.2 | 408.3 |
| 158 | 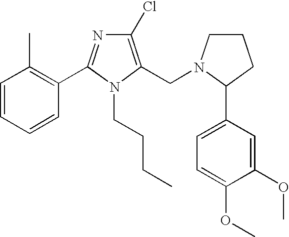 | 1-butyl-4-chloro-5-{[2- (3,4-dimethoxyphenyl) prrolidin-1-yl]methyl)- 2-(2-methylphenyl)-1H- imidazole | 1.08 | 467.2 | 468.4 |
| 159 | 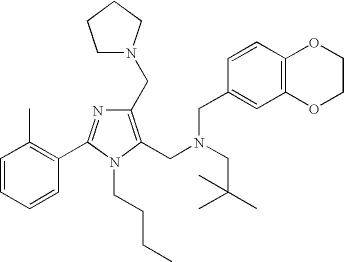 | N-{[1-butyl-2-(2- methylphenyl)-4- (pyrrolidin-1-ylmethyl)- 1H-imidazol-5- yl]methyl}-N-(2,3- dihydro-1,4-benzodioxin- 6-ylmethyl)-2,2- dimethylpropan-1-amine | 1.3 | 544.4 | 545.5 |
| 160 | 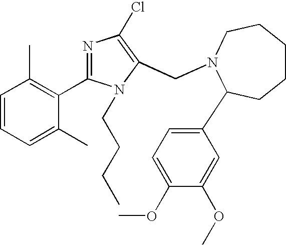 | 1-{[1-butyl-4-chloro-2- (2,6-dimethyiphenyl)- 1H-imidazol-5- yl]methyl}-2-(3,4- dimethoxyphenyl) azepane | 1.12 | 509.3 | 510.5 |
| 161 | 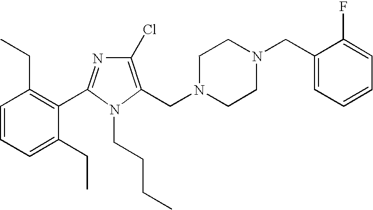 | 1-{[1-butyl-4-chloro-2- (2,6-diethylphenyl)-1H- imidazol-5-yl]methyl}-4- (2-fluorobenzyl) piperazine | 1.17 | 496.3 | 497.3 |
| 162 | 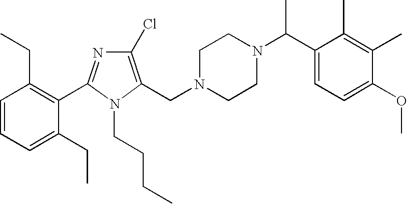 | 1-{[1-butyl-4-chloro-2- (2,6-diethylphenyl)-1H- imidazol-5-yl]methyl}-4- [1-(4-methoxy-2,3- dimethylphenyl)ethyl]piperazine | 1.2 | 550.3 | 551.6 |
| 163 | 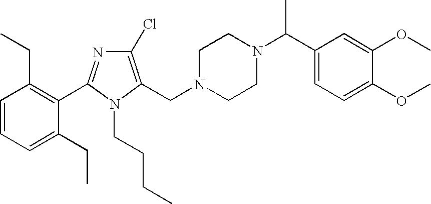 | 1-{[1-butyl-4-chloro-2- (2,6-diethylphenyl)-1H- imidazol-5-yl]methyl}-4- [1-(3,4- dimethoxyphenyl)ethyl]piperazine | 1.15 | 552.3 | 553.6 |
| 164 | 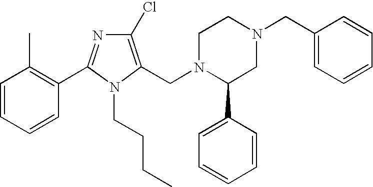 | (2R)-4-benzyl-1-{[1- butyl-4-chloro-2-(2- methylphenyl)-1H- midazol-5-yl]methyl}-2- phenylpiperazine | 1.23 | 512.3 | 513.3 |
| 165 | 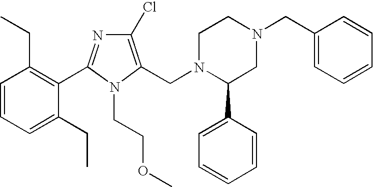 | (2R)-4-benzyl-1-{[4- chloro-2-(2,6- diethylphenyl)-1-(2- imidazol-5-yl]methyl}-2- phenylpiperazine | 1.21 | 556.3 | 557.6 |
| 166 | 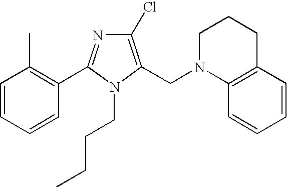 | 1-{[1-butyl-4-chloro-2- (2-methylphenyl)-1H- imidazol-5-yl]methyl}- 1,2,3,4- tetrahydroquinoline | 1.39 | 393.2 | 396.2 |
| 167 | 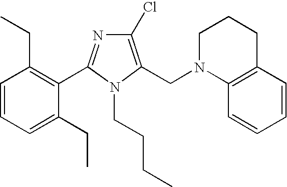 | 1-{[1-butyl-4-chloro-2- 2,6-diethylphenyl)-1H- imidazol-5-yl)methyl}- 1,2,3,4- tetrahydroquinoline | 1.4 | 435.2 | 436.4 |
| 168 | 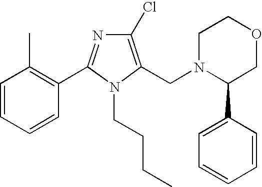 | (3R)-4-{[1-butyl-4- chloro-2-(2- methylphenyl)-1H- imidazol-5-yl]methyl)-3- phenylmorpholine | 1.26 | 423.2 | 424.3 |
| 169 | 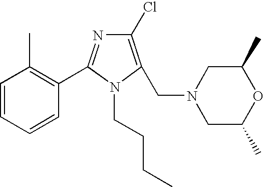 | (2R,6R)-4-{[1-butyl-4- chloro-2-(2- methylphenyl)-1H- imidazol-5-yl]methyl}- 2,6-dimethylmorpholine | 1.21 | 375.2 | 376.3 |
| 170 | 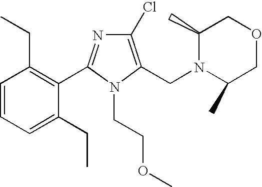 | (3R,5S)-4-{[4-chloro-2- (2,6-diethylphenyl)-1-(2- methoxyethyl)-1H- imidazol-5-yl]methyl}- 3,5-dimethylmorpholine | 1.11 | 419.2 | 420.4 |
| 171 | 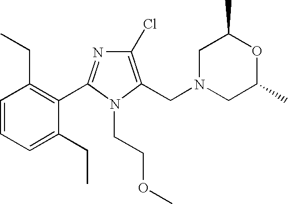 | (2R,6R)-4-{[4-chloro-2- (2,6-diethylphenyl)-1-(2- methoxyethyl)-1H- imidazol-5-yl]methyl}- 2,6-dimethylmorpholine | 1.17 | 419.2 | 420.4 |
| 172 | 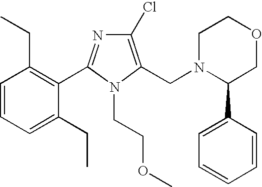 | (3R)-4-{[4-chloro-2-(2,6- diethylphenyl)-1-(2- ethoxyethyl)-1H- imidazol-5-yl]methyl}-3- phenylmorpholine | 1.28 | 467.2 | 468.4 |
| 173 | 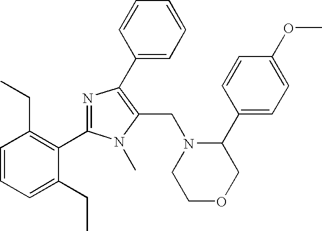 | diethylphenyl)-1-methyl- 4-phenyl-1H-imidazol-5- yl]methyl}-3-(4- methoxyphenyl) morpholine | 1.21 | 495.3 | 496.3 |
| 174 | 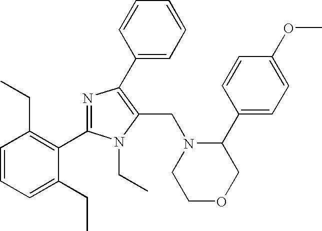 | 4-{[2-(2,6- diethylphenyl)-1-ethyl-4- phenyl-1H-imidazol-5- yl]methyl}-3-(4- methoxyphenyl) morpholine | 1.22 | 509.3 | 510.3 |
| 175 | 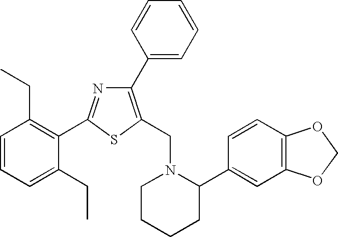 | 2-(1,3-benzodioxol-5-yl)- 1-{[2-(2,6- diethylphenyl)-4-phenyl 1,3-thiazol-5- yl]methyl}piperidine | 1.23 | 510.2 | 511.3 |
| 176 | 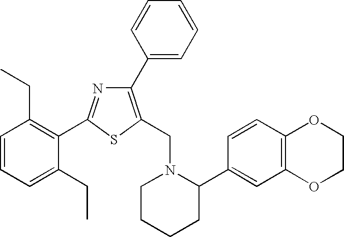 | 1-{[2-(2,6- diethylphenyl)-4-phenyl- 1,3-thiazol-5-yl]methyl}- 2-(2,3-dihydro-1,4- benzodioxin-6- yl)piperidine | 1.23 | 524.2 | 526.4 |
| 177 | 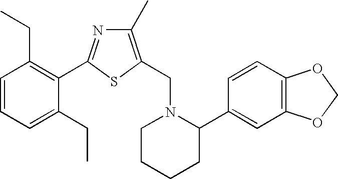 | 2-(1,3-benzodioxol-5-yl)- 1-{[2-(2,6- diethylphenyl)-4-methyl- 1,3-thiazol-5- yl]methyl}piperidine | 1.18 | 448.2 | 449.3 |
| 178 | 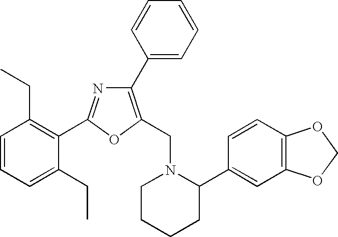 | 2-(1,3-benzodioxol-5-yl)- 1-{[2-(2,6- 1,3-oxazol-5- yl]methyl}piperidine | 1.22 | 494.3 | 495.3 |
| 179 | 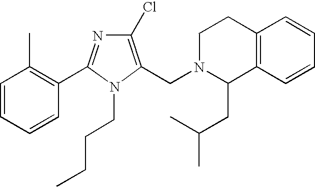 | 2-{[1-butyl-4-chloro-2- (2-methylphenyl)-1H- imidazol-5-yl]methyl}-1- isobutyl-1,2,3,4- tetrahydroisoquinoline | 1.24 | 449.3 | 450.5 |
| 180 | 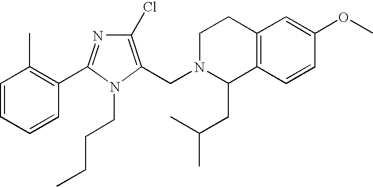 | 2-{[1-butyl-4-chloro-2- (2-methylphenyl)-1H- imidazol-5-yl]methyl}-1- isobutyl-6-methoxy- 1,2,3,4- tetrahydroisoquinoline | 1.25 | 479.3 | 480.3 |
| 181 | 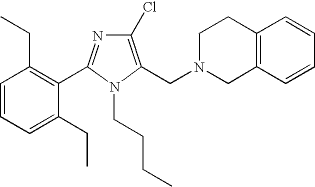 | 2-{[1-butyl-4-chloro-2- (2,6-diethylphenyl)-1H- imidazol-5-yl)methyl}- 1,2,3,4- tetrahydroisoquinoline | 1.13 | 435.2 | 434.3 |
| 182 | 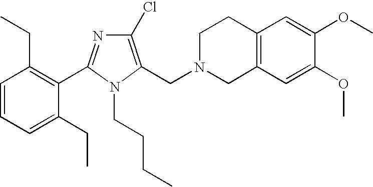 | 2-{[1-butyl-4-chloro-2- imidazol-5-yl]methyl}- 6,7-dimethoxy-1,2,3,4- tetrahydroisoquinoline | 1.15 | 495.3 | 496.3 |
| 183 | 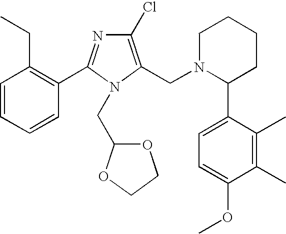 | 2-{[1-butyl-4-chloro-2- (2,6-diethylphenyl)-1H- imidazol-5-yl]methyl}-1- isobutyl-6-methoxy- 1,2,3,4- tetrahydroisoquinoline | 1.27 | 521.3 | 522.3 |
| 184 | 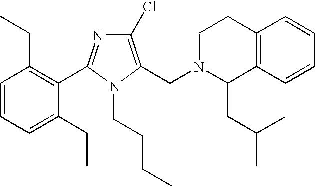 | 2-{[1-butyl-4-chloro-2- (2,6-diethylphenyl)-1H- imidazol-5-yl]methyl}-1- isobutyl- 1,2,3,4- tetrahydroisoquinoline | 1.15 | 491.3 | 490.3 |
| 185 | 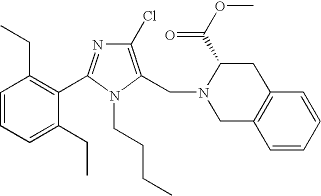 | methyl (3S)-2-{[1-butyl- 4-chloro-2-(2,6- diethylphenyl)-1H- imidazol-5-yl]methyl}- 1,2,3,4- tetrahydroisoquinoline-3- carboxylate | 1.15 | 493.2 | 492.3 |
| 186 | 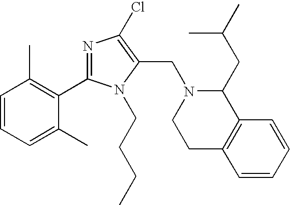 | 2-{[1-butyl-4-chloro-2- (2,6-dimethylphenyl)- 1H-imidazol-5- yl]methyl}-1-isobutyl- 1,2,3,4- tetrahydroisoquinoline | 1.32 | 463.3 | 464.3 |
| 187 | 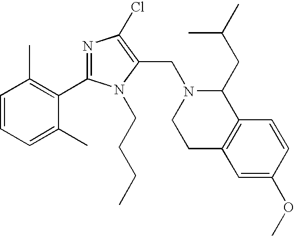 | 2-{[1-butyl-4-chloro-2- (2,6-dimethylphenyl)- 1H-imidazol-5- yl]methyl}isobutyl methoxy-l,2,3,4- tetrahydroisoquinoline | 1.28 | 493.3 | 494.3 |
| 188 | 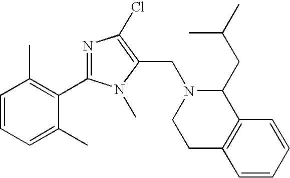 | 2-{[4-chloro-2-(2,6- dimethylphenyl)-1- methyl-1H-imidazol-5- yl]methyl}-1-isobutyl- 1,2,3,4- tetrahydroisoquinoline | 1.1 | 421.2 | 420.2 |

TABLE 2

| | | | Ret. | LCMS | LCMS | |
|---|---|---|---|---|---|---|
| | Compound | Name | Time | Mass | M + H | Protocol |
| 189 | 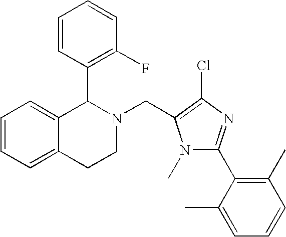 | 2-{[4-chloro-2-(2,6- dimethylphenyl)-1-methyl- 1H-imidazol-5- yl]methyl}-1-(2- fluorophenyl)-1,2,3,4- tetrahydroisoquinoline | 1.28 | 459.2 | 460.3 | A |
| 190 | 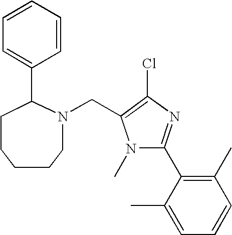 | 1-{[4-chloro-2-(2,6- dimethylphenyl)-1-methyl- 1H-imidazol-5- yl]methyl}-2- phenylazepan | 1.08 | 407.2 | 408.3 | A |
| 191 | 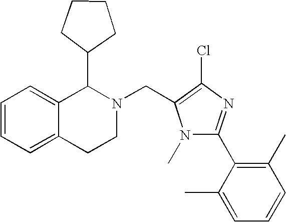 | 2-{[4-chloro-2-(2,6- dimethylphenyl)-1-methyl- 1H-imidazol-5- yl]methyl}-1-cyclopentyl- 1,2,3,4- tetrahydroisoquinoline | 1.16 | 433.2 | 434.2 | A |
| 192 | 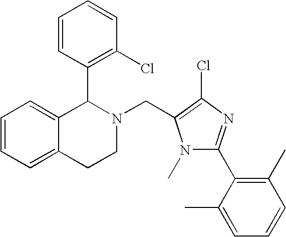 | 2-{[4-chloro-2-(2,6- dimethylphenyl)-1-medthyl- 1H-imidazol-5- yl]methyl}-1-(2- chlorophenytl)-1,2,3,4- tetrahydroisoquinoline | 1.3 | 475.2 | 476.2 | A |
| 193 | 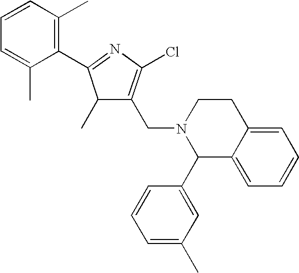 | 2-{[4-chloro-2-(2,6- dimethylphenyl)-1-medthyl- 1H-imidazolo-5- yl]methyl}-1-(3- methylphenyl)-1,2,3,4- tetrahydroisoquinoline | 1.23 | 455.21 | 456.2 | A |
| 194 | 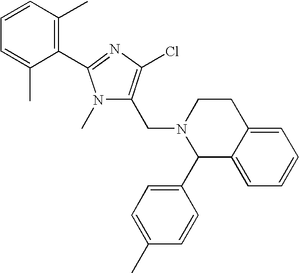 | 2-{[4-chloro-2-(2,6- dimethylphenyl)-1-methyl- 1H-imidazol-5- yl]methyl}-2-(4- methylphenyl)-1,2,3,4- tetrahydroisoquinoline | 1.22 | 455.2 | 456.2 | A |
| 195 | 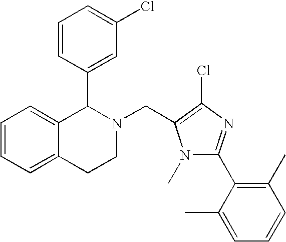 | 2-{[4-chloro-2-(2,6- dimethylphenyl)-1-methyl- 1H-imidazol-5- yl]methyl}-1-(3- chlorophenyl)-1,2,3,4- tetrahydroisoquinoline | 1.33 | 475.2 | 476.2 | A |
| 196 | 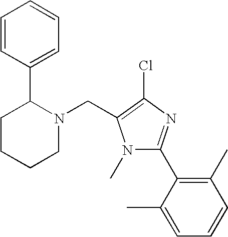 | 1-{[4-chloro-2-(2,6- dimethylphenyl)-1-methyl- 1H-imidazol-5- yl]methyl}-2- phenylpiperidine | 1.06 | 393.2 | 394.2 | A |
| 197 | 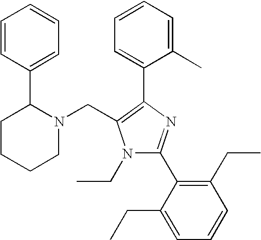 | 1-{[2-(2,6-diethylphenyl)- 1-ethyl-4-(2- methylphenyl)-1H- imidazol-5-yl]methyl}-2- phenylpiperidine | 1.26 | 491.3 | 492.5 | B |
| 198 | 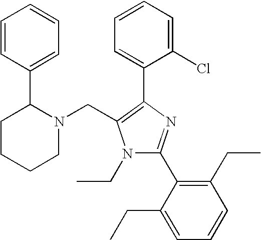 | 1-{[4-(2-chlorophenyl)-2- (2,6-diethylphenyl)-1- ethyl-1H-imidazol-5- yl]methyl}-2- phenylpiperidine | 1.27 | 511.3 | 512.5 | B |
| 199 | 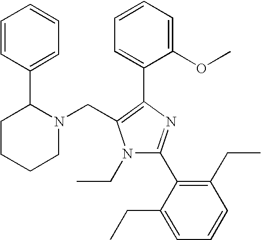 | 1-{[2-(2,6-diethylphenyl)- 1-ethyl-4-(2- methoxyphenyl)-1H- imidazol-5-yl]methyl}-2- phenylpiperidine | 1.24 | 507.3 | 508.5 | B |
| 200 | 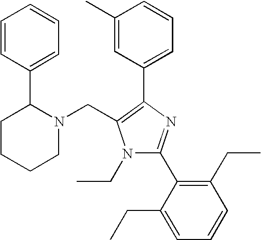 | 1-{[2-(2,6-diethylphenyl)- 1-ethyl-4-(3- methylphyenyl)-1H- imidazol-5-yl]methyl}-2- phenylpiperidine | 1.25 | 491.3 | 492.5 | B |
| 201 | 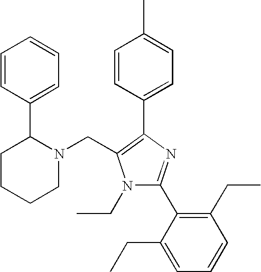 | 1-{[2-(2,6-diethylphenyl)- 1-ethyl-4-(4- methylphenyl)-1H- imidazol-5-yl]methyl}-2- phenylpiperidine | 1.25 | 491.3 | 492.5 | B |
| 202 | 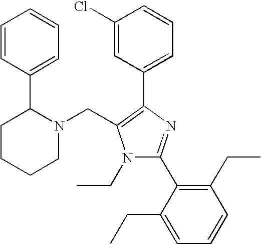 | 1-{[4-(3-chlorpophenyl)-2- (2,6-diethylphenyl)-1- ethyl-1H-imidazol-5- yl]methyl}-2- phenylpiperidine | 1.27 | 511.3 | 512.5 | B |
| 203 | 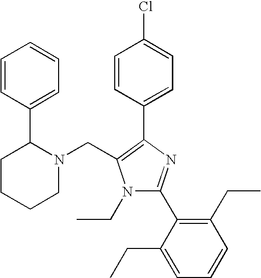 | 1-{[4-(4-chlorophenyl)-2- (2,6-diethylphenyl)-1- ethyl-1H-imidazol-5- yl]methyl}-2- phenylpiperidine | 1.26 | 511.3 | 512.5 | B |
| 204 | 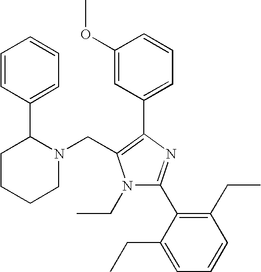 | 1-{[2-(2,6-diethylphenyl)- 1-ethyl-4-(3- methoxyphenyl)-1H- imidazol-5-yl]methyl}-2- phenylpiperidine | 1.25 | 507.3 | 508.5 | B |
| 205 | 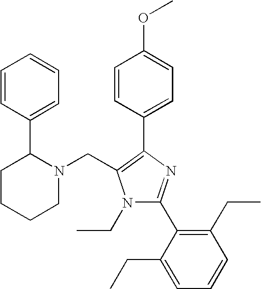 | 1-{[2-(2,6-diethylphenyl)- 1-ethyl-4-(4- methoxyphenyl)-1H- imidazol-5-yl]methyl}-2- phenylpiperidine | 1.24 | 507.3 | 508.5 | B |
| 206 | 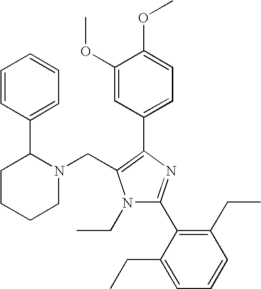 | 1-{[2-(2,6-diethylphenyl)- 4-(3,4-dimethoxyphenyl)- 1-ethyl-1H-imidazol-5- yl]methyl}-2- phenylpiperidine | | | | B |
| 207 | 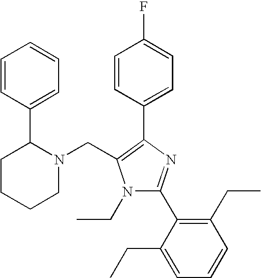 | 1-{[2-(2,6-diethylphenyl)- 1-ethyl-4-(4- fluorophenyl)-1H- imidazol-5-yl]methyl}-2- phenylpiperidine | 1.23 | 495.3 | 496.5 | B |
| 208 | 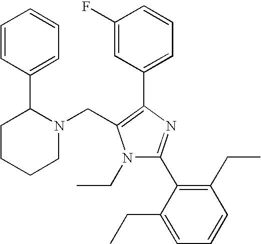 | 1-{[2-(2,6-diethylphenyl)- 1-ethyl-4-(3- fluorophenyl)-1H- imidazol-5-yl]methyl}-2- phenylpiperidine | 1.25 | 495.3 | 496.5 | B |
| 209 | 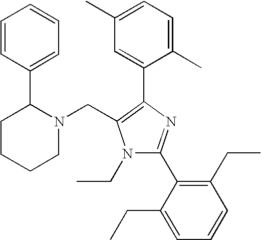 | 1-{[2-(2,6-diethylphenyl)- 4-(2,5-dimethylphenyl)-1- ethyl-1H-imidazol-5- yl]methyl}-2- phenylpiperidine | 1.27 | 505.3 | 506.5 | B |
| 210 | 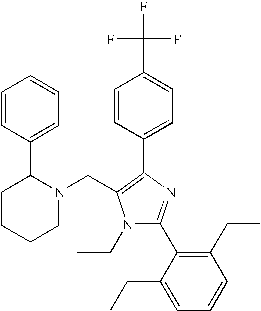 | 1-({2-(2,6-diethylphenyl)- 1-ethyl-4-[4- (trifluoromethyl)phenyl]- 1H-imidazol-5- yl}methyl)-2- phenylpiperidine | 1.28 | 545.3 | 546.5 | B |
| 211 | 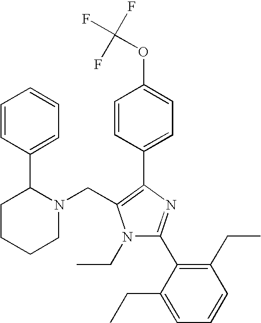 | 1-({2-(2,6-diethylphenyl)- 1-ethyl-4-[4- (trifluoromethoxy)phenyl]- 1H-imidazol-5- yl}methyl)-2- phenylpiperidine | 1.28 | 561.3 | 562.5 | B |
| 212 | 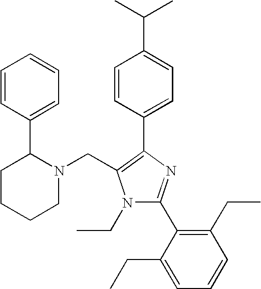 | 1-{[2-(2,6-diethylphenyl)- 1-ethyl-4-(4- isopropylphenyl)-1H- imidazol-5-yl]methyl}-2- phenylpiperidine | 1.29 | 519.4 | 520.6 | B |
| 213 | 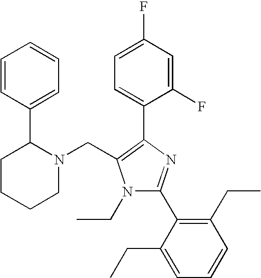 | 1-{[2-(2,6-diethylphenyl)- 4-(2,4-difluorophenyl)-1- ethyl-1H-imidazol-5- yl]methyl}-2- phenylpiperidine | 1.26 | 513.3 | 514.5 | B |
| 214 | 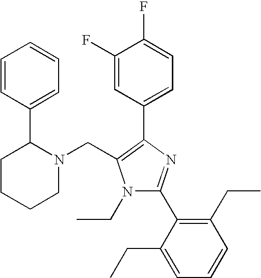 | 1-{[2-(2,6-diethylphenyl)- 4-(3,4-difluorophenyl)-1- ethyl-1H-imidazol-5- yl]methyl}-2- phenylpiperidine | 1.26 | 513.3 | 514.5 | B |
| 215 | 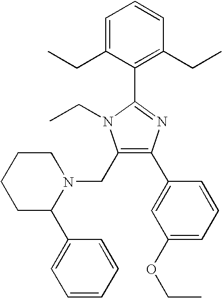 | 1-{[2-(2,6-diethylphenyl)- 4-(3-ethoxyphenyl)-1- ethyl-1H-imidazol-5- yl]methyl}-2- phenylpiperidine | 1.26 | 521.3 | 522.6 | B |
| 216 | 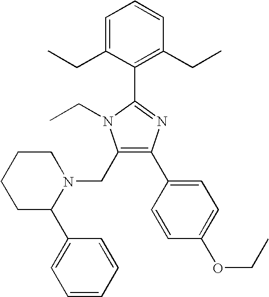 | 1-{[2-(2,6-diethylphenyl)- 4-(4-ethoxyphenyl)-1- ethyl-1H-imidazol-5- yl]methyl}-2- phenylpiperidine | 1.26 | 521.3 | 522.6 | B |
| 217 | 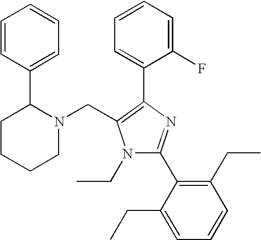 | 1-{[2-(2,6-diethylphenyl)- 1-ethyl-4-(2- fluorophenyl)-1H- imidazol-5-yl]methyl}-2- phenylpiperidine | 1.25 | 495.3 | 496.5 | B |
| 218 | 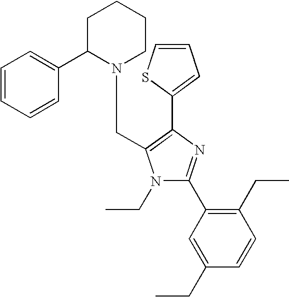 | 1-{[2-(2,6-diethylphenyl)- 1-ethyl-4-(2-thienyl)-1H- imidazol-5-yl]methyl}-2- phenylpiperidine | 1.23 | 483.3 | 484.5 | B |
| 219 | 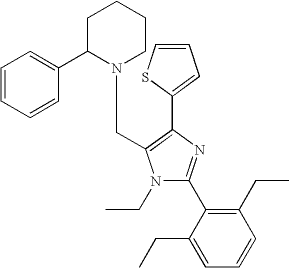 | 1-{[2-(2,6-diethylphenyl)- 1-ethyl-4-(3-thienyl)-1H- imidazol-5-yl]methyl}-2- phenylpiperidine | 1.21 | 483.3 | 484.5 | B |
| 220 | 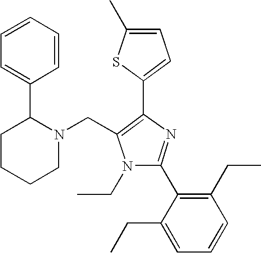 | 1-{[2-(2,6-diethylphenyl)- 1-ethyl-4-(5-methyl-2- thienyl)-1H-imidazol-5- yl]methyl}-2- phenylpiperidine | | | | B |
| 221 | 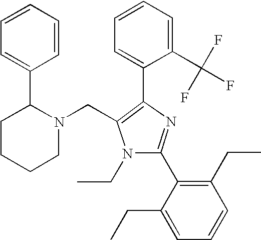 | 1-({2-(2,6-diethylphenyl)- 1-ethyl-4-[2- (trifluoromethyl)phenyl]- 1H-imidazol-5- yl}methyl)-2- phenylpiperidine | 1.28 | 545.3 | 546.5 | B |
| 222 | 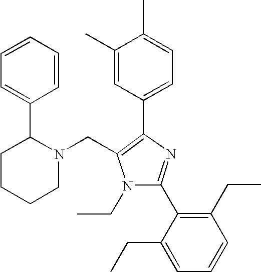 | 1-{[2-(2,6-diethylphenyl)- 4-(3,4-dimethylphenyl)-1- ethyl-1H-imidazol-5- yl]methyl}-2- phenylpiperidine | 1.28 | 505.3 | 506.5 | B |
| 223 | 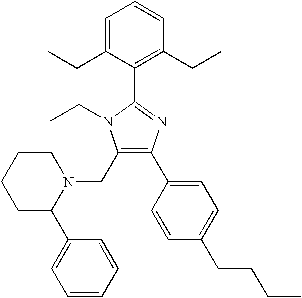 | 1-{[4-(4-butylphenyl)-2- (2,6-diethylphenyl)-1- ethyl-1H-imidazol-5- yl]methyl}-2- phenylpiperidine | 1.32 | 533.4 | 534.6 | B |
| 224 | 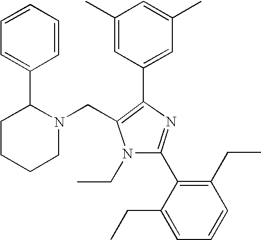 | 1-{[2-(2,6-diethylphenyl)- 4-(3,5-dimethylphenyl)-1- ethyl-1HH-imidazol-5- yl]methyl}-2- phenylpiperidine | 1.27 | 505.3 | 506.6 | B |
| 225 | 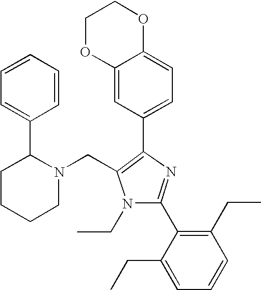 | 1-{[2-(2,6-diethylphenyl)- 4-(2,3-dihydro-1,4- benzodioxin-6-yl)-1-ethyl- 1H-imidazol-5- yl]methyl}-2- phenylpiperidine | 1.23 | 535.3 | 536.6 | B |
| 226 | 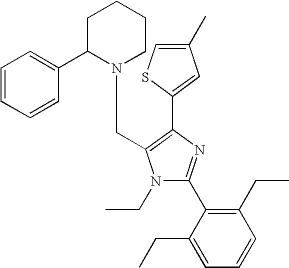 | 1-{[2-(2,6-diethylphenyl)- 1-ethyl-4-(4-methyl-23- thienyl)-1H-imidazol-5- yl]methyl}-2- phenylpiperidine | 1.26 | 497.3 | 498.5 | B |
| 227 | 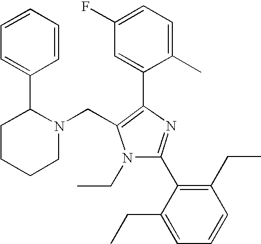 | 1-{[2-(2,6-diethylphenyl)- 1-ethyl-4-(5-fluoro-2- methylphenyl)-1H- imidazol-5-yl]methyl}-2- phenylpiperidine | 1.26 | 509.3 | 510.5 | B |
| 228 | 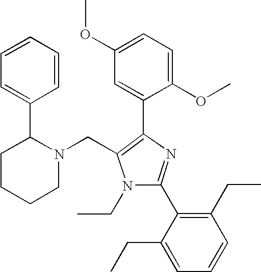 | 1-{[2-(2,6-diethylphenyl)- 4-(2,5-dimethoxyphenyl)- 1-ethyl-1H-imidazol-5- yl]methyl}-2- phenylpiperidine | 1.25 | 537.3 | 538.6 | B |
| 229 | 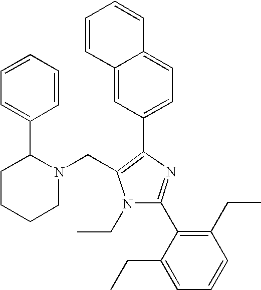 | 1-{[2-(2,6-diethylphenyl)- 1-ethyl-4-(2-naphthyl)-1H- imidazol-5-yl]methyl}-2- phenylpiperidine | 1.28 | 527.3 | 528.6 | B |
| 230 | 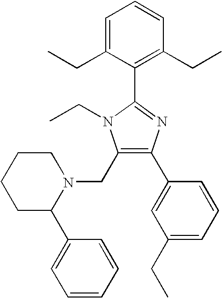 | 1-{[2-(2,6-diethylphenyl)- 1-ethyl-4-(3-ethylphenyl)- 1H-imidazol-5- yl]methyl}-2- phenylpiperidine | 1.27 | 505.3 | 506.5 | B |
| 231 | 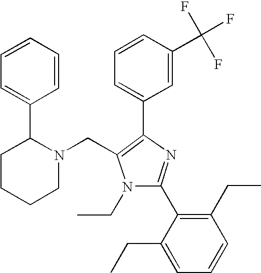 | 1-({2-(2,6-diethylphenyl)- 1-ethyl-44-[3- (trifluoromethyl)phenyl]- 1H-imidazol-5- yl}methyl)-2- phenylpiperidine | 1.28 | 545.3 | 546.5 | B |
| 232 | 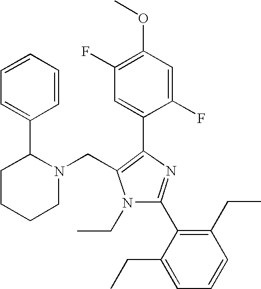 | 1-{[2-(2,6-diethylphenyl)- 4-(2,5-difluoro-4- methoxyphenyl)-1-ethyl- 1H-imidazol-5- yl]methyl}-2- phenylpiperidine | 1.25 | 543.3 | 544.6 | B |
| 233 | 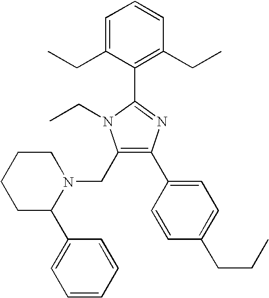 | 1-{[2-(2,6-diethylphenyl)- 1-ethyl-4-(4- propylphenyl)-1H- imidazol-5-yl]methyl}-2- phenylpiperidine | 1.3 | 519.4 | 520.6 | B |
| 234 | 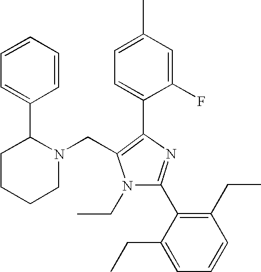 | 1-{[2-(2,6-diethylphenyl)- 1-ethyl-4-(2-fluoro-4- methylphenyl)-1H- imidazol-5-yl]methyl}-2- phenylpiperidine | 1.27 | 509.3 | 510.5 | B |
| 235 | 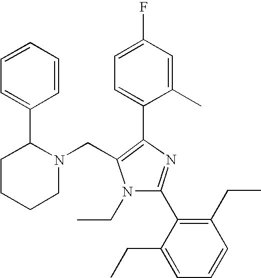 | 1-{[2-(2,6-diethylphenyl)- 1-ethyl-4-(4-fluoro-2- methylphenyl)-1H- imidazol-5-yl]methyl}-2- phenylpiperidine | 1.26 | 509.3 | 510.5 | B |
| 236 | 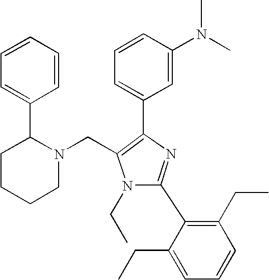 | 3-{2-(2,6-diethylphenyl)- 1-ethyl-5-[(2- phenylpiperidin-1- yl)methyl]-1H-imidazol-4- yl}-N,N-dimethylammine | 1.24 | 520.4 | 521.6 | B |
| 237 | 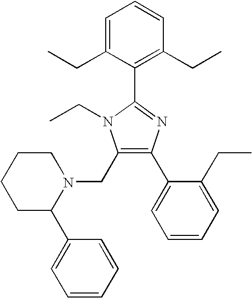 | 1-{[2-(2,6-diethylphenyl)- 1-ethyl-4-(2-ethylphenyl)- 1H-imidazol-5- yl]methyl}-2- phenylpiperidine | 1.28 | 505.3 | 506.6 | B |
| 238 | 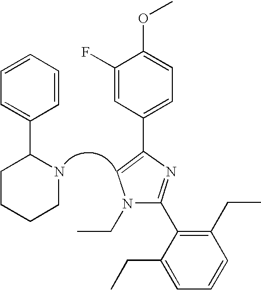 | 1-{[2-(2,6-diethylphenyl)- 1-ethyl-4-(3-fluoro-4- methoxyphenyl)-1H- imidazol-5-yl]methyl}-2- phenylpiperidine | 1.24 | 525.3 | 526.5 | B |
| 239 | 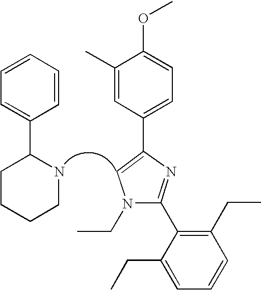 | 1-{[2-(2,6-diethylphenyl)- 1-ethyl-4-(4-methyl-3- methylphenyl)-1H- imidazol-5-yl]methyl}-2- phenylpiperidine | 1.25 | 521.3 | 522.6 | B |
| 240 | 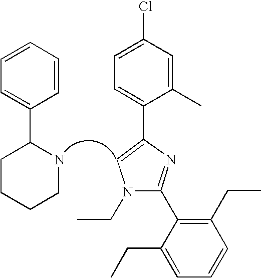 | 1-{[4-(4-chloro-2- methylphenyl)-2-(2,6- diethylphenyl)-1-ethyl-1H- imidazol-5-yl]methyl}-2- phenylpiperidine | 1.29 | 525.3 | 526.5 | B |

### EXAMPLE 7

### Pharmaceutical Preparations of Oral and Intravenous Administration

A. Tablets containing a C5a antagonist and an anti-arthritic agent which is not a C5a receptor antagonist can be prepared as illustrated below:

| Ingredient | Amount |
|---|---|
| C5a receptor antagonist | 5 mg-500 mg |
| C5a receptor-inactive therapeutic agent | 1 mg-500 mg |
| diluent, binder, disintigrant, lubricant excipients | q.s. 200-400 mg. |

B. Tablets containing a C5a receptor antagonist as the only active ingredient can be prepared as illustrated below:

| Ingredient | mg | mg |
|---|---|---|
| C5a receptor antagonist | 10 | 50 |
| Microcrystalline Cellulose | 70.4 | 352 |
| Grannular Mannitol | 15.1 | 75.5 |
| Croscarmellose Sodium | 3.0 | 15.0 |
| Colloidal Silicon Dioxide | 0.5 | 2.5 |
| Magnesium Stearate (Impalpable Powder) | 1.0 | 5.0 |
| Total (mg) | 100 | 500 |

C. Tablets containing a C5a receptor antagonist and a C5a receptor inactive agent may be prepared as follows:

| Ingredient | mg | mg |
|---|---|---|
| C5a receptor antagonist | 10 | 25 |
| C5a receptor inactive therapeutic agent | 10 | 25 |
| Microcrystalline Cellulose | 40 | 100 |
| Modified food corn starch | 1.05 | 4.25 |
| Magnesium stearate | 1.25 | 0.5 |

D. Intravaneous formulations containing a C5a receptor antagonist and a C5a receptor inactive agent may be prepared as follows:

| Ingredient | Amount | |
|---|---|---|
| C5a receptor antagonist | 0.5-10 | mg |
| C5a receptor inactive therapeutic agent | 0.5-10 | mg |
| Sodium Citrate | 5-50 | mg |
| Citric Acid | 1-15 | mg |
| Sodium Chloride | 1-8 | mg |
| Water for Injection | to 1.0 | liter |

E. Oral suspensions containing a C5a receptor antagonist and a C5a receptor inactive agent may be prepared as follows:

| Ingredient | Amount per 5 ml dose | |
|---|---|---|
| C5a receptor antagonist | 5-100 | mg |
| C5a receptor inactive therapeutic agent | 5-100 | mg |
| Polyvinylpyrrolidone | 150 | mg |
| Poly oxyethylene sorbitan monolaurate | 25 | mg |

| | |
|---|---|
| Benzoic Acid | 10 mg to 5 mL with |
| | sorbitol solution (70%) |

### EXAMPLE 8

### Preparation of Radiolabeled Probe Compounds

Compounds provided herein are prepared as radiolabeled probes by carrying out their synthesis using precursors comprising at least one atom that is a radioisotope. The radioisotope is preferably selected from of at least one of carbon (preferably ¹⁴C), hydrogen (preferably ³H), sulfur (preferably ³⁵S), or iodine (preferably ¹²⁵I). Such radiolabeled probes are conveniently synthesized by a radioisotope supplier specializing in custom synthesis of radiolabeled probe compounds. Such suppliers include Amersham Corporation, Arlington Heights, Ill.; Cambridge Isotope Laboratories, Inc. Andover, Mass.; SRI International, Menlo Park, Calif.; Wizard Laboratories, West Sacramento, Calif.; ChemSyn Laboratories, Lexena, Kans.; American Radiolabeled Chemicals, Inc., St. Louis, Mo.; and Moravek Biochemicals Inc., Brea, Calif.

Tritium labeled probe compounds are also conveniently prepared catalytically via platinum-catalyzed exchange in tritiated acetic acid, acid-catalyzed exchange in tritiated trifluoroacetic acid, or heterogeneous-catalyzed exchange with tritium gas. Such preparations are also conveniently carried out as a custom radiolabeling by any of the suppliers listed in the preceding paragraph using a compound provided herein as substrate. In addition, certain precursors may be subjected to tritium-halogen exchange with tritium gas, tritium gas reduction of unsaturated bonds, or reduction using sodium borotritide, as appropriate.

### EXAMPLE 9

### Receptor Autoradiography

Receptor autoradiography (receptor mapping) is carried out in vitro as described by Kuhar in sections 8.1.1 to 8.1.9 of Current Protocols in Pharmacology (1998) John Wiley & Sons, New York, using radiolabeled compounds prepared as described herein.

### EXAMPLE 10

### Assay for C5a Receptor Mediated Chemotaxis

This assay is a standard assay of C5a receptor mediated chemotaxis.

Human promonocytic U937 cells or purified human or non-human neutrophils are treated with dibutyryl cAMP for 48 hours prior to performing the assay. Human neutrophils or those from another mammalian species are used directly after isolation. The cells are pelleted and resuspended in culture media containing 0.1% fetal bovine serum (FBS) and 10 μg/ml calcein AM (a fluorescent dye). This suspension is then incubated at 37° C. for 30 minutes such that the cells take up the fluorescent dye. The suspension is then centrifuged briefly to pellet the cells, which are then resuspended in culture media containing 0.1% FBS at a concentration of approximately 3×10⁶ cells/mL. Aliquots of this cell suspension are transferred to clean test tubes, which contain vehicle (1% DMSO) or varying concentrations of a compound of interest, and incubated at room temperature for at least 30 minutes. The chemotaxis assay is performed in CHEMO TX 101-8, 96 well plates (Neuro Probe, Inc. Gaithersburg, Md.). The bottom wells of the plate are filled with medium containing 0-10 nM of C5a, preferably derived from the same species of mammal as are the neutrophils or other cells (e.g., human C5a for the human U937 cells). The top wells of the plate are filled with cell suspensions (compound or vehicle-treated). The plate is then placed in a tissue culture incubator for 60 minutes. The top surface of the plate is washed with PBS to remove excess cell suspension. The number of cells that have migrated into the bottom well is then determined using a fluorescence reader. Chemotaxis index (the ratio of migrated cells to total number of cells loaded) is then calculated for each compound concentration to determine an IC₅₀ value.

As a control to ensure that cells retain chemotactic ability in the presence of the compound of interest, the bottom wells of the plate may be filled with varying concentrations chemo-attractants that do not mediate chemotaxis via the C5a receptor (e.g., zymosan-activated serum (ZAS), N-formylmethionyl-leucyl-phenylalanine (FMLP) or leukotriene B4 (LTB4)), rather than C5a, under which conditions the compounds provided herein preferably do not inhibit chemotaxis.

Preferred compounds exhibit IC₅₀ values of less than 1 μM in the above assay for C5a receptor mediated chemotaxis.

### EXAMPLE 11

### Expression of a C5a Receptor

A human C5a receptor cDNA is obtained by PCR using 1) a forward primer adding a Kozak ribosome binding site and 2) a reverse primer that added no additional sequence, and 3) an aliquot of a Stratagene Human Fetal Brain cDNA library as template. The sequence of the resulting PCR product is as described by Gerard and Gerard, (1991) Nature 349:614-17. The PCR product is subdloned into the cloning vector pCR-Script AMP (STRATAGENE, La Jolla, Calif.) at the Srf I site. It is then excised using the restriction enzymes EcoRI and NotI and subcloned in the appropriate orientation for expression into the baculoviral expression vector pBacPAK 9 (CLONTECH, Palo Alto, Calif.) that has been digested with EcoRI and NotI.

### EXAMPLE 12

### Baculoviral Preparations for C5a Expression

The human C5a (hC5a) receptor baculoviral expression vector is co-transfected along with BACULOGOLD DNA (BD PharMingen, San Diego, Calif.) into Sf9 cells. The Sf9 cell culture supernatant is harvested three days post-transfection. The recombinant virus-containing supernatant is serially diluted in Hink's TNM-FH insect medium (JRH Biosciences, Lenexa, Kans.) supplemented Grace's salts and with 4.1 mM L-Gln, 3.3 g/L LAH, 3.3 g/L ultrafiltered yeastolate and 10% heat-inactivated fetal bovine serum (hereinafter “insect medium”) and plaque assayed for recombinant plaques. After four days, recombinant plaques are selected and harvested into 1 ml of insect medium for amplification. Each 1 ml volume of recombinant baculovirus (at passage 0) is used to infect a separate T25 flask containing 2×10⁶ Sf9 cells in 5 mls of insect medium. After five days of incubation at 27° C., supernatant medium is harvested from each of the T25 infections for use as passage 1 inoculum.

Two of seven recombinant baculoviral clones are then chosen for a second round of amplification, using 1 ml of passage 1 stock to infect 1×10⁸ cells in 100 ml of insect medium divided into 2 T175 flasks. Forty-eight hours post infection, passage 2 medium from each 100 ml prep is harvested and plaque assayed for titer. The cell pellets from the second round of amplification are assayed by affinity binding as described below to verify recombinant receptor expression. A third round of amplification is then initiated using a multiplicity of infection of 0.1 to infect a liter of Sf9 cells. Forty hours post-infection the supernatant medium is harvested to yield passage 3 baculoviral stock.

The remaining cell pellet is assayed for affinity binding using the “Binding Assays” essentially as described by DeMartino et al. (1994) J. Biol. Chem. 269:14446-50 at page 14447, adapted as follows. Radioligand is 0.005-0.500 nM [¹²⁵I]C5a (human recombinant; New England Nuclear Corp., Boston, Mass.); the hC5a receptor-expressing baculoviral cells are used instead of 293 cells; the assay buffer contains 50 mM Hepes pH. 7.6, 1 mM CaCl₂, 5 mM MgCl₂, 0.1% BSA, pH 7.4, 0.1 mM bacitracin, and 100 KIU/ml aprotinin; filtration is carried out using GF/C WHATMAN filters (presoaked in 1.0% polyethyeneimine for 2 hours prior to use); and the filters are washed twice with 5 mLs cold binding buffer without BSA, bacitracin, or aprotinin.

Titer of the passage 3 baculoviral stock is determined by plaque assay and a multiplicity of infection, incubation time course, binding assay experiment is carried out to determine conditions for optimal receptor expression. A multiplicity of infection of 0.1 and a 72-hour incubation were the best infection parameters found for hC5a receptor expression in up to 1-liter Sf9 cell infection cultures.

### EXAMPLE 13

### Baculoviral Infections

Log-phase Sf9 cells (INVITROGEN Corp., Carlsbad Calif.) are infected with one or more stocks of recombinant baculovirus followed by culturing in insect medium at 27° C. Infections are carried out either only with virus directing the expression of the hC5a receptor or with this virus in combination with three G-protein subunit-expression virus stocks: 1) rat Gαᵢ₂ G-protein-encoding virus stock (BIOSIGNAL #V5J008), 2) bovine b1 G-protein-encoding virus stock (BIOSIGNAL #V5H012), and 3) human g2 G-protein-encoding virus stock (BIOSIGNAL #V6B003), all of which may be obtained from BIOSIGNAL Inc. (Montreal, Canada).

The infections are conveniently carried out at a multiplicity of infection of 0.1:1.0:0.5:0.5. At 72 hours post-infection, a sample of cell suspension is analyzed for viability by trypan blue dye exclusion, and the remaining Sf9 cells are harvested via centrifugation (3000 rpm/10 minutes/4° C.).

### EXAMPLE 14

### Purified Recombinant Insect Cell Membranes

Sf9 cell pellets are resuspended in homogenization buffer (10 mM HEPES, 250 mM sucrose, 0.5 μg/mL leupeptin, 2 μg/mL Aprotinin, 200 μM PMSF, and 2.5 mM EDTA, pH 7.4) and homogenized using a POLYTRON homogenizer (setting 5 for 30 seconds). The homogenate is centrifuged (536×g/10 minutes/4° C.) to pellet the nuclei. The supernatant containing isolated membranes is decanted to a clean centrifuge tube, centrifuged (48,000×g/30 minutes, 4° C.) and the resulting pellet resuspended in 30 mL homogenization buffer. This centrifugation and resuspension step is repeated twice. The final pellet is resuspended in ice cold Dulbecco's PBS containing 5 mM EDTA and stored in frozen aliquots at −80° C. until needed. The protein concentration of the resulting membrane preparation (hereinafter “P2 membranes”) is conveniently measured using a Bradford protein assay (Bio-Rad Laboratories, Hercules, Calif.). By this measure, a 1-liter culture of cells typically yields 100-150 mg of total membrane protein.

### EXAMPLE 15

### Radioligand Binding Assays

Purified P2 membranes, prepared by the method given above, are resuspended by Dounce homogenization (tight pestle) in binding buffer (50 mM Hepes pH. 7.6, 120 mM NaCl, 1 mM CaCl₂, 5 mM MgCl₂, 0.1% BSA, pH 7.4, 0.1 mM bacitracin, 100 KIU/mL aprotinin).

For saturation binding analysis, membranes (5-50 μg) are added to polypropylene tubes containing 0.005-0.500 nM [¹²⁵I]C5a (human (recombinant), New England Nuclear Corp., Boston, Mass.) with a final assay volume of 0.25 ml. Nonspecific binding is determined in the presence of 300 nM hC5a (Sigma Chemical Co., St. Louis, Mo.) and accounted for less than 10% of total binding. For evaluation of guanine nucleotide effects on receptor affinity, GTPγS is added to duplicate tubes at the final concentration of 50 μM.

For competition analysis, membranes (5-50 μg) are added to polypropylene tubes containing 0.030 nM [¹²⁵I]C5a (human). Non-radiolabeled displacers are added to separate assays at concentrations ranging from 10⁻¹⁰ M to 10⁻⁵ M to yield a final volume of 0.250 mL. Nonspecific binding is determined in the presence of 300 nM hC5a (Sigma Chemical Co., St. Louis, Mo.) and accounted for less than 10% of total binding. Following a 2-hour incubation at room temperature, the reaction is terminated by rapid vacuum filtration. Samples are filtered over presoaked (in 1.0% polyethyleneimine for 2 hours prior to use) GF/C WHATMAN filters and rinsed 2 times with 5 mL cold binding buffer without BSA, bacitracin, or aprotinin. Remaining bound radioactivity is quantified by gamma counting. Kᵢ and Hill coefficient (“nH”) are determined by fitting the Hill equation to the measured values with the aid of SIGMAPLOT software.

### EXAMPLE 16

### Agonist-Induced GTP Binding

Agonist-stimulated GTP-gamma ³⁵S binding (“GTP binding”) activity can be used to identify agonist and antagonist compounds and to differentiate neutral antagonist compounds from those that possess inverse agonist activity. This activity can also be used to detect partial agonism mediated by antagonist compounds. A compound being analyzed in this assay is referred to herein as a “test compound.” Agonist-stimulated GTP binding activity is measured as follows: Four independent baculoviral stocks (one directing the expression of the hC5a receptor and three directing the expression of each of the three subunits of a heterotrimeric G-protein) are used to infect a culture of Sf9 cells as described above.

Agonist-stimulated GTP binding on purified membranes (prepared as described above) is assessed using hC5a (Sigma Chemical Co., St. Louis, Mo., USA) as agonist in order to ascertain that the receptor/G-protein-alpha-beta-gamma combination(s) yield a functional response as measured by GTP binding.

P2 membranes are resuspended by Dounce homogenization (tight pestle) in GTP binding assay buffer (50 mM Tris pH 7.0, 120 mM NaCl, 2 mM MgCl₂, 2 mM EGTA, 0.1% BSA, 0.1 mM bacitracin, 100KIU/mL aprotinin, 5 μM GDP) and added to reaction tubes at a concentration of 30 μg protein/reaction tube. After adding increasing doses of the agonist hC5a at concentrations ranging from 10⁻¹² M to 10⁻⁶ M, reactions are initiated by the addition of 100 pM GTPgamma³⁵S with a final assay volume of 0.25 ml. In competition experiments, non-radiolabeled test compounds (e.g., compounds of Formula I) are added to separate assays at concentrations ranging from 10⁻¹⁰ M to 10⁻⁵ M along with 10 nM hC5a to yield a final volume of 0.25 mL.

Neutral antagonists are those test compounds that reduce the C5a-stimulated GTP binding activity towards, but not below, baseline (the level of GTP bound by membranes in this assay in the absence of added C5a or other agonist and in the further absence of any test compound).

In contrast, in the absence of added C5a, certain preferred compounds reduce the GTP binding activity of the receptor-containing membranes below baseline, and are thus characterized as inverse agonists. If a test compound that displays antagonist activity does not reduce the GTP binding activity below baseline in the absence of the C5a agonist, it is characterized as a neutral antagonist.

An antagonist test compound that elevates GTP binding activity above baseline in the absence of added hC5a in this assay is characterized as having partial agonist activity. Preferred antagonist compounds provided herein do not elevate GTP binding activity under such conditions more than 10% above baseline, preferably not more than 5% above baseline, and most preferably not more than 2% above baseline.

Following a 60-minute incubation at room temperature, the reactions are terminated by vacuum filtration over GF/C filters (pre-soaked in wash buffer, 0.1% BSA) followed by washing with ice-cold wash buffer (50 mM Tris pH 7.0, 120 mM NaCl). The amount of receptor-bound (and thereby membrane-bound) GTPgamma³⁵S is determined by measuring the bound radioactivity, preferably by liquid scintillation spectrometry of the washed filters. Non-specific binding is determined using 10 mM GTPgammaS and typically represents less than 5 percent of total binding. Data is expressed as percent above basal (baseline). The results of these GTP binding experiments is analyzed using SIGMAPLOT software (SPSS Inc., Chicago, Ill.).

### EXAMPLE 17

### Calcium Mobilization Assays

A. Response to C5a

U937 cells are grown in differentiation media (1 mM dibutyrl cAMP in RPMI 1640 medium containing 10% fetal bovine serum) for 48 hours at 37° C. then reseeded onto 96-well plates suitable for use in a FLIPR™ Plate Reader (Molecular Devices Corp., Sunnyvale Calif.). Cells are grown an additional 24 hours (to 70-90% confluence) before the assay. The cells are then washed once with Krebs Ringer solution. FLUO-3 calcium sensitive dye (Molecular Probes, Inc. Eugene, Oreg.) is added to 10 μg/ml and incubated with the cells in Krebs Ringer solution at room temperature for 1 to 2 hours. The 96 well plates are then washed to remove excess dye. Fluorescence responses, measured by excitation at 480 nM and emission at 530 nM, are monitored upon the addition of human C5a to the cells to a final concentration of 0.01-30.0 nM, using the FLIPR™ device (Molecular Devices). Differentiated U937 cells typically exhibit signals of 5,000-50,000 Arbitrary Fluorescent Light Units in response to agonist stimulation.

B. Assays for Determination of ATP Responses

Differentiated U937 cells (prepared and tested as described above under “A. Response to C5a”) are stimulated by the addition of ATP (rather than C5a) to a final concentration of 0.01 to 30 μM. This stimulation typically triggers a signal of 1,000 to 12,000 arbitrary fluorescence light units. Certain preferred compounds produce less than a 10%, preferably less than a 5%, and most preferably less than a 2% alteration of this calcium mobilization signal when this control assay is carried out in the presence or absence of the compounds.

C. Assays for the Identification of Receptor Modulatory Agents: Antagonists and Agonists

Those of skill in the art will recognize that the calcium mobilization assay described above may be readily adapted for identifying test compounds as having agonist or antagonist activity at the human C5a receptor.

For example, in order to identify antagonist compounds, differentiated U937 cells are washed and incubated with Fluo-3 dye as described above. One hour prior to measuring the fluorescence signal, a subset of the cells is incubated with a 1 μM concentration of at least one compound to be tested. The fluorescence response upon the subsequent addition of 0.3 nM (final concentration) human recombinant C5a is monitored using the FLIPR™ plate reader. Antagonist compounds elicit at least a 2-fold decrease in the fluorescence response relative to that measured in the presence of human C5a alone. Preferred antagonist compounds elicit at least a 5-fold, preferably at least a 10-fold, and more preferably at least a 20-fold decrease in the fluorescence response relative to that measured in the presence of human C5a alone. Agonist compounds elicit an increase in fluorescence without the addition of C5a, which increase will be at least partially blocked by a known C5a receptor antagonist.

If multiple concentrations of antagonist compound are examined as described in the preceding paragraph, the concentration required to provide a 50% inhibition of the 0.3 nM C5a response (hereafter referred to as IC₅₀) can be determined. The IC₅₀ value is calculated by fitting the percent inhibition calculated from the relative fluorescence units (RFU) obtained at the FLIPR against the concentration of antagonist compound to the following equation:
y=m₁*(1/(1+(m₂/m₀)^{m3})),
where y=% Inhibition of C5a-induced signal, m₀=antagonist compound concentration, m₁=maximum inhibition of C5a-induced signal by highest concentration of antagonist compound, m₂=IC₅₀, and m₃=Hill slope. The data are fit to this equation using a least squares regression to determine IC₅₀ and Hill slope. The Kᵢ is calculated using the Cheng-Prusoff equation:
Ki=IC₅₀/(1+[L]/K_{d}),
where IC₅₀ is determined as described above, [L] is the C5a concentration used to test antagonist compound activity, and K_{d} is the dissociation constant of recombinant human C5a.

### EXAMPLE 18

### Assays to Evaluate Agonist Activity of Small Molecule C5a Receptor Antagonists

Certain preferred compounds of Formula I are C5a receptor antagonists that do not possess significant (e.g., greater than 5%) agonist activity in any of the C5a mediated functional assays discussed herein. Such agonist activity can be evaluated, for example, in the assay of C5a induced GTP binding given above, by measuring small molecule mediated GTP binding in the absence of the natural agonist, C5a. Similarly, in a calcium mobilization assay such as the assay described above a small molecule compound can be directly assayed for the ability of the compound to stimulate calcium levels in the absence of the natural agonist, C5a. The preferred extent of C5a agonist activity exhibited by certain compounds provided herein is less than 10%, more preferably less than 5% and most preferably less than 2% of the response elicited by the natural agonist, C5a.

### EXAMPLE 19

### MDCK Toxicity Assay

This Example illustrates the evaluation of compound toxicity using a Madin Darby canine kidney (MDCK) cell cytotoxicity assay.

1 μL of test compound is added to each well of a clear bottom 96-well plate (PACKARD, Meriden, Conn.) to give final concentration of compound in the assay of 10 micromolar, 100 micromolar or 200 micromolar. Solvent without test compound is added to control wells.

MDCK cells, ATCC no. CCL-34 (American Type Culture Collection, Manassas, Va.), are maintained in sterile conditions following the instructions in the ATCC production information sheet. Confluent MDCK cells are trypsinized, harvested, and diluted to a concentration of 0.1×10⁶ cells/ml with warm (37° C.) medium (VITACELL Minimum Essential Medium Eagle, ATCC catalog # 30-2003). 100 μL of diluted cells is added to each well, except for five standard curve control wells that contain 100 μL of warm medium without cells. The plate is then incubated at 37° C. under 95% O₂, 5% CO₂ for 2 hours with constant shaking. After incubation, 50 μL of mammalian cell lysis solution” (available as a component of the PACKARD (Meriden, Conn.) ATP-LITE-M Luminescent ATP detection kit) is added per well, the wells are covered with PACKARD TOPSEAL stickers, and plates are shaken at approximately 700 rpm on a suitable shaker for 2 minutes.

Compounds causing toxicity will decrease ATP production, relative to untreated cells. The PACKARD ATP-LITE-M Luminescent ATP detection kit, product no. 6016941, is generally used according to the manufacturer's instructions to measure ATP production in treated and untreated MDCK cells. PACKARD ATP LITE-M reagents are allowed to equilibrate to room temperature. Once equilibrated, the lyophilized substrate solution is reconstituted in 5.5 mL of substrate buffer solution (from kit). Lyophilized ATP standard solution is reconstituted in deionized water to give a 10 mM stock. For the five control wells, 10 μL of serially diluted PACKARD standard is added to each of the standard curve control wells to yield a final concentration in each subsequent well of 200 nM, 100 nM, 50 nM, 25 nM and 12.5 nM. PACKARD substrate solution (50 μL) is added to all wells, which are then covered, and the plates are shaken at approximately 700 rpm on a suitable shaker for 2 minutes. A white PACKARD sticker is attached to the bottom of each plate and samples are dark adapted by wrapping plates in foil and placing in the dark for 10 minutes. Luminescence is then measured at 22° C. using a luminescence counter (e.g., PACKARD TOPCOUNT Microplate Scintillation and Luminescence Counter or TECAN SPECTRAFLUOR PLUS), and ATP levels calculated from the standard curve. ATP levels in cells treated with test compound(s) are compared to the levels determined for untreated cells. Cells treated with 10 μM of a preferred test compound exhibit ATP levels that are at least 80%, preferably at least 90%, of the untreated cells. When a 100 μM concentration of the test compound is used, cells treated with preferred test compounds exhibit ATP levels that are at least 50%, preferably at least 80%, of the ATP levels detected in untreated cells.

1. A compound of Formula IA: 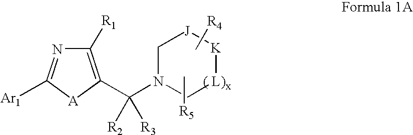
or a pharmaceutically acceptable salt thereof, wherein
A is oxygen, sulfur or NR;
R is C₁-C₇alkyl, C₂-C₇alkenyl, C₂-C₇alkynyl, C₁-C₆haloalkyl, (C₃-C₁₀carbocycle)C₁-C₄alkyl or (4- to 7-membered heterocycloalkyl)C₁-C₄alkyl, each of which is substituted with from 0 to 3 substituents independently chosen from hydroxy, halogen, amino, cyano, oxo, C₁-C₄alkyl, C₁-C₄alkoxy and C₁-C₂alkoxycarbonyl;
x is 0, 1 or 2;
J, K and each occurrence of L are chosen from oxygen, sulfur, NH and CH₂; such that no more than one of J, K and L is chosen from oxygen, sulfur and NH;
R₁ is chosen from:
i) hydrogen, hydroxy, halogen, amino, cyano, nitro, —CHO, —CONH₂, C₁-C₆haloalkyl and C₁-C₆haloalkoxy;
ii) C₁-C₆alkyl, C₂-C₇alkenyl, C₂-C₇alkynyl, C₂-C₆alkanoyl, C₁-C₆alkoxy, (C₃-C₇cycloalkyl)C₀-C₄alkyl, (4- to 10-membered heterocycloalkyl)C₀-C₄alkyl, mono- and di-(C₁-C₆alkyl)aminoC₀-C₆alkyl, mono- and di-(C₁-C₆alkyl)carboxamide, C₁-C₆alkoxycarbonyl, —SOₙ(C₁-C₆alkyl), —NHSOₙC₁-C₆alkyl, —(C₀-C₆alkyl)SOₙ(C₁-C₆alkyl), —SOₙN(C₁-C₆alkyl)(C₁-C₆alkyl), and —SOₙ-phenyl, wherein each n is independently 0, 1 or 2, and each of which is substituted with from 0 to 3 substituents independently chosen from hydroxy, halogen, amino, cyano, oxo, C₁-C₄alkyl, C₁-C₄alkoxy and C₁-C₂alkoxycarbonyl; and
iii) naphthyl, phenyl and 5- to 10-membered heteroaryl, each of which is substituted with from 0 to 3 substituents independently chosen from R₁₁;
R₂ and R₃ are independently hydrogen or C₁-C₆alkyl;
R₄ represents 1 substituent chosen from:
i) C₁-C₆alkyl, C₂-C₆alkenyl, C₂-C₆alkynyl, C₁-C₆alkoxycarbonyl, (C₃-C₇cycloalkyl)C₀-C₄alkyl and hexahydro-1,3-benzodioxolyl;
ii) aryl having 1 ring or 2 fused or pendant rings;
iii) (4- to 10-membered heterocycloalkyl)C₀-C₄alkyl;
iv) phenyl fused to a 5- to 7-membered saturated or partially unsaturated ring that (a) has 0, 1 or 2 ring atoms independently chosen from N, O and S, with remaining ring atoms being carbon, and (b) is substituted with from 0 to 3 substituents independently chosen from halogen, C₁-C₈alkyl, C₁-C₈alkoxy, C₁-C₈haloalkyl, C₁-C₈haloalkoxy;
v) (5- to 10-membered heteroaryl)C₀-C₄alkyl, having 1 ring or 2 fused or pendant rings, from 5 to 7 members in each ring, and in at least one ring from 1 to 3 heteroatoms independently selected from N, O, and S, wherein R₄ is not pyrimidyl; and
vi) groups that are taken together with an R₅ moiety to form a fused phenyl or pyridyl ring;
wherein each of i), ii), iii), iv), v) and vi) is substituted with from 0 to 3 substituents independently chosen from R₁₁;
R₅ represents from 0 to 3 substituents independently chosen from hydroxy, halogen, amino, cyano, nitro, —CHO, —CONH₂, C₁-C₂haloalkyl, C₁-C₂haloalkoxy, C₁-C₆alkyl, C₁-C₆alkoxy, C₃-C₇cycloalkylC₀-C₄alkyl, mono- and di-(C₁-C₆alkyl)aminoC₀-C₆alkyl, optionally substituted phenyl, and groups that are taken together with R₄ to form a fused, optionally substituted phenyl or pyridyl ring; and
Ar₁ represents
i) phenyl or naphthyl, each of which is substituted with from 0 to 3 substituents independently chosen from amino, cyano, C₁-C₄alkyl, C₁-C₄alkoxy, C₁-C₂haloalkyl, C₁-C₂haloalkoxy, —COOH, —CONH₂, mono- and di-(C₁-C₄alkyl)amino, C₂-C₄alkanoyl, C₁-C₄sulfonate, C₁-C₄alkylsulfonyl, C₁-C₄alkylsulfinyl, C₁-C₄alkylthio, C₃-C₆alkanone, C₂-C₄alkyl ether, C₂-C₄alkanoyloxy, C₁-C₄alkoxycarbonyl and C₁-C₆alkylcarboxamide;
ii) phenyl fused to a 5- to 7-membered saturated or partially unsaturated ring that (a) has 0, 1 or 2 ring atoms independently chosen from N, O and S, with remaining ring atoms being carbon, and (b) is substituted with from 0 to 3 substituents independently chosen from halogen, C₁-C₄alkyl, C₁-C₄alkoxy, C₁-C₂haloalkyl and C₁-C₂haloalkoxy; or
iii) heteroaryl, having 1 ring or 2 fused or pendant rings, from 5 to 7 members in each ring, and in at least one ring from 1 to 3 heteroatoms independently selected from N, O, and S;
wherein each of ii) and iii) is substituted with from 0 to 3 substituents independently chosen from R₁₁; and
R₁₁ is independently chosen at each occurrence from hydroxy, halogen, amino, cyano, C₁-C₆alkyl, C₁-C₆alkoxy, C₁-C₆haloalkyl, C₁-C₆haloalkoxy, —COOH, —CONH₂, —SO₂NH₂, mono- and di-(C₁-C₆alkyl)amino, C₂-C₆alkanoyl, C₁-C₆sulfonate, C₁-C₆alkylsulfonyl, C₁-C₆alkylsulfinyl, C₁-C₆alkylthio, C₃-C₆alkanone, C₂-C₆alkyl ether, C₂-C₆alkanoyloxy, C₁-C₆alkoxycarbonyl and C₁-C₆alkylcarboxamide.

2. A compound or salt according to claim 1, wherein:
R is chosen from C₁-C₇alkyl, C₂-C₇alkenyl, C₂-C₇alkynyl, (C₃-C₇cycloalkyl)C₁-C₄alkyl and (4- to 7-membered heterocycloalkyl)C₁-C₄alkyl, each of which is substituted with from 0 to 3 substituents independently chosen from hydroxy, halogen, amino, cyano, oxo, C₁-C₄alkyl, C₁-C₄alkoxy and C₁-C₂alkoxycarbonyl;
R₁ is chosen from:
i) hydrogen, hydroxy, halogen, amino, cyano, nitro, —CHO, —CONH₂, C₁-C₆haloalkyl and C₁-C₆haloalkoxy;
ii) C₁-C₆alkyl, C₁-C₆alkenyl, C₁-C₆alkynyl, C₁-C₆alkoxy, (C₃-C₇cycloalkyl)C₀-C₂alkyl, (4- to 10-membered heterocycloalkyl)C₀-C₂alkyl, and mono- and di-(C₁-C₆alkyl)carboxamide, each of which is substituted with from 0 to 3 substituents independently chosen from hydroxy, halogen, amino, cyano, oxo, C₁-C₄alkyl and C₁-C₄alkoxy, and
iii) naphthyl, phenyl, pyridyl, thiazolyl, pyrimidinyl and thienyl, each of which is substituted with from 0 to 3 substituents independently chosen from hydroxy, halogen, amino, cyano, —COOH, —CONH₂, —SO₂NH₂, C₁-C₆haloalkyl, C₁-C₆haloalkoxy, C₁-C₆alkyl, C₁-C₆alkoxy, C₂-C₆alkanoyl, C₁-C₆alkylsulfonyl, C₁-C₆alkylsulfinyl, C₁-C₆alkylthio, C₃-C₆alkanone, C₂-C₆alkylether, C₂-C₆alkanoyloxy, C₁-C₆alkoxycarbonyl and C₁-C₆alkylcarboxamide;
R₄:
i) represents C₁-C₆alkyl, C₂-C₇alkenyl, C₂-C₇alkynyl, C₁-C₆alkoxycarbonyl, (C₃-C₇cycloalkyl)C₀-C₄alkyl, hexahydro-1,3-benzodioxolyl, phenyl, naphthyl or (4- to 7-membered heterocycloalkyl)C₀-C₄alkyl, each of which is substituted with from 0 to 3 substituents independently chosen from hydroxy, halogen, amino, cyano, C₁-C₄alkyl, C₁-C₄alkoxy, C₁-C₂haloalkyl, C₁-C₂haloalkoxy, —COOH, —CONH₂, mono- and di-(C₁-C₄alkyl)amino, C₂-C₄alkanoyl, C₁-C₄sulfonate, C₁-C₄alkylsulfonyl, C₁-C₄alkylsulfinyl, C₁-C₄alkylthio, C₃-C₆alkanone, C₂-C₄alkyl ether, C₂-C₄alkanoyloxy, C₁-C₄alkoxycarbonyl, and C₁-C₆alkylcarboxamide; or
ii) is phenyl fused to a 5- to 7-membered saturated or partially unsaturated ring that (a) has 0, 1 or 2 ring atoms independently chosen from N, O and S, with remaining ring atoms being carbon, and (b) is substituted with from 0 to 3 substituents independently chosen from halogen, C₁-C₄alkyl, C₁-C₄alkoxy, C₁-C₂haloalkyl and C₁-C₂haloalkoxy; or
iii) is taken together with an R₅ moiety to form a fused phenyl or pyridyl ring that is substituted with from 0 to 3 substituents independently chosen from hydroxy, halogen, amino, cyano, C₁-C₄alkyl, C₁-C₄alkoxy, C₁-C₂haloalkyl, C₁-C₂haloalkoxy, —COOH, —CONH₂, and mono- and di-(C₁-C₄alkyl)amino;
R₅ represents from 0 to 3 substituents independently chosen from hydroxy, halogen, amino, cyano, C₁-C₄alkyl, C₁-C₄alkoxy, C₁-C₂haloalkyl, C₁-C₂haloalkoxy, —COOH, —CONH₂, mono- and di-(C₁-C₄alkyl)amino, and groups that are taken together with R₄ to form a fused, optionally substituted phenyl or pyridyl ring; and
Ar₁ represents phenyl, naphthyl, pyridyl, pyrimidinyl, pyridizinyl, pyrazinyl, pyrazolyl, imidazolyl, thiazolyl, isothiazolyl, pyrrolyl, oxazolyl, furanyl, indazolyl or thienyl, each of which is substituted with from 0 to 3 substituents independently chosen from amino, cyano, C₁-C₄alkyl, C₁-C₄alkoxy, C₁-C₂haloalkyl, C₁-C₂haloalkoxy, —COOH, —CONH₂, mono- and di-(C₁-C₄alkyl)amino, C₂-C₄alkanoyl, C₁-C₄sulfonate, C₁-C₄alkylsulfonyl, C₁-C₄alkylsulfinyl, C₁-C₄alkylthio, C₃-C₆alkanone, C₂-C₄alkyl ether, C₂-C₄alkanoyloxy, C₁-C₄alkoxycarbonyl and C₁-C₆alkylcarboxamide.

3. A compound or salt according to claim 1, wherein A is oxygen.

4. A compound or salt according to claim 1, wherein A is sulfur.

5. A compound or salt according to claim 1, wherein A is NR.

6. A compound or salt according to claim 1, wherein the compound satisfies Formula II:

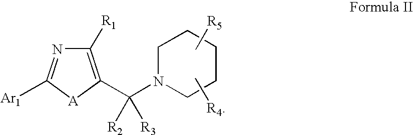

7. A compound or salt according to claim 1, wherein the compound satisfies Formula III:

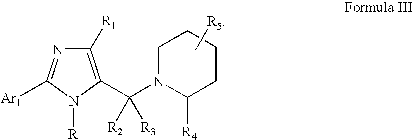

8. A compound or salt according to claim 1, wherein the compound satisfies Formula IV:

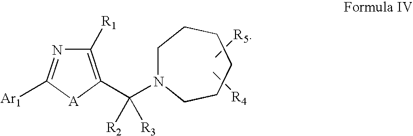

9. A compound or salt according to claim 1, wherein the compound satisfies Formula V:

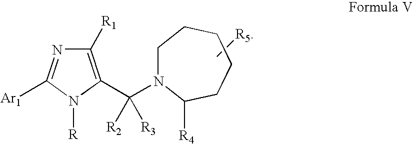

10. A compound or salt according to claim 1, wherein the compound satisfies Formula VI:

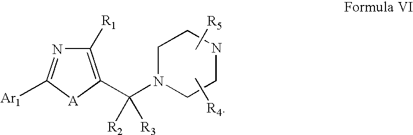

11. A compound or salt according to claim 1, wherein the compound satisfies Formula VIII:

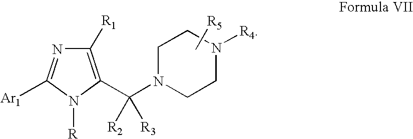

12. A compound or salt according to claim 1, wherein the compound satisfies Formula VIII: 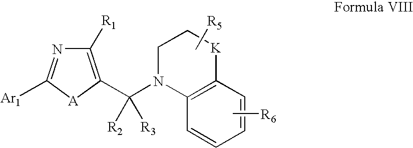
wherein:
K is CH₂ or NH; and
R₆ represents from 0 to 3 substituents independently chosen from hydroxy, halogen, amino, cyano, C₁C₄alkyl, C₁-C₄alkoxy, C₁-C₂haloalkyl, C₁-C₂haloalkoxy, —COOH, —CONH₂ and mono- and di-(C₁-C₄alkyl)amino.

13. (canceled)

14. A compound or salt according to claim 1, wherein the compound satisfies Formula IX:

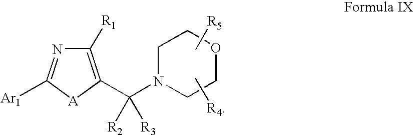

15. A compound or salt according to claim 1, wherein the compound satisfies Formula X:

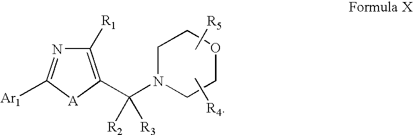

16. A compound or salt according to claim 1, wherein the compound satisfies Formula XI:

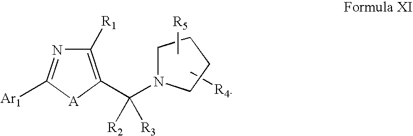

17. A compound or salt according to claim 1, wherein the compound satisfies Formula XII: 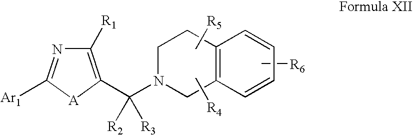
wherein R₆ represents from 0 to 3 substituents independently chosen from hydroxy, halogen, amino, cyano, C₁C₄alkyl, C₁-C₄alkoxy, C₁-C₂haloalkyl, C₁-C₂haloalkoxy, —COOH, —CONH₂ and mono- and di-(C₁-C₄alkyl)amino.

18. A compound or salt according to claim 1, wherein R₂ and R₃ are both hydrogen.

19. A compound or salt according to claim 1, wherein Ar₁ is phenyl, pyridyl, indazolyl or thienyl, each of which is substituted with 0 to 3 substituents independently chosen from C₁-C₄alkyl, C₁-C₄alkoxy, C₁-C₂haloalkyl, C₁-C₂haloalkoxy and mono- and di-(C₁-C₂alkyl)amino.

20-22. (canceled)

23. A compound or salt according to claim 2, wherein R, is:
i) halogen;
ii) C₁-C₆alkyl, C₂-C₆alkenyl, C₁-C₆alkoxy, (C₃-C₇cycloalkyl)C₀-C₄alkyl, pyrrolidinylC₀-C₂alkyl, morpholinylC₀-C₂alkyl, piperinylC₀-C₂alkyl or piperazinylC₀-C₂alkyl, each of which is substituted with from 0 to 3 substituents independently chosen from hydroxy, halogen, amino, cyano, oxo, C₁-C₄alkyl and C₁-C₄alkoxy; or
iii) phenyl or pyridyl, each of which is substituted with from 0 to 3 substituents independently chosen from hydroxy, halogen, amino, cyano, —COOH, —CONH₂, —SO₂NH₂, C₁-C₂haloalkyl, C₁-C₂haloalkoxy, C₁-C₆alkyl, C₁-C₆alkoxy, and mono- and di-(C₁-C₄alkyl)amino.

24-25. (canceled)

26. A compound or salt according to claim 5, wherein R is C₁-C₇alkyl, C₂-C₇alkenyl, (C₃-C₇cycloalkyl)C₁-C₄alkyl or (1,3-dioxylan-2-yl)C₁-C₄alkyl, each of which is substituted with from 0 to 3 substituents independently chosen from hydroxy, halogen, amino, cyano, oxo, C₁-C₄alkyl and C₁-C₄alkoxy.

27. (canceled)

28. A compound or salt according to claim 3, wherein R₄ is C₁-C₆alkyl, C₁-C₆alkoxycarbonyl or C₃-C₇ cycloalkyl, each of which is substituted with from 0 to 3 substituents independently chosen from hydroxy, halogen, amino, cyano, C₁-C₂alkyl, C₁-C₂alkoxy, C₁-C₂haloalkyl, C₁-C₂haloalkoxy, —COOH, —CONH₂, and mono- and di-(C₁-C₄alkyl)amino.

29. A compound or salt according to claim 3, wherein R₄ is phenylC₀-C₁alkyl, pyridylC₀-C₁alkyl, pyrimidylC₀-C₁alkyl, thienylC₀-C₁alkyl, naphthylC₀-C₁alkyl, indolylC₀-C₁alkyl, benzoxadiazolylC₀-C₁alkyl, benzoxazolylC₀-C₁alkyl, quinazolinylC₀-C₁alkyl, benzothiazolylC₀-C₁alkyl or benzimidazolylC₀-C₁alkyl, each of which is substituted with from 0 to 2 substituents independently chosen from hydroxy, halogen, amino, cyano, C₁-C₂ alkyl, C₁-C₂alkoxy, C₁-C₂haloalkyl, C₁-C₂haloalkoxy and mono- and di-(C₁-C₂alkyl)amino.

30-35. (canceled)

36. A compound or salt according to claim 3, wherein R₅ represents from 0 to 3 substituents independently chosen from hydroxy, halogen, C₁-C₂alkyl, and C₁-C₂alkoxy.

37-39. (canceled)

40. A pharmaceutical composition comprising at least one compound or salt according to claim 1, in combination with a physiologically acceptable carrier or excipient.

41. A pharmaceutical composition according claim 40, wherein the pharmaceutical composition is formulated as an injectible fluid, an aerosol, a cream, a gel, a pill, a capsule, a syrup, or a transdermal patch.

42. A method for inhibiting signal-transducing activity of a cellular C5a receptor, comprising contacting a cell expressing C5a receptor with at least one compound or salt according to claim 1, and thereby reducing signal transduction by the C5a receptor.

43. A method according to claim 42, wherein the cell is contacted in vivo in an animal.

44. A method according to claim 43, wherein the animal is a human.

45. (canceled)

46. A method for inhibiting binding of C5a to C5a receptor in a human patient, comprising contacting cells expressing C5a receptor with at least one compound or salt according to claim 1, in an amount sufficient to detectably inhibit C5a binding to cells expressing a cloned C5a receptor in vitro, and thereby inhibiting binding of C5a to the C5a receptor in the patient.

47. A method for treating a patient suffering from rheumatoid arthritis, psoriasis, cardiovascular disease, reperfusion injury, or bronchial asthma comprising administering to the patient a C5a receptor modulatory amount of a compound or salt according to claim 1.

48. A method for treating a patient suffering from stroke, myocardial infarction, atherosclerosis, ischemic heart disease, or ischemia-reperfusion injury comprising administering to the patient a C5a receptor modulatory amount of a compound or salt according to claim 1.

49. A method for treating a patient suffering from cystic fibrosis or sepsis, comprising administering to a patient in need of such treatment a C5a receptor modulatory amount of a compound or salt according to claim 1.

50. A method for inhibiting C5a receptor-mediated cellular chemotaxis, comprising contacting mammalian white blood cells with a C5a receptor modulatory amount of a compound or salt according to claim 1.

51-55. (canceled)

56. A compound or salt according to claim 4, wherein R₄ is C₁-C₆alkyl, C₁-C₆alkoxycarbonyl or C₃-C₇ cycloalkyl, each of which is substituted with from 0 to 3 substituents independently chosen from hydroxy, halogen, amino, cyano, C₁-C₂alkyl, C₁-C₂alkoxy, C₁-C₂haloalkyl, C₁-C₂haloalkoxy, —COOH, —CONH₂, and mono- and di-(C₁-C₄alkyl)amino.

57. A compound or salt according to claim 5, wherein R₄ is C₁-C₆alkyl, C₁-C₆alkoxycarbonyl or C₃-C₇ cycloalkyl, each of which is substituted with from 0 to 3 substituents independently chosen from hydroxy, halogen, amino, cyano, C₁-C₂alkyl, C₁-C₂alkoxy, C₁-C₂haloalkyl, C₁-C₂haloalkoxy, —COOH, —CONH₂, and mono- and di-(C₁-C₄alkyl)amino.

58. A compound or salt according to claim 4, wherein R₄ is phenylC₀-C₁alkyl, pyridylC₀-C₁alkyl, pyrimidylC₀-C₁alkyl, thienylC₀-C₁alkyl, naphthylC₀-C₁alkyl, indolylC₀-C₁alkyl, benzoxadiazolylC₀-C₁alkyl, benzoxazolylC₀-C₁alkyl, quinazolinylC₀-C₁alkyl, benzothiazolylC₀-C₁alkyl or benzimidazolylC₀-C₁alkyl, each of which is substituted with from 0 to 2 substituents independently chosen from hydroxy, halogen, amino, cyano, C₁-C₂ alkyl, C₁-C₂alkoxy, C₁-C₂haloalkyl, C₁-C₂haloalkoxy and mono- and di-(C₁-C₂alkyl)amino.

59. A compound or salt according to claim 5, wherein R₄ is phenylC₀-C₁alkyl, pyridylC₀-C₁alkyl, pyrimidylC₀-C₁alkyl, thienylC₀-C₁alkyl, naphthylC₀-C₁alkyl, indolylC₀-C₁alkyl, benzoxadiazolylC₀-C₁alkyl, benzoxazolylC₀-C₁alkyl, quinazolinylc₀-C₁alkyl, benzothiazolylC₀-C₁alkyl or benzimidazolylC₀-C₁alkyl, each of which is substituted with from 0 to 2 substituents independently chosen from hydroxy, halogen, amino, cyano, C₁-C₂ alkyl, C₁-C₂alkoxy, C₁-C₂haloalkyl, C₁-C₂haloalkoxy and mono- and di-(C₁-C₂alkyl)amino.

60. A compound or salt according to claim 4, wherein R₅ represents from 0 to 3 substituents independently chosen from hydroxy, halogen, C₁-C₂alkyl, and C₁-C₂alkoxy.

61. A compound or salt according to claim 5, wherein R₅ represents from 0 to 3 substituents independently chosen from hydroxy, halogen, C₁-C₂alkyl, and C₁-C₂alkoxy.
